(19) United States
(12) Patent Application Publication
(10) Pub. No.: US 2025/0277051 A1
(43) Pub. Date: Sep. 4, 2025

(54) ANTI-CD38 ANTIBODIES, ANTI-CD3 ANTIBODIES, AND BISPECIFIC ANTIBODIES, AND USES THEREOF

(71) Applicant: HANGZHOU UNOGEN BIOTECH, LTD, Hangzhou (CN)

(72) Inventors: Mark CHIU, Lower Gwynedd, PA (US); Man-Cheong FUNG, Lower Gwynedd, PA (US); Mark TORNETTA, Lower Gwynedd, PA (US); Brian WHITAKER, Lower Gwynedd, PA (US); Pu PU, Hong Kong (CN); Ying JIN, Hong Kong (CN); Chen PENG, Hong Kong (CN); Kenneth Cheung KWONG, Yardley, PA (US); Ao YU, Shanghai (CN); Glenn Mark ANDERSON, Eagleville, PA (US)

(21) Appl. No.: 18/294,406

(22) PCT Filed: Aug. 1, 2022
(86) PCT No.: PCT/US22/74409

§ 371 Date: Feb. 1, 2024

Related U.S. Application Data
(60) Provisional application No. 63/228,195, filed on Aug. 2, 2021.

(51) Int. Cl. C07K 16/28; A61K 39/00; A61K 47/68; A61P 35/00

(52) U.S. Cl. CPC C07K 16/2896; A61K 47/6849; A61P 35/00; C07K 16/2809; A61K 2039/505; C07K 2317/31; C07K 2317/35; C07K 2317/565; C07K 2317/569; C07K 2319/50

(57) ABSTRACT

The present disclosure relates to anti-CD3 and anti-CD38 antibodies or antigen-binding fragments thereof. The present disclosure also relates to bispecific antibodies targeting both CD3 and CD8. To expand the therapeutic index, the bispecific antibodies may contain masking domains to minimize systemic toxicity. The unmasking of the shielded bispecific antibodies occurs predominantly by proteases and enzymes in the tumor microenvironment or in the disease tissues. The present disclosure also provides a unique design that employs a human VHO single domain molecule linked to the hinge region of an antibody, which may allow better tissue penetration than conventional antibodies.

### CROSS-REFERENCE TO RELATED APPLICATION

This application claims priority to U.S. Provisional Application No. 63/228,195, filed Aug. 2, 2021, the entire contents of which are hereby incorporated by reference.

### SEQUENCE LISTING

This application contains a Sequence Listing, which has been submitted electronically in ST.26 format and is hereby incorporated by reference in its entirety.

### FIELD OF THE DISCLOSURE

The present disclosure relates to biomedicine, particularly, antibodies and bispecific antibodies directed against CD3 and CD38, nucleic acids encoding such antibodies, methods for preparing such antibodies, and methods for the treatment of cancers.

### BACKGROUND OF THE DISCLOSURE

Antibody-based therapeutics have been successful in treating a variety of diseases, including many cancers. Yet improvements to this class of drugs are still needed, particularly with respect to enhancing their clinical efficacy and safety. One avenue being explored is the engineering of additional and novel antigen binding sites into antibody-based drugs such that a single immunoglobulin molecule co-engages two different antigens.

CD38, also known as cyclic ADP ribose hydrolase, is a type II transmembrane glycoprotein with a long C-terminal extracellular domain and a short N-terminal cytoplasmic domain (Guedes, Dileepan et al. 2020, Kar, Mehrotra et al. 2020). CD38 is a glycoprotein found on the surface of many immune cells that include CD4+, CD8+, B lymphocytes, and natural killer cells (Orciani, Trubiani et al. 2008, van de Donk and Usmani 2018). While not simply being a marker of cell types, CD38 is an activator of B cells and T cells. CD38 has multiple functions, including ectoenzymatic activity and receptor-mediated regulation of cell adhesion and signal transduction as shown in FIG. 1 (Malavasi, Funaro et al. 1994, Mehta, Shahid et al. 1996, Deaglio, Mehta et al. 2001). CD38 is also a signaling enzyme responsible for the metabolism of two novel calcium messenger molecules: the enzymatic activity of CD38 mediates the synthesis of the calcium-releasing second messengers cyclic ADP-ribose (cADPR) and nicotinic acid adenine dinucleotide phosphate (NAADP) (Quarona, Zaccarello et al. 2013). Among hematopoietic cells, CD38 can mediate signaling, including lymphocyte proliferation, cytokine release, regulation of B and myeloid cell development and survival, and induction of dendritic cell maturation.

As a receptor, CD38 can attach to CD31 on the surface of T cells, thereby activating those cells to produce a variety of cytokines (Nooka, Kaufman et al. 2019). CD31, also known as PECAM1 (Platelet Endothelial Cell Adhesion Molecule-1), is a 130 kDa member of an immunoglobulin superfamily that is expressed on the surface of circulating platelets, neutrophils, monocytes, and naive B-lymphocytes. Functionally, CD31 is thought to function as an adhesion molecule. For example, endothelial cell CD38 binds to CD31 on natural killer cells for those cells to attach to the endothelium (Glaria and Valledor 2020, Zambello, Barila et al. 2020). CD38 on leukocytes can attach to CD31 on endothelial cells to allow leukocyte binding to blood vessel walls and the passage of leukocytes through blood vessel walls (Quarona, Zaccarello et al. 2013). Such an interaction of CD38 with CD31 may act in promoting survival of leukemia cells.

CD38, as a cell surface antigen, is highly expressed in several hematologic malignancies including multiple myeloma (MM) and has been proven to be a good target for immunotherapy of certain diseases as shown in FIG. 2 (Quarona, Zaccarello et al. 2013). CD38 is upregulated in many cell lines derived from various hematopoietic malignancies including non-Hodgkin's lymphoma (NHL), Burkitt's lymphoma (BL), multiple myeloma (MM), B chronic lymphocytic leukemia (B-CLL), B and T acute lymphocytic leukemia (ALL), T cell lymphoma (TCL), acute myeloid leukemia (AML), hairy cell leukemia (HCL), Hodgkin's Lymphoma (HL), and chronic myeloid leukemia (CML). Also, most primitive pluripotent stem cells of the hematopoietic system are CD38+.

Increased expression of CD38 is a diagnostic marker in chronic lymphocytic leukemia (CLL) and is associated with increased disease progression (Burgler 2015, Burgler, Gimeno et al. 2015). CD38 is a prognostic factor of acute myeloid leukemia (Zeijlemaker, Grob et al. 2019), chronic lymphocytic leukemia (Damle, Wasil et al. 1999, Malavasi, Deaglio et al. 2011), prostate cancer (Liu, Grogan et al. 2016, Stone 2017), pancreatic cancer (Zhang, Yang et al. 2019), acute B lymphoblastic leukemia (Jiang, Wu et al. 2016), lung cancer (Xu, Chen et al. 2015), hepatocellular cancer (Lam, Ng et al. 2019), and triple-negative breast cancer (Yeong, Lim et al. 2018). CD38 is also expressed heterogeneously in colorectal cancer (CRC) (Perenkov, Novikov et al. 2012) but does not depend on tumor localization, tumor grade, or presence of metastases. CD38 is also a putative functional marker for side population cells in human nasopharyngeal carcinoma cell lines (Zheng, Liao et al. 2016) and may serve a carcinogenic role in nasopharyngeal carcinoma cells by affecting energy metabolism (Ge, Long et al. 2019).

Since CD38 expression is high and uniform on malignant lymphatic cancers and is low on normal lymphoid and myeloid cells and on non-hematopoietic tissues, several biologics therapies were developed (Morandi, Airoldi et al. 2019). In chronic lymphocytic leukemia, CD38 expression is high, and preclinical studies on the use of daratumumab in monotherapy or combination therapy have demonstrated considerable efficacy. CD38 targeting is being used to treat multiple myeloma (MM) and chronic lymphocytic leukemia (CLL). Monoclonal antibodies targeting CD38, such as daratumumab, have shown good therapeutic efficacy in MM, both alone (Usmani, Weiss et al. 2016) and in combination with other standard-of-care regimens (Dimopoulos, Oriol et al. 2016, Palumbo, Chanan-Khan et al. 2016). Such therapeutic approaches have further prolonged the 5-10-year survival rates of MM patients.

However, many patients eventually relapse because of resistance mechanisms, including fragment crystallizable gamma receptor (FcγR)-dependent downregulation of CD38 on tumor cells, as well as inhibition of complement-dependent cytotoxicity, antibody-dependent cell mediated cytotoxicity, and antibody-dependent cellular phagocytosis as shown in FIG. 4 (van de Donk and Usmani 2018). Thus, novel therapeutic approaches are urgently needed.

Likewise, in other lymphoproliferative disorders, preclinical and clinical data have not been as compelling. In these cases, CD38 overexpression can contribute to resistance to checkpoint inhibitors, prompting numerous clinical trials in Hodgkin and non-Hodgkin lymphoma to investigate whether blocking CD38 enhances the efficacy of checkpoint inhibitors. In the hypoxic tumor microenvironment, NAD+ is released by the salvage pathway and hydrolyzed by CD38 to form ADP ribose. This is further degraded to AMP through the CD38-CD203a-CD73 pathway. Following this, CD73 dephosphorylates AMP to adenosine. Accumulated extracellular adenosine then binds to receptors on a range of immune cells, impeding their infiltration and activation. This pathway forms an alternative immunosuppressive mechanism to the PD-1/PD-L1 pathway, and inhibition of the adenosine pathway has been shown to reduce immunosuppression in the tumor microenvironment (Ma, Deng et al. 2017, Boison and Yegutkin 2019).

The CD38-NAD signaling pathway seems to have a relevant role in the formation of a suppressive tumor microenvironment and promotes the activity of inhibitory cell types, such as MDSCs, Tregs, Bregs, and certain subtypes of NK cells. Additionally, it is an important driver of resistance to PD-1/PD-L1 checkpoint inhibitors. CD38 cytotoxic antibodies can, therefore, exhibit direct on-tumor activity as well as indirect immunomodulatory anti-tumoral effects. They have been used to treat CD38 positive tumors, specifically MM, with considerable efficacy and a manageable toxicity profile. Substantial preclinical evidence supports their use also in CLL, both as monotherapy and combination therapy with certain agents, such as BTK inhibitors. On the contrary, other lymphoid malignancies appear to be less sensitive to anti-CD38 antibodies, with evidence of modest activity of single agent daratumumab in various types of NHL. Daratumumab and newly developed anti-CD38 antibodies, such as isatuximab, can find application in combination with standard regimens to enhance cytotoxic response or with checkpoint inhibitors to overcome acquired resistance. Furthermore, anti-CD38 antibodies have yet to be investigated in T-cell lymphomas (Calabretta and Carlo-Stella 2020). Thus, there is a need to have a stronger and selective CD38 therapeutic agent that can be used for such indications with better efficacy and safety profile.

In prostate cancer, CD38 inhibits tumor metabolism and proliferation by reducing cellular NAD+ pools (Chmielewski, Bowlby et al. 2018). CD38 is highly expressed in cervical cancer cells and enhances the proliferation and inhibits the apoptosis of cervical cancer cells by affecting mitochondrial functions (Liao, Xiao et al. 2014). High expression of CD38 predicted prognosis in esophageal squamous cell carcinoma patients (Liao, Xiao et al. 2017). CD38 expression or absence showed prognostic value for lung cancer (Karimi-Busheri, Zadorozhny et al. 2011). CD38 knockout suppresses tumorigenesis in mice and clonogenic growth of human lung cancer cells (Bu, Kato et al. 2018). Thus, it was concluded that anti-CD38 treatment may have therapeutic potential in lung cancer (Bu, Kato et al. 2018).

Besides being a marker of cell activation in leukemias, myelomas, and solid tumors, CD38 has been connected to HIV infection, Type II diabetes mellitus, bone metabolism, as well as some genetically determined conditions (Marlein, Piddock et al. 2019). CD38 overexpression has been shown to facilitate CD4 T cell depletion in HIV infection in animal models (Rodriguez-Alba et al 2019). Increased catalytic activity of CD38 may decrease CD4 T cells' cytoplasmic nicotinamide adenine dinucleotide (NAD), giving rise to the chronic “Warburg effect.” This would then decrease mitochondrial function. Meanwhile, ADPR and cADPR, which are the key catalytic products of CD38, may be carried to the cytoplasm where they can activate calcium channels and increase cytoplasmic Ca²⁺ concentrations, further altering mitochondrial integrity. These mechanisms would decrease the viability and regenerative capacity of CD4 T cells. Thus, the shutdown of CD38 activity can improve CD4 T cell activity.

Overall, as an alternative, a combination of two antibodies (Abs) that recognize distinct, non-overlapping epitopes of CD38 can mediate potent complement dependent cytotoxicity (CDC), in contrast to the Ab monotherapy with only weak CDC capacity (Schutze, Petry et al. 2018). Similarly, combining daratumumab with one of these Abs that recognizes a non-overlapping epitope resulted in dramatically enhanced CDC. Further, introducing the E345R HexaBody mutation into the CH3 domain strongly enhanced the CDC potency of these Abs to CD38-expressing cells.

To achieve a stronger patient response, T cell redirected killing is a desirable mode of action in many therapeutic areas. Various bispecific antibody formats have been shown to mediate T cell redirection both in pre-clinical and clinical investigations (May, Sapra et al. 2012, Frankel and Baeuerle 2013). These include tandem of scFv fragments and diabody based formats with only a few examples of Fc based bispecific antibody formats reported (Moore, Bautista et al. 2011 May, Sapra et al. 2012, Frankel and Baeuerle 2013). Bispecific formats that encompass a human Fc region have longer circulation half-lives, which may result in enhanced efficacy and/or less frequent dosing regimens. Among possible Fc-based bispecific formats, one preferred format to redirect T cell killing is the so-called heavy chain hetero-dimer format. This format is of particular interest as it does not allow aggregation of multiple copies of human CD3 molecules at the T cell surface thereby preventing any T cell inactivation (Klein, Sustmann et al. 2012).

Bispecific T-cell engaging (BiTE) antibodies belong to a new class of immunotherapeutic agents that can recognize, on the one hand, a specific antigen on the surface of the target cells (i.e., tumor antigen) and, on the other hand, the CD38 chain on T lymphocytes (Fumey, Koenigsdorf et al. 2017). By activating T cells via the CD3 complex and recruiting them in proximity to the target cells, BiTE antibodies efficiently induce T-cell-mediated cytotoxicity (Brischwein, Schlereth et al. 2006). In MM, bispecific antibodies recognizing B-cell maturation antigen or FcRH5 (CD307) have been shown to eliminate tumor plasma cells in preclinical models (Hipp, Tai et al. 2017, Li, Stagg et al. 2017, Seckinger, Delgado et al. 2017). However, FcRH5 expression is not limited to tumor plasma cells and B-cell maturation antigen is abundantly secreted in MM patients (Sanchez, Li et al. 2012). A CD38×CD3 bispecific BiTE (Bi38-3) is being tested in clinical trials that consists of two single-chain variable fragments derived from mouse hybridomas, targeting human CD38 and CD3e (Fayon, Martinez-Cingolani et al. 2021).

There are two other CD38×CD3 BsAbs, namely AMG424 and GBR1342, for MM and solid cancers (Jiao, Yi et al. 2020). Currently, they are both based on the structure of Fab-Fc (G1)×scFv-Fc (G1) with a hetero-Fc domain lack of FcγR receptor and complement binding (Drent, Groen et al. 2016). The modified-Fc domain results in the deficiency of classic Fc-dependent immune effector mechanisms. Antigen-independent cytokine release syndrome (CRS) might occur if the Fc regions of BsAbs bind FcγR on T cells, which may cause nonspecific activation of T cells (Chatenoud, Ferran et al. 1990). These two features may limit the specificity or efficiency of these cognate bispecific antibodies in vivo.

To prevent the off-target toxicity, Fc domain mutations were added to bispecific T cell engagers to improve T-cell trafficking and antitumor potency (Wang, Hoseini et al. 2019). Recently, an anti-CD38 bispecific antibody, AMG424, was shown to eliminate MM cells in preclinical models, but also to trigger “off tumor” T-cell cytotoxicity on B, T, and NK cells in vitro (Munoz, Mittelbrunn et al. 2008, Zuch de Zafra, Fajardo et al. 2019). Thus, development of an efficient and safer bispecific antibody could contribute to improving the treatment of MM and other cancers. In order to minimize on-target toxicity, BsAb Fc-mediated immune functions are unwanted. In order to avoid or reduce antigen-independent cytokine release syndrome (CRS) due to crosslinking of CD3 and Fcγ receptors followed by nonspecific activation of immune cells, mutations have been introduced into Fc domain to eliminate FcγR binding.

CD38×CD3 BsAbs by Amgen also demonstrated a positive correlation between CD3 affinity and cytokine release syndrome (CRS). In Amgen's case, three BsAbs: XmAb4, AMG424, and XmAb5, were constructed with the same affinity to CD38, but with varying dissociation constant Kd values with levels of 4.4, 34, and 150-230 nM of Kd to CD3, respectively. The CRS side effects were studied comparatively in cynomolgus monkeys for lead selection. XmAb4 and XmAb5 were not tolerated well in monkeys since dosing with both mAbs resulted in high levels of CRS effects. Thus, AMG424 with moderate affinity to CD3 was selected for further clinical development highlighting the importance of balancing efficacy and safety for these classes of molecules.

Numerous reports on the limitations of CD3+ T-cell redirection anti-tumor efficacy point to the recruitment of counterproductive CD3+ T-cell subsets, dose-limiting cytokine storm, the presence of an immunosuppressive tumor microenvironment (TME), T-cell dysfunction and exhaustion due to expression of immune checkpoint molecules, tumor antigen escape, on-target off-tumor toxicity, and suboptimal potency. In May of 2021, Pfizer's elranatamab (BCMA×CD3) pivotal clinical trial on MM was put on clinical hold due to several cases of severe peripheral neuropathy (Biopharmadive.com 2021). In fact, safety rather than efficacy is often a key concern of T-cell redirection as reflected by the relatively limited commercial success of Catumaxomab (voluntary market withdrawal in the US in 2013 and in EU in 2017) and Blinatumomab (2020 global sales of only 379 MM USD despite regulatory approval since 2014) (Amgen 2021, Wikipedia 2021). Thus, increasing the therapeutic index of T-cell redirection to maximize the clinical potential is an unmet medical need and highly desirable. Therefore, modulating T-cell activation by attenuating CD3-targeted binding affinity while maintaining anti-tumor activity is a promising method to improve the therapeutic window of T-cell engager BsAbs.

Like bispecific T cell engagers, chimeric antigen receptor T-cell immunotherapy (CAR-T) can direct cytotoxic T cells to malignant cells that express specific antigen and subsequently, these T cells activate, proliferate, and release cytokines to lyse tumor cells (Wang, Kaur et al. 2019). Following the fast development of CAR-T technology and the effective outcome of daratumumab and isatuximab in the clinic, CD38 has been a target of chimeric antigen receptors redirected T cells (CAR-T cells) for MM (Wu, Zhang et al. 2019). Preclinical data of CD38-CAR-T cells showed a significant effect on eliminating MM cells in vitro and in vivo and primary malignant cells isolated from patients with MM in vitro. Interestingly, the original CD38 expression disappeared after the treatment with CD38-CAR-T cells (Drent, Groen et al. 2016). Some studies have revealed that high-affinity CD38-CAR-T cells give rise to a strong killing effect not only on myeloma cells but also on normal hematopoietic cells expressing CD38 (Chmielewski, Hombach et al. 2004, Drent, Themeli et al. 2017). To reduce off-tumor CAR responses, CD38-CAR-T cells must be optimized for binding affinity (Drent, Themeli et al. 2017, Yu, Yi et al. 2019). A limitation on the use of CD38-specific CAR-T cells may be represented by a possible toxicity of this approach, due to the presence of CD38 on normal cells such as NK cells, activated T cells, and B cells. Another limitation is represented by the variable expression of CD38 on cancer cells. Besides, it is more desirable and convenient to have an off-the-shelf drug to increase accessibility of T cell therapy.

While BsAbs generated from antibody fragments suffer biophysical and pharmacokinetic hurdles, a drawback of those built with full length antibody-like formats is that they engage co-target antigens multivalently in the absence of the primary target antigen, leading to nonspecific activation and potentially toxicity. The present disclosure provides anti-CD38 and anti-CD3 antibodies including bispecific antibodies directed to CD3 and CD38 to address one or more drawbacks of the existing therapies.

### SUMMARY OF THE DISCLOSURE

In one aspect, the present disclosure provides an anti-CD38 antibody or an antigen-binding portion thereof. In some embodiments, the present disclosure provides an anti-CD38 antibody or an antigen-binding portion thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 64, 65, and 66;
SEQ ID NOs: 64, 83, and 84;
SEQ ID NOs: 64, 65, and 85; and
SEQ ID NOs: 64, 65, and 86; respectively.

In some embodiments, the present disclosure provides an anti-CD38 antibody or an antigen-binding portion thereof, comprising a light chain variable region comprising three CDRs, designated as LCDR1, LCDR2, and LCDR3, wherein the LCDR1, LCDR2, and LCDR3 are selected from:
SEQ ID NOs: 61, 62, and 63;
SEQ ID NOs: 76, 77, and 78;
SEQ ID NOs: 79, 80, and 81; and
SEQ ID NOs: 82, 62, and 78; respectively.

In some embodiments, the disclosure provides an anti-CD38 antibody or antigen binding fragment thereof, comprising a heavy chain sequence comprising an amino acid sequence with at least 85% identity to any one of SEQ ID NOs: 12-16 or an antigen-binding portion thereof, and a light chain sequence comprising an amino acid sequence with at least 85% identity to any one of SEQ ID NOs: 7-11 or an antigen-binding portion thereof.

In some embodiments, the disclosure provides an anti-CD38 antibody or antigen binding fragment thereof, comprising a light chain sequence and a heavy chain sequence comprising: SEQ ID NOs: 2 and 3; SEQ ID NOs: 7 and 12; SEQ ID NOs: 7 and 13; SEQ ID NOs: 7 and 14; SEQ ID NOs: 7 and 15; SEQ ID NOs: 7 and 16; SEQ ID NOs: 8 and 12; SEQ ID NOs: 8 and 13; SEQ ID NOs: 8 and 14; SEQ ID NOs: 8 and 15; SEQ ID NOs: 8 and 16; SEQ ID NOs: 9 and 12; SEQ ID NOs: 9 and 13; SEQ ID NOs: 9 and 14; SEQ ID NOs: 9 and 15; SEQ ID NOs: 9 and 16; SEQ ID NOs: 10 and 12; SEQ ID NOs: 10 and 13; SEQ ID NOs: 10 and 14; SEQ ID NOs: 10 and 15; SEQ ID NOs: 10 and 16; SEQ ID NOs: 11 and 12; SEQ ID NOs: 11 and 13; SEQ ID NOs: 11 and 14; SEQ ID NOs: 11 and 15; or SEQ ID NOs: 11 and 16; respectively.

In some embodiments, the present disclosure provides an anti-CD38 antibody or an antigen-binding portion thereof, comprising at least one variable-heavy-chain-only single-domain or an antigen-binding portion thereof, wherein the at least one variable-heavy-chain-only single-domain comprises HCDR1, HCDR2, and HCDR3 selected from:
SEQ ID NOs: 67, 68, and 69;
SEQ ID NOs: 67, 87, and 69;
SEQ ID NOs: 67, 88, and 69;
SEQ ID NOs: 67, 89, and 69;
SEQ ID NOs: 67, 68, and 90;
SEQ ID NOs: 70, 91, and 72;
SEQ ID NOs: 70, 92, and 72;
SEQ ID NOs: 70, 93, and 72;
SEQ ID NOs: 94, 95, and 96;
SEQ ID NOs: 70, 71, and 97;
SEQ ID NOs: 73, 98, and 75;
SEQ ID NOs: 73, 99, and 75;
SEQ ID NOs: 73, 100, and 75;
SEQ ID NOs: 73, 101, and 102; and
SEQ ID NOs: 73, 103, and 104; respectively.

In some embodiments, the present disclosure provides an anti-CD38 antibody or an antigen-binding portion thereof, comprising at least one variable-heavy-chain-only single-domain or an antigen-binding portion thereof, wherein the at least one variable-heavy-chain-only (VHO) single-domain comprises an amino acid sequence with at least 85% identity to any one of SEQ ID NOs: 4 and 17-30 or an antigen-binding portion thereof.

In another aspect, the present disclosure provides an anti-CD3 antibody or an antigen-binding portion thereof. In some embodiments, the present disclosure provides an anti-CD3 antibody or an antigen-binding portion thereof comprising a heavy chain variable region comprising three Complementarity Determining Regions (CDRs), designated as HCDR1, HCDR2, and HCDR3, wherein the HCDR1, HCDR2, and HCDR3 are selected from:
SEQ ID NOs: 105, 108, and 107;
SEQ ID NOs: 105, 109, and 107;
SEQ ID NOs: 105, 110, and 107; and
SEQ ID NOs: 118, 119, and 120; respectively.

In some embodiments, the present disclosure provides an anti-CD3 antibody or an antigen-binding portion thereof, comprising a light chain variable region comprising three CDRs, designated as LCDR1, LCDR2, and LCDR3, wherein the LCDR1, LCDR2, and LCDR3 are selected from:
SEQ ID NOs: 114, 112, and 113; and
SEQ ID NOs: 115, 116, and 117; respectively.

In some embodiments, the disclosure provides an anti-CD3 antibody or antigen binding fragment thereof comprising a heavy chain sequence comprising an amino acid sequence with at least 85% identity to any one of SEQ ID NOs: 32-34 and 39 or an antigen-binding portion thereof, and a light chain sequence comprising an amino acid sequence with at least 85% identity to any one of SEQ ID NOs: 36-38 or an antigen-binding portion thereof.

In some embodiments, the disclosure provides an anti-CD3 antibody or antigen binding fragment thereof, comprising a light chain sequence and a heavy chain sequence comprising: SEQ ID NOs: 39 and 38; SEQ ID NOs: 41 and 40; SEQ ID NOs: 31 and 35; SEQ ID NOs: 31 and 36; SEQ ID NOs: 31 and 37; SEQ ID NOs: 32 and 35; SEQ ID NOs: 32 and 36; SEQ ID NOs: 32 and 37; SEQ ID NOs: 33 and 35; SEQ ID NOs: 33 and 36; SEQ ID NOs: 33 and 37; SEQ ID NOs: 34 and 35; SEQ ID NOs: 34 and 36; or SEQ ID NOs: 34 and 37; respectively.

The anti-CD38 or anti-CD3 antibodies or antigen binding fragments thereof disclosed herein may be human, humanized, or chimeric antibodies, or antigen binding fragments.

The anti-CD38 or anti-CD3 antibodies or antigen binding fragments thereof disclosed herein may be full length IgG1, IgG2, IgG3, or IgG4 antibodies or may be antigen-binding fragments thereof, such as a Fab, F(ab′)2, or scFv fragment. The antibody backbones may be modified to affect functionality, e.g., to eliminate residual effector functions.

In another aspect, the present disclosure provides a bispecific antibody comprising:
a first binding arm comprising:
a first heavy chain fusion protein comprising, from the N- to the C-terminus, a shield A, a protease sequence A, and an IgG heavy chain or an antigen-binding portion thereof, and
a first light chain fusion protein comprising, from the N- to the C-terminus, a shield B, a protease sequence B, and an IgG light chain or an antigen-binding portion thereof,
wherein the IgG heavy chain or an antigen-binding portion thereof and the IgG light chain or an antigen-binding portion thereof of the first binding arm can target a CD3 associated pathway and comprise an anti-CD3 antibody or antigen binding fragment as described herein; and
a second binding arm comprising:
a second heavy chain fusion protein comprising, from the N- to the C-terminus, a shield C, a protease sequence C, and an IgG heavy chain or an antigen-binding portion thereof; and
a second light chain fusion protein comprising, from the N- to the C-terminus, a shield D, a protease sequence D, and an IgG light chain or an antigen-binding portion thereof,
wherein the IgG heavy chain or an antigen-binding portion thereof and the IgG light chain or an antigen-binding portion thereof of the second binding arm can target a CD38 associated pathway and comprise an anti-CD38 antibody or antigen binding fragment as described herein.
   The shields A-D can be the same or different from one another, and the protease sequences A-D can be the same or different from one another. The shields A-D and the protease sequences A-D are optional, meaning that some or all of them may be present or absent.

In certain embodiments, the present disclosure provides a bispecific antibody comprising:
a first binding arm comprises:
a first heavy chain fusion protein comprising, from the N- to the C-terminus, a signal sequence A-a shield A-a linker A-a protease sequence A-a linker B-an IgG heavy chain or an antigen-binding portion thereof, and
a first light chain fusion protein comprising, from the N- to the C-terminus, a signal sequence B-a shield B-a linker C-a protease sequence B-a linker D-an IgG light chain or an antigen-binding portion thereof,
wherein the IgG heavy chain or an antigen-binding portion thereof and the IgG light chain or an antigen-binding portion thereof of the first binding arm are capable of targeting a CD3 associated pathway and comprise an anti-CD3 antibody or antigen binding fragment as described herein; and
the second binding arm comprises:
a second heavy chain fusion protein comprising, from the N- to the C-terminus, a signal sequence C-a shield C-a linker E-a protease sequence C-a linker F-an IgG heavy chain or an antigen-binding portion thereof; and
a second light chain fusion protein comprising, from the N- to the C-terminus, a signal sequence D-a shield D-a linker G-a protease sequence D-a linker H-an IgG light chain or an antigen-binding portion thereof,
wherein the IgG heavy chain or an antigen-binding portion thereof and the IgG light chain or an antigen-binding portion thereof of the second binding arm are capable of targeting a CD38 associated pathway and comprise an anti-CD38 antibody or antigen binding fragment as described herein.
   The signal sequences A-D can be the same or different from one another, and the linkers A-H can be the same or different from one another. The signal sequences A-D, shields A-D, protease sequences A-D, and linkers A-H are optional, meaning that some or all of them may be present or absent.

In another aspect, the present disclosure provides a bispecific antibody comprising:
a first binding arm comprising:
a first heavy chain fusion protein comprising, from the N- to the C-terminus, a shield A, a protease sequence A, and an IgG heavy chain or an antigen-binding portion thereof, and
a first light chain fusion protein comprising, from the N- to the C-terminus, a shield B, a protease sequence B, and an IgG light chain or an antigen-binding portion thereof,
wherein the IgG heavy chain or an antigen-binding portion thereof and the IgG light chain or an antigen-binding portion thereof of the first binding arm are capable of targeting a CD3 associated pathway and comprise an anti-CD3 antibody or antigen binding fragment as described herein; and
a second binding arm comprising:
a second heavy chain fusion protein comprising, from the N- to the C-terminus, a shield C, a protease sequence C, and an IgG heavy chain comprising at least one variable-heavy-chain-only (VHO) single domain or an antigen-binding portion thereof,
wherein the at least one variable-heavy-chain-only single domain or an antigen-binding portion thereof is capable of targeting a CD38 associated pathway and comprises an anti-CD38 VHO or antigen binding fragment as described herein.
   The shields A-C can be the same or different from one another, and the protease sequences A-C can be the same or different from one another. The shields A-C and the protease sequences A-C are optional, meaning that some or all of them may be present or absent.

In certain embodiments, the present disclosure provides a bispecific antibody comprising:
a first binding arm comprises:
a first heavy chain fusion protein comprising, from the N- to the C-terminus, a signal sequence A-a shield A-a linker A-a protease sequence A-a linker B-an IgG heavy chain or an antigen-binding portion thereof, and
a first light chain fusion protein comprising, from the N- to the C-terminus, a signal sequence B-a shield B-a linker C-a protease sequence B-a linker D-an IgG light chain or an antigen-binding portion thereof,
wherein the IgG heavy chain or an antigen-binding portion thereof and the IgG light chain or an antigen-binding portion thereof of the first binding arm are capable of targeting a CD3 associated pathway; and
the second binding arm comprises:
a second heavy chain fusion protein comprising, from the N- to the C-terminus, a signal sequence C-a shield C-a linker E-a protease sequence C-a linker F-an IgG heavy chain comprising at least one variable-heavy-chain-only single-domain or an antigen-binding portion thereof,
wherein the at least one variable-heavy-chain-only single-domain or an antigen-binding portion thereof is capable of targeting a CD38 associated pathway and comprises an anti-CD38 VHO or antigen binding fragment as described herein.
   The signal sequences A-C can be the same or different from one another, and the linkers A-F can be the same or different from one another. The signal sequences A-C, shields A-C, protease sequences A-C, and linkers A-F are optional, meaning that some or all of them may be present or absent.

In certain embodiments, in a bispecific antibody disclosed herein, the IgG is human IgG1, IgG2, IgG3, or IgG4.

In certain embodiments, in a bispecific antibody disclosed herein, the first binding arm is monovalent, and the second binding arm is monovalent, bivalent, or multivalent.

In certain embodiments, in a bispecific antibody disclosed herein, the second binding arm comprises two or three IgG variable-heavy-chain-only single domains in tandem, wherein the two or three IgG variable-heavy-chain-only single domains are optionally connected via one or more linker sequences.

In certain embodiments, in a bispecific antibody disclosed herein, the first binding arm (anti-CD3 arm) comprises:
a heavy chain variable region comprising three Complementarity Determining Regions (CDRs), designated as HCDR1, HCDR2, and HCDR3, wherein the HCDR1, HCDR2, and HCDR3 are selected from: SEQ ID NOs: 105, 108, and 107; SEQ ID NOs: 105, 109, and 107; SEQ ID NOs: 105, 110, and 107; and SEQ ID NOs: 118, 119, and 120; respectively; and
a light chain variable region comprising three CDRs, designated as LCDR1, LCDR2, and LCDR3, wherein the LCDR1, LCDR2, and LCDR3 are selected from: SEQ ID NOs: 114, 112, and 113; and SEQ ID NOs: 115, 116, and 117; respectively.

In certain embodiments, in a bispecific antibody disclosed herein, the second binding arm (anti-CD38 arm) comprises:
a heavy chain variable region comprising three Complementarity Determining Regions (CDRs), designated as HCDR1, HCDR2, and HCDR3, wherein the HCDR1, HCDR2, and HCDR3 are selected from: SEQ ID NOs: 64, 65, and 66; SEQ ID NOs: 64, 83, and 84; SEQ ID NOs: 64, 65, and 85; and SEQ ID NOs: 64, 65, and 86; respectively; and a light chain variable region comprising three CDRs, designated as LCDR1, LCDR2, and LCDR3, wherein the LCDR1, LCDR2, and LCDR3 are selected from: SEQ ID NOs: 61, 62, and 63; SEQ ID NOs: 76, 77, and 78; SEQ ID NOs: 79, 80, and 81; and SEQ ID NOs: 82, 62, and 78; respectively.

In certain embodiments, in a bispecific antibody disclosed herein, the second binding arm (anti-CD38 arm) comprises at least one variable-heavy-chain-only single-domain or an antigen-binding portion thereof, wherein the at least one variable-heavy-chain-only single-domain comprises three CDRs (HCDR1, HCDR2, and HCDR3) selected from: SEQ ID NOs: 67, 68, and 69; SEQ ID NOs: 67, 87, and 69; SEQ ID NOs: 67, 88, and 69; SEQ ID NOs: 67, 89, and 69; SEQ ID NOs: 67, 68, and 90; SEQ ID NOs: 70, 91, and 72; SEQ ID NOs: 70, 92, and 72; SEQ ID NOs: 70, 93, and 72; SEQ ID NOs: 94, 95, and 96; SEQ ID NOs: 70, 71, and 97; SEQ ID NOs: 73, 98, and 75; SEQ ID NOs: 73, 99, and 75; SEQ ID NOs: 73, 100, and 75; SEQ ID NOs: 73, 101, and 102; and SEQ ID NOs: 73, 103, and 104; respectively.

In certain embodiments, in a bispecific antibody disclosed herein, the IgG heavy chain of the first binding arm (anti-CD3 arm) comprises an amino acid sequence selected from SEQ ID NO: 31-34, 39, and 41, an amino acid sequence having at least 85% identity to any one of SEQ ID NO: 31-34, 39, and 41, or an antigen-binding portion thereof, and the IgG light chain of the first binding arm comprises an amino acid sequence selected from SEQ ID NO: 35-38 and 40, an amino acid sequence having at least 85% identity to any one of SEQ ID NO: 35-38 and 40, or antigen-binding portion thereof.

In certain embodiments, in a bispecific antibody disclosed herein, the IgG heavy chain of the second binding arm (anti-CD38 arm) comprises an amino acid sequence selected from SEQ ID NOs: 3-6 and 12-30, an amino acid sequence having at least 85% identity to any one of SEQ ID NOs: 3-6 and 12-30, or an antigen-binding portion thereof, and the IgG light chain of the second binding arm comprises an amino acid sequence selected from SEQ ID NOs: 2 and 7-11, an amino acid sequence having at least 85% identity to any one of SEQ ID NOs: 2 and 7-11, or an antigen-binding portion thereof.

In certain embodiments, in a bispecific antibody disclosed herein, the at least one variable-heavy-chain-only single domain comprises an amino acid sequence selected from SEQ ID NOs: 4-6 and 17-30, an amino acid sequence having at least 85% identity to any one of SEQ ID NOs: 4-6 and 17-30, or an antigen-binding portion thereof.

In certain embodiments, in a bispecific antibody disclosed herein, the shield A, shield B, shield C, and shield D are each independently selected from the amino acid sequences set forth in SEQ ID NOs: 42-52.

In certain embodiments, in a bispecific antibody disclosed herein, the protease sequence A, protease sequence B, protease sequence C, and protease sequence D are each independently selected from amino acid sequences set forth in SEQ ID NOs: 53-60.

In certain embodiments, in a bispecific antibody disclosed herein, the IgG heavy chain and IgG light chain of the first binding arm (anti-CD3 arm) respectively comprise the amino acid sequences set forth in SEQ ID NOs: 39 and 38; SEQ ID NOs: 41 and 40; SEQ ID NOs: 31 and 35; SEQ ID NOs: 31 and 36; SEQ ID NOs: 31 and 37; SEQ ID NOs: 32 and 35; SEQ ID NOs: 32 and 36; SEQ ID NOs: 32 and 37; SEQ ID NOs: 33 and 35; SEQ ID NOs: 33 and 36; SEQ ID NOs: 33 and 37; SEQ ID NOs: 34 and 35; SEQ ID NOs: 34 and 36; or SEQ ID NOs: 34 and 37.

In certain embodiments, in a bispecific antibody disclosed herein, the IgG light chain and IgG heavy chain of the second binding arm (anti-CD38 arm) respectively comprise the amino sequences set forth in SEQ ID NOs: 2 and 3; SEQ ID NOs: 7 and 12; SEQ ID NOs: 7 and 13; SEQ ID NOs: 7 and 14; SEQ ID NOs: 7 and 15; SEQ ID NOs: 7 and 16; SEQ ID NOs: 8 and 12; SEQ ID NOs: 8 and 13; SEQ ID NOs: 8 and 14; SEQ ID NOs: 8 and 15; SEQ ID NOs: 8 and 16; SEQ ID NOs: 9 and 12; SEQ ID NOs: 9 and 13; SEQ ID NOs: 9 and 14; SEQ ID NOs: 9 and 15; SEQ ID NOs: 9 and 16; SEQ ID NOs: 10 and 12; SEQ ID NOs: 10 and 13; SEQ ID NOs: 10 and 14; SEQ ID NOs: 10 and 15; SEQ ID NOs: 10 and 16; SEQ ID NOs: 11 and 12; SEQ ID NOs: 11 and 13; SEQ ID NOs: 11 and 14; SEQ ID NOs: 11 and 15; or SEQ ID NOs: 11 and 16.

In certain embodiment, in a bispecific antibody disclosed herein, the at least one variable-heavy-chain-only single domain comprises two variable-heavy-chain-only single domains each independently selected from SEQ ID NO: 4-6 and 17-30.

In certain embodiments, in a bispecific antibody disclosed herein, the at least one variable-heavy-chain-only single domain comprises, from the N- to the C-terminus, SEQ ID NO: 4 and SEQ ID NO: 5 optionally connected via a linker, SEQ ID NO: 4 and SEQ ID NO: 6 optionally connected via a linker, SEQ ID NO: 5 and SEQ ID NO: 6 optionally connected via a linker, SEQ ID NO: 5 and SEQ ID NO: 4 optionally connected via a linker, SEQ ID NO: 6 and SEQ ID NO: 5 optionally connected via a linker, or SEQ ID NO: 6 and SEQ ID NO: 4 optionally connected via a linker.

In certain embodiments, an antibody or bispecific antibody disclosed herein comprises a modified Fc to extend the half-life of the bispecific antibody, enhance resistance of the bispecific antibody to proteolytic degradation, reduce effector functionality of the bispecific antibody, facilitate generation of the bispecific antibody by Fc heterodimerization, facilitate multimerization of the bispecific antibody, and/or improve manufacturing and drug stability of the bispecific antibody.

In another aspect, the present disclosure provides a conjugate comprising an antibody or bispecific antibody disclosed herein conjugated to a moiety, such as a cytotoxic agent.

In another aspect, the present disclosure provides a composition comprising an antibody or bispecific antibody disclosed herein or a conjugate disclosed herein. In certain embodiments, the present disclosure provides a pharmaceutical composition comprising an antibody or bispecific antibody disclosed herein or a conjugate disclosed herein, and a pharmaceutically acceptable carrier.

In another aspect, the present disclosure provides a nucleic acid encoding an anti-CD38 antibody or an antigen-binding portion thereof.

In another aspect, the present disclosure provides a nucleic acid encoding an anti-CD3 antibody or an antigen-binding portion thereof.

In another aspect, the present disclosure provides a nucleic acid encoding the bispecific antibody, the first heavy chain fusion protein, the first light chain fusion protein, the second heavy chain fusion protein, or the second light chain fusion protein disclosed herein.

In another aspect, the present disclosure provides a recombinant vector, such as an expression vector, comprising a nucleic acid disclosed herein.

In another aspect, the present disclosure provides a host cell comprising a recombinant vector such as an expression vector or a nucleic acid disclosed herein.

In another aspect, the present disclosure provides a method for preparing an antibody or bispecific antibody disclosed herein, comprising culturing a host cell disclosed herein, growing the host cell in a host cell culture, providing host cell culture conditions wherein a nucleic acid disclosed herein is expressed, and recovering the antibody or bispecific antibody from the host cell or from the host cell culture. In certain embodiments, the bispecific antibody is obtained using controlled Fab arm exchange.

In another aspect, the present disclosure provides a method for treating or preventing a CD38-mediated disease or disorder in a subject in need thereof, comprising administering to the subject a pharmaceutically effective amount of an antibody, bispecific antibody, conjugate, or a pharmaceutical composition disclosed herein. In certain embodiments, the disease or disorder is selected from human cancers, including gastric and colorectal cancer, pancreatic cancer, prostate cancer, lung cancer, hepatocellular cancer, triple-negative breast cancer, nasopharyngeal cancer, cervical cancer, hematologic malignancies such as MM, lymphoma, acute myeloid leukemia, chronic lymphocytic leukemia, acute B lymphoblastic leukemia, and others, heart diseases, viral infection including HIV infection, asthma and other respiratory inflammatory disorders, allergic airway disease, fetomaternal tolerance, autism spectrum disorders, glomerular sclerosis, inflammatory bowel disease, rheumatoid arthritis, diabetes mellitus, diabetes, chronic autoimmune thyroiditis and Graves' disease, neurodegenerative, and neuro-inflammatory diseases, such as Alzheimer's Disease. In some embodiments, the CD38-mediated disease or disorder is selected from human cancers, including gastric and colorectal cancer, pancreatic cancer, prostate cancer, lung cancer, hepatocellular cancer, triple-negative breast cancer, nasopharyngeal cancer, cervical cancer, and hematologic malignancies.

In another aspect, the present disclosure provides a method for mediating CD38 in a subject in need thereof, comprising administering to the subject an effective amount of an antibody or bispecific antibody or a pharmaceutical composition disclosed herein.

These and other embodiments of the present disclosure are described in greater detail herein.

### BRIEF DESCRIPTION OF THE DRAWINGS

FIG. 1 is a schematic diagram illustrating the roles of CD38 in immune cells.

FIG. 1A shows that CD38 is predominantly expressed on immune cells and metabolizes nicotinamide nucleotides (NAD⁺ and NMN) to ADPR and cADPR (Hogan, Chini et al. 2019).

FIG. 1B shows multiple immunosuppressive mechanisms that impede anti-tumor immunity.

FIG. 2 shows mRNA expression profiles of CD38.

FIG. 3 illustrates mechanisms of action of an anti-CD38 antibody.

FIG. 4 illustrates mechanisms of resistance to anti-CD38 antibodies.

FIG. 5 is a schematic drawing of a CD38×CD3 bispecific antibody. FIG. 5A shows a shielded bispecific antibody that comprises two different sets of heavy chain (HC) and light chain (LC) pairing as indicated under the notations of “first arm” and “second arm.” Both arms can be shielded. FIG. 5B shows a bispecific antibody that comprises two different sets of heavy chain (HC) and light chain (LC) pairing without the shielding or masking domains. FIG. 5C illustrates certain components of the CD3 arm (first arm) open reading frame and the CD38 binding arm (second arm) open reading frame with the shielding. FIG. 5D illustrates certain components of the CD3 arm (first arm) open reading frame and the CD38 binding arm (second arm) open reading frame without the shielding.

FIG. 6 is a schematic diagram of a CD3×CD38 bispecific antibody comprising one or more anti-CD38 variable-heavy-only (VHO) single domains. FIG. 6A shows two examples of such a CD3×CD38 bispecific antibody: a bispecific antibody comprising a single VHO in the “second arm,” an arm targeting CD38 (left panel); a bispecific antibody comprising a CD38 binding arm (second arm) that comprises two VHOs fused together (right panel). In both examples, the VHOs are connected to a masking domain.

FIG. 6B shows, in the left panel, a bispecific antibody comprising a single VHO in the “second arm,” an arm targeting CD38; and in the right panel, a bispecific antibody comprising a CD38 binding arm (second arm) that comprises two VHOs fused together. In both examples, the VHOs are not connected with a masking domain.

FIG. 6C illustrates certain components of the CD3 arm (first arm) open reading frame and the CD38 binding arm (second arm) open reading frame with the shielding.

FIG. 6D illustrates certain components of the CD3 arm (first arm) open reading frame and the CD38 binding arm (second arm) open reading frame without the shielding.

FIG. 7 is a schematic representation of a protease digestion removal of the masking domains from a CD38×CD3 bispecific antibody.

FIG. 8 demonstrated concentration dependent ELISA binding of anti-CD3 antibodies to recombinant human (FIG. 8A) and cynomolgus monkey (FIG. 8B) CD3 delta and epsilon domain protein in an ELISA assay. The positive control anti-CD3 antibody, SP34, was used in the binding assay. The SP34 has a heavy chain variable domain sequence: EVOLVESGGGLVQPGGSLKLSCAASGFTFNTYAMNWVRQAPGKGLEWVARIRSKYNN YATYYADSVKDRFTISRDDSKNTAYLQMNNLKTEDTAVYYCVRHGNFGNSYVSWFAY WGQGTLVTVSS (SEQ ID NO: 127) and a light chain variable domain sequence: QTVVTQEPSLTVSPGGTVTLTCRSSTGAVTTSNYANWVQQKPGQAPRGLIGGTNKRAP GTPARFSGSLLGGKAALTLSGVQPEDEAEYYCALWYSNLWVFGGGTKLTVL (SEQ ID NO: 38). The y axis is the binding response expressed in optical density at 450 nm absorbance units. The x axis is the concentration of the molecules tested. SP34 and SP34 v8 (SEQ ID NO: 38 as light chain and SEQ ID NO: 39 as heavy chain) had potent binding to human and cynomolgus delta and epsilon domain protein.

FIG. 9 demonstrated concentration dependent flow cytometry binding of anti-CD3 antibodies to primary human T cells bearing CD3 from a PBMC preparation. The y axis is the binding response expressed in global mean fluorescence intensity (gMFI from detecting AlexaFluor647). The x axis is the concentration of the molecules assessed.

FIG. 10 demonstrated concentration dependent ELISA binding of anti-CD38 antibodies to (FIGS. 10A, D, and E) recombinant human, (FIG. 10B) recombinant cynomolgus monkey, and (FIG. 10C) mouse recombinant CD38 protein. The concentration dependent ELISA binding of anti-CD38 single domain antibodies to human CD38 are shown in FIGS. 10D and E. The y axes were the binding responses expressed in optical density at 450 nm absorbance units. The x axis was the concentration of the molecules tested. The results showed that the molecules with SEQ ID NOs: 2 and 3 (mAb), as well as SEQ ID NOs: 4, 5, 6, 17, 18, 20, 21, 24, and 29 (VHOs), respectively, bound to recombinant human CD38 protein. Molecules with SEQ ID NOs: 4 and 5, respectively, could bind to recombinant cynomolgus monkey CD38 protein. Molecules comprising SEQ ID NO: 4 could bind to recombinant mouse CD38 protein.

FIG. 11 demonstrated dose responses of CD38×CD3 bispecific antibody T cell activation from a CD3-bearing Jurkat T cell reporter assay line in the presence of the CD38 bearing H929 multiple myeloma cells. The CD38 VHO with SEQ ID NO: 4 was paired with different CD3 arms to make the indicated CD38×CD3 bispecific antibodies (denoted as SEQ ID NO: 4×Cris7 v4 (heavy chain variable sequence with SEQ ID NO: 33, light chain variable sequence with SEQ ID NO: 36), SEQ ID NO: 4×Cris7 v3 (heavy chain variable sequence with SEQ ID NO: 32, light chain variable sequence with SEQ ID NO: 37), and SEQ ID NO: 4×SP34 v8 (heavy chain variable sequence with SEQ ID NO: 39, light chain variable sequence with SEQ ID NO: 38), respectively). The y axis is the reporter assay response in relative light units. The x axis is the concentration of the molecules tested.

FIG. 12 demonstrated dose responses of CD38×CD3 bispecific antibody T cell activation from a CD3-bearing Jurkat T cell reporter assay line in the presence of the CD38 bearing L363 multiple myeloma cells. The CD38 VHO with SEQ ID NO: 4 was paired with different CD3 arms to make the indicated CD38×CD3 bispecific antibodies (denoted as SEQ ID NO: 4×Cris7 v3 (heavy chain variable sequence with SEQ ID NO:32, light chain variable sequence with SEQ ID NO: 37), SEQ ID NO: 4×Cris7 v4 (heavy chain variable sequence with SEQ ID NO: 33, light chain variable sequence with SEQ ID NO: 36), and SEQ ID NO: 4×SP34, respectively). The y axis is the reporter assay response in relative light units. The x axis is the concentration of the molecules tested.

FIG. 13 demonstrated dose responses of CD38×CD3 bispecific antibody T cell activation from a CD3-bearing Jurkat T cell reporter assay line in the presence of the CD38 bearing RPMI 8226 multiple myeloma cells. The CD38 VHO with SEQ ID NO: 4 was paired with different CD3 arms to make the indicated CD38×CD3 bispecific antibodies. Potent T cell activation responses were observed. The y axis is the reporter assay response in relative light units. The x axis is the concentration of the molecules assessed.

FIG. 14 demonstrated dose responses of CD38×CD3 bispecific antibody directed primary human T cell killing of H929, a CD38 bearing multiple myeloma cell line. Two different lots (FIGS. 14A and 14B) of PBMCs were used. The CD38 VHO with SEQ ID NO: 4 was paired with different CD3 arms to make the CD38×CD3 bispecific antibodies indicated in FIG. 14. Potent T cell killing responses were observed. The y axis is the percent cell killing. The x axis is the concentration of the molecules assessed.

FIG. 15 demonstrated the dose responses of CD38×CD3 bispecific antibody directed primary human T cell killing of RPMI 8226, a CD38 bearing multiple myeloma cell line. Four different lots (FIGS. 15A, 15B, 15C, and 15D) of PBMCs were used. The CD38 VHO with SEQ ID NO: 4 was paired with different CD3 arms to make the CD38×CD3 bispecific antibodies indicated in FIG. 15. Potent T cell killing responses were observed. The y axis is the percent cell killing. The x axis is the concentration of the molecules assessed.

FIG. 16 demonstrated the dose responses of CD38×CD3 bispecific antibody directed primary human T cell killing of L363, a CD38 bearing multiple myeloma cell line. Different lots (FIGS. 16A, 16B, 16C, and 16D) of PBMCs were used. The CD38 VHO with SEQ ID NO: 4 was paired with different CD3 arms to make the CD38×CD3 bispecific antibodies indicated in FIG. 16. Potent T cell killing responses were observed. The y axis is the percent cell killing. The x axis is the concentration of the molecules assessed.

FIG. 17 shows a mouse model of efficacies of two CD38×CD3 bispecific antibodies, denoted as SEQ ID NO:4×SP34 v8 (comprising CD38 SEQ ID NO: 4 and CD3 SP34 v8 (SEQ ID NO: 38 as light chain and SEQ ID NO: 39 as heavy chain)); and SEQ ID NO:4×40G5 (comprising CD38 SEQ ID NO:4 and 40 G5 (SEQ ID NO: 40 as light chain and SEQ ID NO: 41 as heavy chain). FIG. 17A shows two study designs (“Donor 1/2” and “Donor 3/4”) of a mouse efficacy model using transplanted human PBMCs and human NCI-H929 multiple myeloma cells treated with a bispecific antibody comprising a novel anti-CD38 specificity and a positive control anti-CD3 antibody with known anti-CD3 bispecific antibody activity. FIGS. 17B-17C (corresponding to the study design Donor 1/2) and FIGS. 17D-17E (corresponding to the study design Donor 3/4) show the human NCI-H929 multiple myeloma cell tumor growth inhibition in a mouse efficacy model using transplanted human PBMCs and treated with antibodies indicated in FIGS. 17B-17E.

### DETAILED DESCRIPTION OF THE DISCLOSURE

### Definitions

All publications, including but not limited to patents and patent applications, cited in this specification are herein incorporated by reference as though fully set forth. If certain content of a reference cited herein contradicts or is inconsistent with the present disclosure, the present disclosure controls.

Any one embodiment of the disclosure described herein, including those described only in one section of the specification describing a specific aspect of the disclosure, and those described only in the examples or drawings, can be combined with any other one or more embodiment(s), unless explicitly disclaimed or improper.

It is to be understood that the terminology used herein is for the purpose of describing particular embodiments only and is not intended to be limiting. Unless defined otherwise, all technical and scientific terms used herein have the same meaning as commonly understood by one of ordinary skill in the art to which the disclosure pertains.

Although any methods and materials similar or equivalent to those described herein may be used in the practice for testing of the present disclosure, exemplary materials and methods are described herein. In describing and claiming the present disclosure, the following terminology will be used.

As used in this specification and the appended claims, the singular forms “a,” “an,” and “the” include plural referents unless the content clearly dictates otherwise. Thus, for example, reference to “a cell” includes a combination of two or more cells, and the like.

“Antibodies” is meant in a broad sense and includes immunoglobulin molecules including monoclonal antibodies including murine, human, humanized and chimeric monoclonal antibodies, antibody fragments, bispecific or multi-specific antibodies, dimeric, tetrameric or multimeric antibodies, single chain antibodies, domain antibodies and any other modified configuration of the immunoglobulin molecule that comprises an antigen binding site of the required specificity.

“Full length antibody molecules” are comprised of two heavy chains (HC) and two light chains (LC) inter-connected by disulfide bonds as well as multimers thereof (e.g., IgM). Each heavy chain is comprised of a heavy chain variable region (V_{H}) and a heavy chain constant region (comprised of domains C_{H1}, hinge, C_{H2} and C_{H3}). Each light chain is comprised of a light chain variable region (V_{L}) and a light chain constant region (C_{L}). The V_{H} and the V_{L} regions may be further subdivided into regions of hyper variability, termed complementarity determining regions (CDR), interspersed with framework regions (FR). Each V_{H} and V_{L} is composed of three CDRs and four FR segments, arranged from amino-to-carboxyl-terminus in the following order: FR1, CDR1, FR2, CDR2, FR3, CDR3 and FR4.

“Complementarity determining regions (CDR)” are “antigen binding sites” in an antibody. CDRs may be defined using various terms: (i) Complementarity Determining Regions (CDRs), three in the V_{H} (HCDR1, HCDR2, HCDR3) and three in the V_{L} (LCDR1, LCDR2, LCDR3) are based on sequence variability (Wu and Kabat 1970) (Kabat et al., Sequences of Proteins of Immunological Interest, 5th Ed. Public Health Service, National Institutes of Health, Bethesda, Md., 1991). (ii) “Hypervariable regions,” “HVR,” or “HV,” three in the V_{H} (H1, H2, H3) and three in the V_{L} (L1, L2, L3) refer to the regions of an antibody variable domains which are hypervariable in structure as defined by Chothia and Lesk (Chothia and Lesk 1987). The International ImMunoGeneTics (IMGT) database (http://www_imgt_org) provides a standardized numbering and definition of antigen-binding sites. The correspondence between CDRs, HVs, and IMGT delineations are described (Lefranc, Pommie et al. 2003). The term “CDR,” “HCDR1,” “HCDR2,” “HCDR3,” “LCDR1,” “LCDR2” and “LCDR3” as used herein includes CDRs defined by any of the methods described supra, Kabat, Chothia or IMGT, unless otherwise explicitly stated in the specification.

Immunoglobulins may be assigned to five major classes, IgA, IgD, IgE, IgG and IgM, depending on the heavy chain constant region amino acid sequence. IgA and IgG are further sub-classified as the isotypes IgA₁, IgA₂, IgG₁, IgG₂, IgG₃ and IgG₄. Antibody light chains of any vertebrate species may be assigned to one of two clearly distinct types, namely kappa (κ) and lambda (λ), based on the amino acid sequences of their constant regions.

“Antibody fragment,” “antigen binding fragment,” or “antigen binding portion,” refers to a portion of an immunoglobulin molecule that retains the heavy chain and/or the light chain antigen binding site, such as heavy chain complementarity determining regions (HCDR) 1, 2 and 3, light chain complementarity determining regions (LCDR) 1, 2 and 3, a heavy chain variable region (V_{H}), or a light chain variable region (V_{L}). Antibody fragments include well known F_{ab}, F(_{ab′})2, Fa and Fr fragments as well as domain antibodies (dAb) consisting of one V_{H} domain. V_{H} and V_{L} domains may be linked together via a synthetic linker to form various types of single chain antibody designs where the V_{H}/V_{L} domains may pair intramolecularly, or intermolecularly in those cases when the V_{H} and V_{L} domains are expressed by separate single chain antibody constructs, to form a monovalent antigen binding site, such as single chain Fv (scFv) or diabody; described for example in Int. Patent Publ. Nos. WO1998/44001, WO1988/01649, WO1994/13804, and WO1992/01047.

“Antibody mimetic” refers to an engineered antibody protein that exhibits specific binding to a target. For example, antibody mimetic can be an Affibody, a DARPin, an Anticalin, an Avimer, a Versa body, or a Duocalin.

“Monoclonal antibody” refers to an antibody population with single amino acid composition in each heavy and each light chain, except for possible well-known alterations such as removal of C-terminal lysine from the antibody heavy chain. Monoclonal antibodies typically bind one antigenic epitope, except that, e.g., bispecific monoclonal antibodies bind two distinct antigenic epitopes. Monoclonal antibodies may have heterogeneous glycosylation within the antibody population. Monoclonal antibody may be monospecific or multi-specific, or monovalent, bivalent, or multivalent. A bispecific antibody is included in the term monoclonal antibody.

“Isolated antibody” refers to an antibody or antibody fragment that is substantially free of other antibodies having different antigenic specificities. “Isolated antibody” encompasses antibodies that are isolated to a higher purity, such as antibodies that are at least 80%, such as 80%, 81%, 82%, 83%, 84%, 85%, 86%, 87%, 88%, 89%, 90%, 91%, 92%, 93%, 94%, 95%, 96%, 97%, 98%, 99%, or 100% pure.

“Humanized antibody” refers to an antibody in which the antigen binding sites are derived from non-human species and the variable region frameworks are derived from human immunoglobulin sequences. Humanized antibody may include substitutions in the framework so that the framework may not be an exact copy of expressed human immunoglobulin or human immunoglobulin germline gene sequences.

“Human antibody” refers to an antibody having heavy and light chain variable regions in which both the framework and the antigen binding site are derived from sequences of human origin and is optimized to have minimal immune response when administered to a human subject. If the antibody contains a constant region or a portion of the constant region, the constant region also is derived from sequences of human origin.

“Anti-target” refers to an antibody or antibody domain (also referred to as an antigen-binding portion or fragment of an antibody) that can bind to the specified target molecule such as CD38 (i.e., anti-CD38 is an antibody or antibody domain that can bind to CD38). The style “CD38” refers to the CD38 protein or CD38 gene product. The style “CD38” refers to the CD38 gene.

“CD3×CD38” refers to a bispecific antibody or antibody fragments that can bind to CD3 and CD38. The process of making bispecific antibodies requires recombinant modifications to either of parental mAb amino acid sequences. Although the amino acid sequences of the CH1, CL, and Fc domains of each parental mAb may not be the same, there is no significant difference in the binding between the CD3×CD38 and CD38×CD3 bispecific antibodies. The notations of “first arm” and “second arm” herein are arbitrary. For example, in a bispecific antibody disclosed herein, a first arm could target CD3, and a second arm could target CD38; or a first arm could target CD38 and a second arm could target CD3.

The numbering of amino acid residues in the antibody constant region throughout the specification is according to the EU index as described in Kabat et al., Sequences of Proteins of Immunological Interest, 5th Ed. Public Health Service, National Institutes of Health, Bethesda, MD. (1991), unless otherwise explicitly stated.

Conventional one and three-letter amino acid codes are used herein as shown in Table 1.

TABLE 1

| | Three-letter | |
|---|---|---|
| Amino acid | code | One-letter code |
| Alanine | Ala | A |
| Arginine | Arg | R |
| Asparagine | Asn | N |
| Aspartate | Asp | D |
| Cysteine | Cys | C |
| Glutamate | Gln | E |
| Glutamine | Glu | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

The polypeptides, nucleic acids, fusion proteins, and other compositions provided herein may encompass polypeptides, nucleic acids, fusion proteins, and the like that have a recited percent identity to an amino acid sequence or DNA sequence provided herein. The term “identity” refers to a relationship between the sequences of two or more polypeptide molecules or two or more nucleic acid molecules, as determined by aligning and comparing the sequences. “Percent identity,” “percent homology,” “sequence identity,” or “sequence homology” and the like mean the percent of identical residues between the amino acids or nucleotides in the compared molecules and is calculated based on the size of the smallest of the molecules being compared. For example, a sequence A that is “at least 85% identity” to a sequence B means that sequence A comprises at least 85%, e.g., at least 86%, 87%, 88%, 89%, 90%, 91%, 92%, 93%, 94%, 95%, 96%, 97%, 98%, or 99%, identical residues to those of sequence B. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. In calculating percent identity, the sequences being compared are typically aligned in a way that gives the largest match between the sequences.

The constant region sequences of the mammalian IgG heavy chain are designated in sequence as C_{H1}-hinge-C_{H2}-C_{H3}. The “hinge,” “hinge region” or “hinge domain” of an IgG is generally defined as including Glu216 and terminating at Pro230 of human IgG₁ according to the EU Index but functionally, the flexible portion of the chain may be considered to include additional residues termed the upper and lower hinge regions, such as from Glu216 to Gly237 and the lower hinge has been referred to as residues 233 to 239 of the F_{c} region where FcγR binding was generally attributed. Hinge regions of other IgG isotypes may be aligned with the IgG₁ sequence by placing the first and last cysteine residues forming inter-heavy chain S-S bonds. Although boundaries may vary slightly, as numbered according to the EU Index, the C_{H1} domain is adjacent to the V_{H} domain and amino terminal to the hinge region of an immunoglobulin heavy chain molecule and includes the first (most amino terminal) constant region of an immunoglobulin heavy chain, e.g., from about EU positions 118-215. The Fc domain extends from amino acid 231 to amino acid 447; the CH2 domain is from about Ala231 to Lys340 or Gly341 and the CH3 from about Gly341 or Gln342 to Lys447. The residues of the IgG heavy chain constant region of the C_{H1} region terminate at Lys. The F_{c} domain containing molecule comprises at least the C_{H2} and the C_{H3} domains of an antibody constant region, and therefore comprises at least a region from about Ala231 to Lys447 of IgG heavy chain constant region. The F_{c} domain containing molecule may optionally comprise at least portion of the hinge region.

“Epitope” refers to a portion of an antigen (e.g., CD3 or CD38) to which an antibody specifically binds. Epitopes typically consist of chemically active (such as polar, non-polar, or hydrophobic) surface groupings of moieties such as amino acids or polysaccharide side chains and may have specific three-dimensional structural characteristics, as well as specific charge characteristics. An epitope may be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule. Antibody “epitope” depends on the methodology used to identify the epitope.

A “leader sequence” (also referred to as “signal peptide” or “signal sequence”) as used herein includes any signal peptide that can be processed by a mammalian cell, including the human B2M leader. Such sequences are well-known in the art.

A “cleavable linker” (also referred to as “protease sequence”) is a peptide substrate cleavable by an enzyme. Operatively, the cleavable linker, upon being cleaved by the enzyme, allows for activation of the present shielded antibody (also referred to as a pro-antibody) with a masking domain, e.g., an IGF2-based masking domain. Preferably, the cleavable linker is selected so that activation occurs at the desired site of action, which can be a site in or near the target cells (e.g., carcinoma cells) or tissues. For example, the cleavable linker is a peptide substrate specific for an enzyme that is specifically or highly expressed in the site of action, such that the cleavage rate of the cleavable linker in the target site is greater than that in sites other than the target site.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The terms also include polypeptides that have co-translational (e.g., signal peptide cleavage) and post-translational modifications of the polypeptide, such as, for example, disulfide-bond formation, glycosylation, acetylation, phosphorylation, proteolytic cleavage, and the like.

Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to PCR amplification or other recombinant DNA methods.

The term “masking domain” (also referred to as “shield,” “shielding domain,” or “mask”) in this disclosure refers to a protein domain that can be fused to an antibody and mask the antibody in binding to its antigen. The shielding domain can mask the antibody from recognizing its target epitope, so the antibody is kept as an inactive shielded antibody form. Upon the removal of the shielding domain, the variable domains of the antibody are exposed and can bind and exert actions to its target.

The term “recombinant,” as used herein to describe a nucleic acid molecule, means a polynucleotide of genomic, cDNA, viral, semisynthetic, and/or synthetic origin, which, by virtue of its origin or manipulation, is not associated with all or a portion of the polynucleotide sequences with which it is associated in nature. The term “recombinant,” as used with respect to a protein or polypeptide, refers to a polypeptide produced by expression from a recombinant polynucleotide. The term “recombinant,” as used with respect to a host cell or a virus, refers to a host cell or virus into which a recombinant polynucleotide has been introduced. Recombinant is also used herein with reference to a material (e.g., a cell, a nucleic acid, a protein, or a vector) that the material has been modified by the introduction of a heterologous material (e.g., a cell, a nucleic acid, a protein, or a vector).

The terms “polynucleotide,” “oligonucleotide,” “nucleic acid” and “nucleic acid molecule” are used interchangeably herein to include a polymeric form of nucleotides, either ribonucleotides or deoxyribonucleotides. This term refers only to the primary structure of the molecule.

“Vector” refers to a polynucleotide capable of being duplicated within a biological system or that can be moved between such systems. Vector polynucleotides typically contain elements, such as origins of replication, polyadenylation signal or selection markers, that function to facilitate the duplication or maintenance of these polynucleotides in a biological system, such as a cell, virus, animal, plant, and reconstituted biological systems utilizing biological components capable of duplicating a vector. The vector polynucleotide may be DNA or RNA molecules, cDNA, or a hybrid of these, single stranded or double stranded.

“Expression vector” refers to a vector that can be utilized in a biological system or in a reconstituted biological system to direct the translation of a polypeptide encoded by a polynucleotide sequence present in the expression vector.

As used herein, the term “heterologous” used in reference to nucleic acid sequences, proteins or polypeptides, means that these molecules are not naturally occurring in the cell from which the heterologous nucleic acid sequence, protein or polypeptide was derived. For example, the nucleic acid sequence coding for a human polypeptide that is inserted into a cell that is not a human cell is a heterologous nucleic acid sequence in that particular context. Whereas heterologous nucleic acids may be derived from different organism or animal species, such nucleic acid need not be derived from separate organism species to be heterologous. For example, in some instances, a synthetic nucleic acid sequence or a polypeptide encoded therefrom may be heterologous to a cell into which it is introduced in that the cell did not previously contain the synthetic nucleic acid. As such, a synthetic nucleic acid sequence or a polypeptide encoded therefrom may be considered heterologous to a human cell, e.g., even if one or more components of the synthetic nucleic acid sequence or a polypeptide encoded therefrom was originally derived from a human cell.

A “host cell,” as used herein, denotes a cell of any type that is capable of being transformed with a nucleic acid or vector of the disclosure so as to produce a polypeptide encoded thereby. For example, the host cell can be an in vivo or in vitro eukaryotic cell or a cell from a multicellular organism (e.g., a cell line) cultured as a unicellular entity, which eukaryotic cells can be, or have been, used as recipients for a nucleic acid (e.g., an expression vector that comprises a nucleotide sequence encoding a multimeric polypeptide of the present disclosure), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an expression vector. For example, a genetically modified eukaryotic host cell is genetically modified by virtue of introduction into a suitable eukaryotic host cell a heterologous nucleic acid, e.g., an exogenous nucleic acid that is foreign to the eukaryotic host cell, or a recombinant nucleic acid that is not normally found in the eukaryotic host cell.

“Specific binding” or “specifically binds” or “binds” refer to an antibody binding to a specific antigen with greater affinity than for other antigens. Typically, the antibody “specifically binds” when the equilibrium dissociation constant (K_{D}) for binding is about 1×10⁻⁸ M or less, for example about 1×10⁻⁹ M or less, about 1×10⁻¹⁰ M or less, about 1×10⁻¹¹ M or less, or about 1×10⁻¹² M or less, typically with the K_{D} that is at least one hundred-fold less than its K_{D} for binding to a non-specific antigen (e.g., BSA, casein). The K_{D} may be measured using standard procedures.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

A “therapeutically effective amount,” “pharmaceutically effective amount,” “effective amount,” or “efficacious amount” refers to the amount of an agent, or combined amounts of two agents, that, when administered to a mammal or other subject for treating a disease, is sufficient to affect such treatment for the disease. The “therapeutically effective amount” will vary depending on the agent(s), the disease and its severity and the age, weight, etc., of the subject to be treated.

Before the present disclosure is further described, it is to be understood that this disclosure is not limited to particular embodiments described, as such may, of course, vary. It is also to be understood that the terminology used herein is for the purpose of describing particular embodiments only and is not intended to be limiting.

### CD38 and Anti-CD38 Antibodies

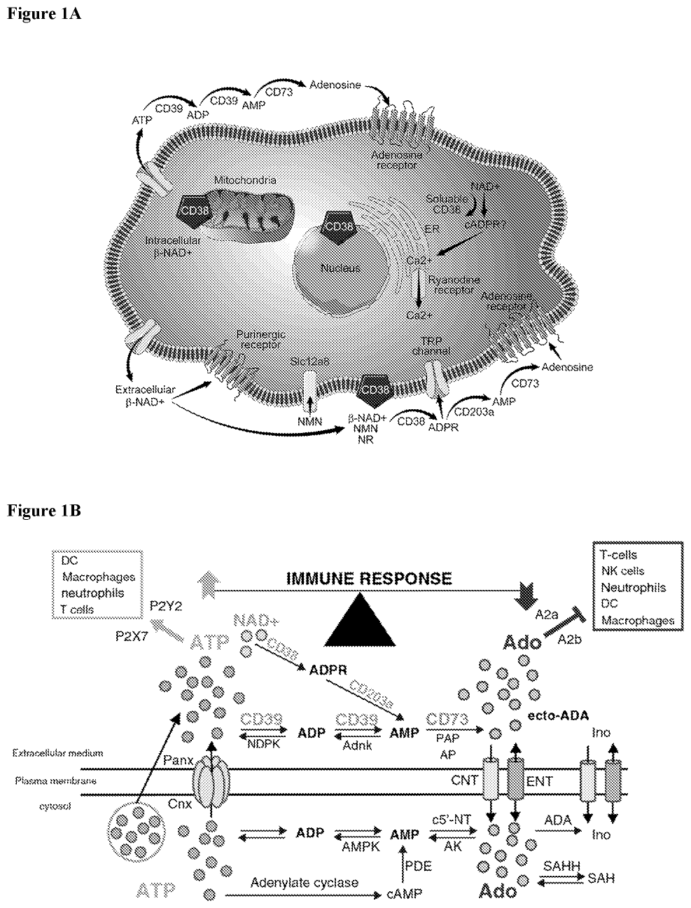

As shown in FIG. 1A, CD38 is predominantly expressed on immune cells and metabolizes nicotinamide nucleotides (NAD⁺ and NMN) to ADPR and cADPR (Hogan, Chini et al. 2019). These metabolites mobilize calcium ions. Although intracellular CD38 is present in the cytoplasm and in the membranes of organelles, a vast majority of CD38 activity is extracellular, which results in degradation of NAD⁺ precursors (e.g., NMN) necessary for NAD⁺ synthesis. As shown in FIG. 1B, extracellular activity of CD38 has wide ranging implications for NAD⁺ homeostasis in the context of infection, metabolic dysfunction, aging, and tumor biology. Multiple immunosuppressive mechanisms impede anti-tumor immunity. Among them, the accumulation of extracellular adenosine is a potent and widespread strategy exploited by tumors to escape immunosurveillance through the activation of purinergic receptors. In the immune system, engagement of A2a and A2b adenosine receptors is a critical regulatory mechanism that protects tissues against excessive immune reactions. In tumors, this pathway is hijacked and hinders anti-tumor immunity, promoting cancer progression (Allard, Beavis et al. 2016).

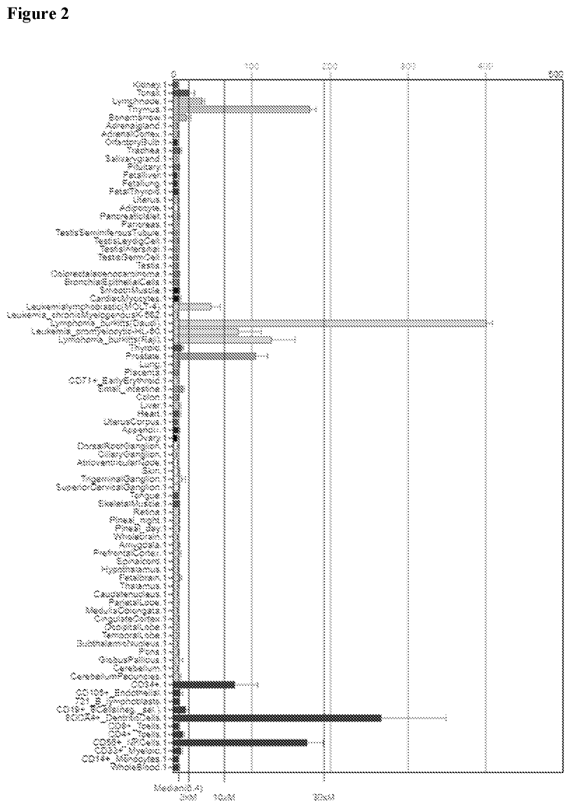

As shown in FIG. 2, CD38 expression is higher in thymus and prostate. However, the expression levels are much higher on activated BDCA4+ dendritic cells, CD56+ NK cells, leukemic cells, and lymphoma cells. This figure is generated using the BioGPS software (Su, Wiltshire et al. 2004, Wu, Jin et al. 2016).

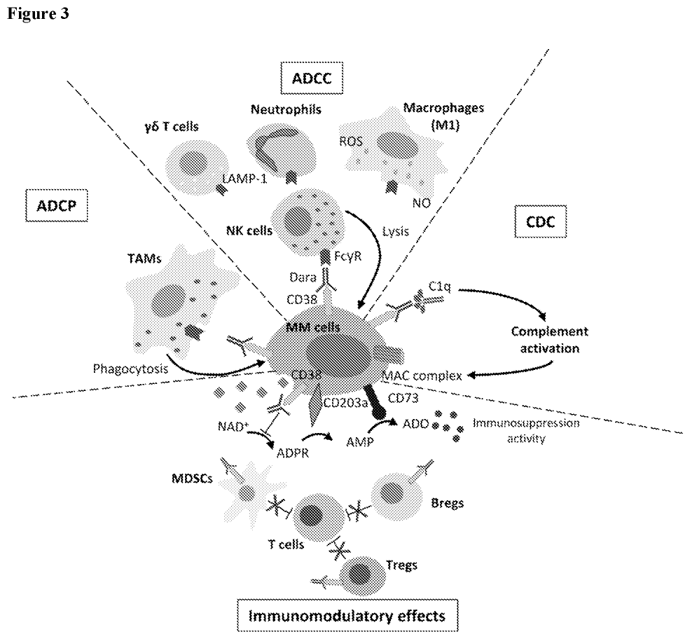

As shown in FIG. 3, anti-CD38 antibodies can recognize CD38 on MM cells to result in anti-MM activity via Fc-dependent mechanisms and via immunomodulatory effects (Saltarella, Desantis et al. 2020). The Fc-dependent mechanisms can comprise (a) antibody-dependent cellular cytotoxicity (ADCC); (b) antibody-dependent cellular phagocytosis (ADCP) via the engagement of the antibody Fc domain to FcγR expressing effector cells (e.g., NK cells, γδ T cells, neutrophils, and macrophages), causing the lysis and/or the phagocytosis of MM cells, respectively; (c) complement-dependent cytotoxicity (CDC) via the engagement of C1q to activate the complement cascade resulting in the assembly of the membrane attack complex (MAC) that can lyse the target cells. Anti-CD38 antibodies can have an immunomodulatory effect by inhibition of CD38 ectoenzymatic activity, resulting in a reduction of immunosuppressive extracellular adenosine (ADO). In addition, there can be elimination of CD38+ immunosuppressive cells (i.e., MDSCs, Tregs, and Bregs) thereby promoting T-cell proliferation and effector functions.

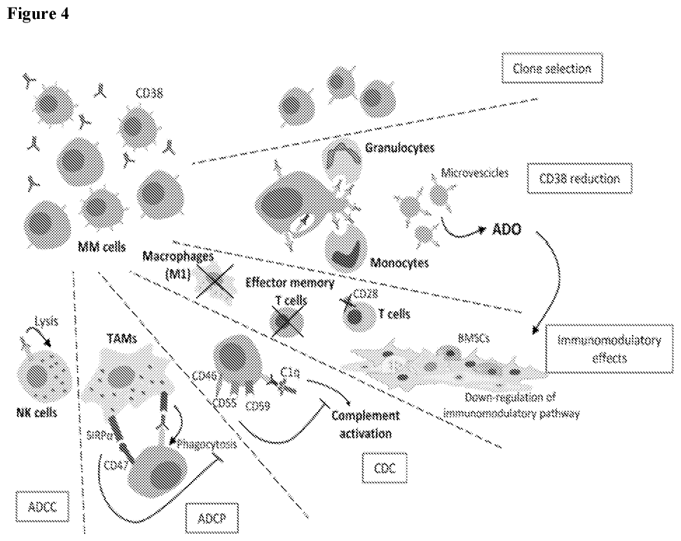

As shown in FIG. 4, anti-CD38 antibodies can induce several mechanisms of resistance including the following: (a) clone selection of CD38dim MM cells thereby minimizing the mAb mechanism on cells; (b) CD38 reduction via CD38 endocytosis, trogocytosis by granulocytes and monocytes, and via release of CD38-expressing microvesicles that contribute to adenosine production and immunosuppression; (c) immunomodulatory effects via downregulation of intracellular pathways in bone marrow-derived stem/stromal cells (BMSCs), a decrease of effector memory T cells, MI macrophages, and of the co-stimulatory CD28 expression on T cells; (d) overexpression of CD46 and of the membrane-associated complement-inhibitory proteins (CD55 and CD59) in MM cells that prevent CDC; (e) MM cell overexpression of CD47 that recognizes immune checkpoint signal regulatory protein α (SIRP) on tumor thereby inhibiting antibody dependent cytotoxic phagocytosis (ADCP); and (f) depletion of CD38+ NK cells via fratricide ADCC.

The primary amino acid sequence of human CD38 is set forth in SEQ NO: 1 in Table 2.

TABLE 2

| SEQ | | |
|---|---|---|
| ID | | |
| NO: | Description | Amino Acid Sequence |
| 1 | Human | MANCEFSPVSGDKPCCRLSRRAQLCLGVSILV |
| | CD38 | LILVVVLAVVVPRWRQQWSGPGTTKRFPETVL |
| | | ARCVKYTEIHPEMRHVDCQSVWDAFKGAFISK |
| | | HPCNITEEDYQPLMKLGTQTVPCNKILLWSRI |
| | | KDLAHQFTQVQRDMFTLEDTLLGYLADDLTWC |
| | | GEFNTSKINYQSCPDWRKDCSNNPVSVFWKTV |
| | | SRRFAEAACDVVHVMLNGSRSKIFDKNSTFGS |
| | | VEVHNLQPEKVQTLEAWVIHGGREDSRDLCQD |
| | | PTIKELESIISKRNIQFSCKNIYRPDKFLQCV |
| | | KNPEDSSCTSEI |

In some embodiments, the present disclosure provides anti-CD38 antibodies and antigen-binding portions thereof. As non-limiting examples, the disclosure provides anti-CD38 heavy and light chain variable region amino acid sequences set forth as SEQ ID NO: 2-30 in Table 3.

In some embodiments, the present disclosure provides an anti-CD38 antibody or an antigen-binding portion thereof, comprising a heavy chain variable region comprising three Complementarity Determining Regions (CDRs), designated as HCDR1, HCDR2, and HCDR3, wherein the HCDR1, HCDR2, and HCDR3 are selected from: SEQ ID NOs: 64, 65, and 66; SEQ ID NOs: 64, 83, and 84; SEQ ID NOs: 64, 65, and 85; and SEQ ID NOs: 64, 65, and 86; respectively.

In some embodiments, the present disclosure provides an anti-CD38 antibody or an antigen-binding portion thereof, comprising a light chain variable region comprising three CDRs, designated as LCDR1, LCDR2, and LCDR3, wherein the LCDR1, LCDR2, and LCDR3 are selected from: SEQ ID NOs: 61, 62, and 63; SEQ ID NOs: 76, 77, and 78; SEQ ID NOs: 79, 80, and 81; and SEQ ID NOs: 82, 62, and 78; respectively.

In some embodiments, the present disclosure provides an anti-CD38 antibody or an antigen-binding portion thereof, comprising:
a heavy chain variable region comprising three Complementarity Determining Regions (CDRs), designated as HCDR1, HCDR2, and HCDR3, wherein the HCDR1, HCDR2, and HCDR3 are selected from: SEQ ID NOs: 64, 65, and 66; SEQ ID NOs: 64, 83, and 84; SEQ ID NOs: 64, 65, and 85; and SEQ ID NOs: 64, 65, and 86; respectively; and a light chain variable region comprising three CDRs, designated as LCDR1, LCDR2, and LCDR3, wherein the LCDR1, LCDR2, and LCDR3 are selected from: SEQ ID NOs: 61, 62, and 63; SEQ ID NOs: 76, 77, and 78; SEQ ID NOs: 79, 80, and 81; and SEQ ID NOs: 82, 62, and 78; respectively

In some embodiments, the disclosure provides an anti-CD38 antibody or antigen binding fragment thereof, comprising a heavy chain sequence comprising an amino acid sequence with at least 85% identity (e.g., 85%, 90%, 95%, 96%, 97%, 98%, 99%, or 100%) to any one of SEQ ID NOs: 12-16 or an antigen-binding portion thereof, and a light chain sequence comprising an amino acid sequence with at least 85% identity (e.g., 85%, 90%, 95%, 96%, 97%, 98%, 99%, or 100%) to any one of SEQ ID NOs: 7-11 or an antigen-binding portion thereof.

In some embodiments, the disclosure provides an anti-CD38 antibody or antigen binding fragment thereof, comprising a light chain sequence and a heavy chain sequence comprising: SEQ ID NOs: 2 and 3; SEQ ID NOs: 7 and 12; SEQ ID NOs: 7 and 13; SEQ ID NOs: 7 and 14; SEQ ID NOs: 7 and 15; SEQ ID NOs: 7 and 16; SEQ ID NOs: 8 and 12; SEQ ID NOs: 8 and 13; SEQ ID NOs: 8 and 14; SEQ ID NOs: 8 and 15; SEQ ID NOs: 8 and 16; SEQ ID NOs: 9 and 12; SEQ ID NOs: 9 and 13; SEQ ID NOs: 9 and 14; SEQ ID NOs: 9 and 15; SEQ ID NOs: 9 and 16; SEQ ID NOs: 10 and 12; SEQ ID NOs: 10 and 13; SEQ ID NOs: 10 and 14; SEQ ID NOs: 10 and 15; SEQ ID NOs: 10 and 16; SEQ ID NOs: 11 and 12; SEQ ID NOs: 11 and 13; SEQ ID NOs: 11 and 14; SEQ ID NOs: 11 and 15; or SEQ ID NOs: 11 and 16; respectively.

In some embodiments, the present disclosure provides an anti-CD38 antibody or an antigen-binding portion thereof, comprising at least one variable-heavy-chain-only single-domain or an antigen-binding portion thereof, wherein the at least one variable-heavy-chain-only single-domain comprises three CDRs (HCDR1, HCDR2, and HCDR3) selected from: SEQ ID NOs: 67, 68, and 69; SEQ ID NOs: 67, 87, and 69; SEQ ID NOs: 67, 88, and 69; SEQ ID NOs: 67, 89, and 69; SEQ ID NOs: 67, 68, and 90; SEQ ID NOs: 70, 91, and 72; SEQ ID NOs: 70, 92, and 72; SEQ ID NOs: 70, 93, and 72; SEQ ID NOs: 94, 95, and 96; SEQ ID NOs: 70, 71, and 97; SEQ ID NOs: 73, 98, and 75; SEQ ID NOs: 73, 99, and 75; SEQ ID NOs: 73, 100, and 75; SEQ ID NOs: 73, 101, and 102; and SEQ ID NOs: 73, 103, and 104; respectively.

In some embodiments, the present disclosure provides an anti-CD38 antibody or an antigen-binding portion thereof, comprising at least one variable-heavy-chain-only single-domain or an antigen-binding portion thereof, wherein the at least one variable-heavy-chain-only (VHO) single-domain comprises an amino acid sequence with at least 85% identity (e.g., 85%, 90%, 95%, 96%, 97%, 98%, 99%, or 100%) to any one of SEQ ID NOs: 4 and 17-30 or an antigen-binding portion thereof.

The anti-CD38 antibodies of the present disclosure encompass full length antibody comprising two heavy chains and two light chains. The antibodies can be human or humanized chimeric antibodies. Humanized antibodies, as used herein, include chimeric antibodies and CDR-grafted antibodies. Chimeric antibodies are antibodies that include a non-human antibody variable region linked to a human constant region. CDR-grafted antibodies are antibodies that include the CDRs from a non-human “donor” antibody linked to the framework region from a human “recipient” antibody. Exemplary human or humanized antibodies include IgG, IgM, IgE, IgA, and IgD antibodies. The present antibodies can be of any class (IgG, IgM, IgE, IgA, IgD, etc.) or isotype. For example, a human antibody can comprise an IgG Fc domain, such as at least one of isotypes, IgG1, IgG2, IgG3, or IgG4.

In some embodiments, the anti-CD38 antibody or antigen-binding portion thereof is selected from the group consisting of a whole antibody, an antibody fragment, a human antibody, humanized antibody, chimeric antibody, a single chain antibody, a conjugate, an antibody mimetic, and a defucosylated antibody. In further examples, the anti-CD38 antibody fragment is selected from the group consisting of a UniBody, a variable heavy only single domain antibody, and a Nanobody. For example, the anti-CD38 antibody fragment is a Nanobody as noted in SEQ ID NO: 17-30. In some examples, the anti-CD38 antibody fragment is selected from the group consisting of a single domain VHH, a single domain VHO, an Affibody, a DARPin, an Anticalin, an Avimer, a Versa body, and a Duocalin.

### Anti-CD3 Antibodies

In some embodiments, the present disclosure provides anti-CD3 antibodies and antigen-binding portions thereof. As non-limiting examples, the disclosure provides for anti-CD3 amino acid sequences set forth as SEQ ID NO: 31-41 in Table 4.

In some embodiments, the present disclosure provides an anti-CD3 antibody or an antigen-binding portion thereof, comprising a heavy chain variable region comprising three Complementarity Determining Regions (CDRs), designated as HCDR1, HCDR2, and HCDR3, wherein the HCDR1, HCDR2, and HCDR3 are selected from: SEQ ID NOs: 105, 108, and 107; SEQ ID NOs: 105, 109, and 107; SEQ ID NOs: 105, 110, and 107; and SEQ ID NOs: 118, 119, and 120; respectively.

In some embodiments, the present disclosure provides an anti-CD3 antibody or an antigen-binding portion thereof, comprising a light chain variable region comprising three CDRs, designated as LCDR1, LCDR2, and LCDR3, wherein the LCDR1, LCDR2, and LCDR3 are selected from: SEQ ID NOs: 114, 112, and 113; and SEQ ID NOs: 115, 116, and 117; respectively.

In some embodiments, the present disclosure provides an anti-CD3 antibody or an antigen-binding portion thereof, comprising:
a heavy chain variable region comprising three Complementarity Determining Regions (CDRs), designated as HCDR1, HCDR2, and HCDR3, wherein the HCDR1, HCDR2, and HCDR3 are selected from: SEQ ID NOs: 105, 108, and 107; SEQ ID NOs: 105, 109, and 107; SEQ ID NOs: 105, 110, and 107; and SEQ ID NOs: 118, 119, and 120;
respectively; and a light chain variable region comprising three CDRs, designated as LCDR1, LCDR2, and LCDR3, wherein the LCDR1, LCDR2, and LCDR3 are selected from: SEQ ID NOs: 114, 112, and 113; and SEQ ID NOs: 115, 116, and 117; respectively.

In some embodiments, the disclosure provides an anti-CD3 antibody or antigen binding fragment thereof comprising a heavy chain sequence comprising an amino acid sequence with at least 85% identity (e.g., 85%, 90%, 95%, 96%, 97%, 98%, 99%, or 100%) to any one of SEQ ID NOs: 32-34 and 39 or an antigen-binding portion thereof, and a light chain sequence comprising an amino acid sequence with at least 85% identity to any one of SEQ ID NOs: 36-38 or an antigen-binding portion thereof.

In some embodiments, the disclosure provides an anti-CD3 antibody or antigen binding fragment thereof, comprising a light chain sequence and a heavy chain sequence comprising: SEQ ID NO: 39 and 38; SEQ ID NO: 41 and 40; SEQ ID NO: 31 and 35; SEQ ID NO: 31 and 36; SEQ ID NO: 31 and 37; SEQ ID NO: 32 and 35; SEQ ID NO: 32 and 36; SEQ ID NO: 32 and 37; SEQ ID NO: 33 and 35; SEQ ID NO: 33 and 36; SEQ ID NO: 33 and 37; SEQ ID NO: 34 and 35; SEQ ID NO: 34 and 36; or SEQ ID NO: 34 and 37; respectively.

The anti-CD3 antibodies of the present disclosure encompass full length antibody comprising two heavy chains and two light chains. The antibodies can be human or humanized antibodies.

In some embodiments, the anti-CD3 antibody or antigen-binding portion thereof is selected from the group consisting of a whole antibody, an antibody fragment, a human antibody, humanized antibody, a single chain antibody, a conjugate, an antibody mimetic, and a defucosylated antibody. In further examples, the anti-CD3 antibody fragment is selected from the group consisting of a UniBody, a variable heavy only single domain antibody, and a Nanobody. In some examples, the anti-CD3 antibody fragment is selected from the group consisting of a single domain VHH, a single domain VHO, an Affibody, a DARPin, an Anticalin, an Avimer, a Versa body, and a Duocalin.

### Bispecific CD3×CD38 Antibodies

In some embodiments, the present disclosure provides a CD3×CD38 bispecific antibody that targets simultaneously proteins linked to CD38-associated pathways as well as proteins that can activate the CD3 T cell activity. In some embodiments, the present disclosure provides a CD3×CD38 bispecific antibody comprising shielding domains. In some embodiments, the present disclosure provides a CD3×CD38 bispecific antibody without shielding domains. CD3 and CD38 targets have differential expression levels in pathological sites and normal tissues. A shielded CD3×CD38 bispecific antibody remains inactive in normal tissues due to the inhibitory effects of the masking domains on the binding domains. The masking domains are cleaved off by proteases in disease sites and the shielded CD3×CD38 bispecific antibody is converted to an active CD3×CD38 bispecific antibody.

In some embodiments, the present disclosure provides a bispecific antibody that targets and binds to human CD38 and CD3 simultaneously, has high affinity, and is capable of effectively blocking CD38 proteins at the protein level. The bispecific antibody binds both CD3 and CD38 proteins and binds to one protein without affecting the binding of the other protein, that is, having the ability to bind CD3 and CD38 simultaneously. The bispecific antibody disclosed herein fills the gap that there is no antibody that simultaneously targets CD3 and CD38. The bispecific antibody disclosed herein inhibits CD38-mediated disease or disorders, such as the proliferation of vascular endothelial cells, human lung cancer cells, human breast cancer cells, human pancreatic cancer cells, and/or human gastric cancer cells.

In some embodiments, the present disclosure provides a bispecific antibody comprising:
a first binding arm comprising:
a first heavy chain fusion protein comprising, from the N- to the C-terminus, a shield A, a protease sequence A, and an IgG heavy chain or an antigen-binding portion thereof, and
a first light chain fusion protein comprising, from the N- to the C-terminus, a shield B, a protease sequence B, and an IgG light chain or an antigen-binding portion thereof,
wherein the IgG heavy chain or an antigen-binding portion thereof and the IgG light chain or an antigen-binding portion thereof of the first binding arm can target a CD3 associated pathway; and
a second binding arm comprising:
a second heavy chain fusion protein comprising, from the N- to the C-terminus, a shield C, a protease sequence C, and an IgG heavy chain or an antigen-binding portion thereof; and
a second light chain fusion protein comprising, from the N- to the C-terminus, a shield D, a protease sequence D, and an IgG light chain or an antigen-binding portion thereof,
wherein the IgG heavy chain or an antigen-binding portion thereof and the IgG light chain or an antigen-binding portion thereof of the second binding arm can target a CD38 associated pathway.
   The shields A-D can be the same or different from one another, and the protease sequences A-D can be the same or different from one another.

In some embodiments, the present disclosure provides a bispecific antibody comprising:
a first binding arm comprises:
a first heavy chain fusion protein comprising, from the N- to the C-terminus, a signal sequence A-a shield A-a linker A-a protease sequence A-a linker B-an IgG heavy chain or an antigen-binding portion thereof, and
a first light chain fusion protein comprising, from the N- to the C-terminus, a signal sequence B-a shield B-a linker C-a protease sequence B-a linker D-an IgG light chain or an antigen-binding portion thereof,
wherein the IgG heavy chain or an antigen-binding portion thereof and the IgG light chain or an antigen-binding portion thereof of the first binding arm are capable of targeting a CD3 associated pathway; and
the second binding arm comprises:
a second heavy chain fusion protein comprising, from the N- to the C-terminus, a signal sequence C-a shield C-a linker E-a protease sequence C-a linker F-an IgG heavy chain or an antigen-binding portion thereof; and
a second light chain fusion protein comprising, from the N- to the C-terminus, a signal sequence D-a shield D-a linker G-a protease sequence D-a linker H-an IgG light chain or an antigen-binding portion thereof,
wherein the IgG heavy chain or an antigen-binding portion thereof and the IgG light chain or an antigen-binding portion thereof of the second binding arm are capable of targeting a CD38 associated pathway.
   The signal sequences A-D can be the same or different from one another, and the linkers A-H can be the same or different from one another.

In some embodiments, the present disclosure provides a bispecific antibody comprising:
a first binding arm comprising:
   a first heavy chain fusion protein comprising, from the N- to the C-terminus, a shield A, a protease sequence A, and an IgG heavy chain or an antigen-binding portion thereof, and
a first light chain fusion protein comprising, from the N- to the C-terminus, a shield B, a protease sequence B, and an IgG light chain or an antigen-binding portion thereof,
wherein the IgG heavy chain or an antigen-binding portion thereof and the IgG light chain or an antigen-binding portion thereof of the first binding arm are capable of targeting a CD3 associated pathway; and
a second binding arm comprising:
a second heavy chain fusion protein comprising, from the N- to the C-terminus, a shield C, a protease sequence C, and an IgG heavy chain comprising at least one variable-heavy-chain-only (VHO) single domain or an antigen-binding portion thereof,
wherein the at least one variable-heavy-chain-only single domain or an antigen-binding portion thereof is capable of targeting a CD38 associated pathway.
   The shields A-C can be the same or different from one another, and the protease sequences A-C can be the same or different from one another.

In some embodiments, the present disclosure provides a bispecific antibody comprising:
a first binding arm comprises:
a first heavy chain fusion protein comprising, from the N- to the C-terminus, a signal sequence A-a shield A-a linker A-a protease sequence A-a linker B-an IgG heavy chain or an antigen-binding portion thereof, and
a first light chain fusion protein comprising, from the N- to the C-terminus, a signal sequence B-a shield B-a linker C-a protease sequence B-a linker D-an IgG light chain or an antigen-binding portion thereof,
wherein the IgG heavy chain or an antigen-binding portion thereof and the IgG light chain or an antigen-binding portion thereof of the first binding arm are capable of targeting a CD3 associated pathway; and
the second binding arm comprises:
a second heavy chain fusion protein comprising, from the N- to the C-terminus, a signal sequence C-a shield C-a linker E-a protease sequence C-a linker F-an IgG heavy chain comprising at least one variable-heavy-chain-only single-domain or an antigen-binding portion thereof,
wherein at least one variable-heavy-chain-only single-domain is capable of targeting a CD38 associated pathway.
   The signal sequences A-C can be the same or different from one another, and the linkers A-F can be the same or different from one another.

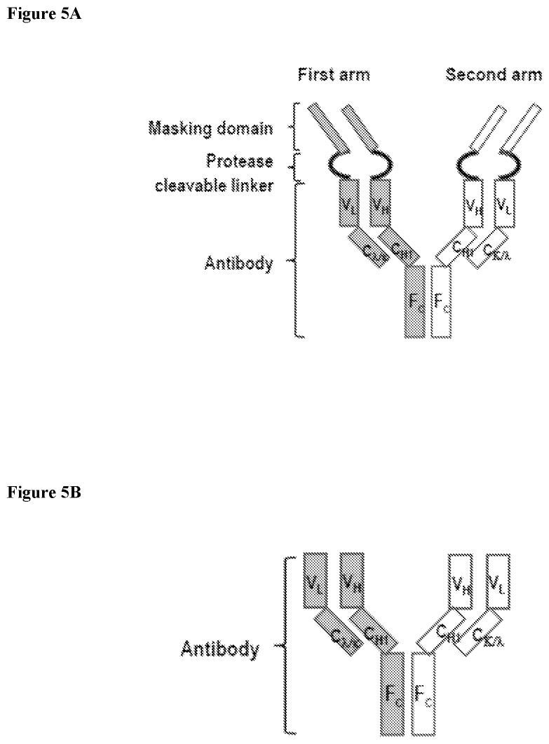

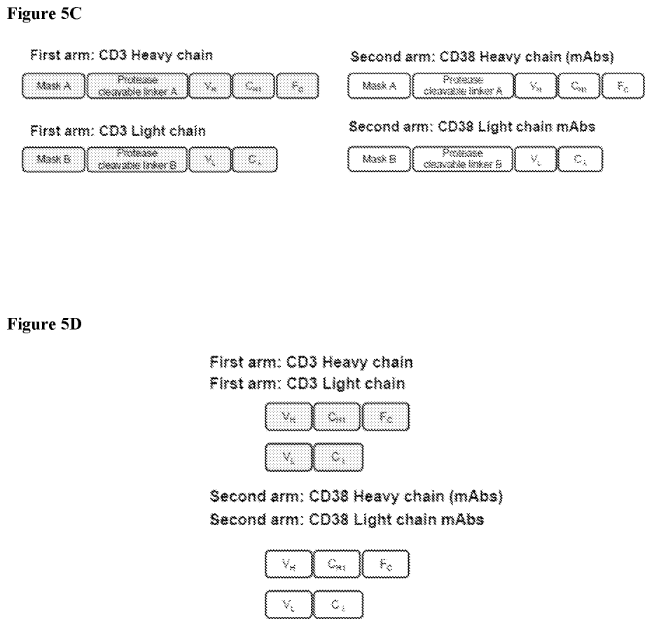

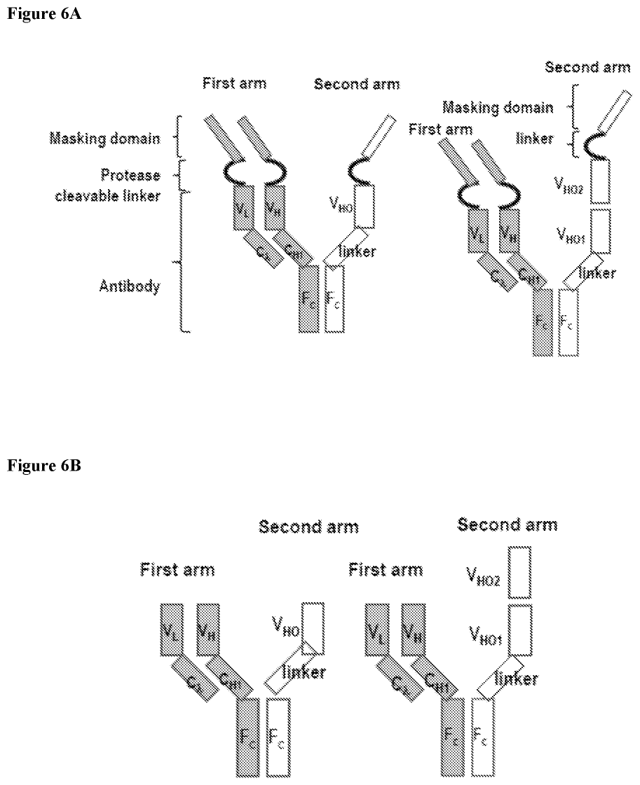

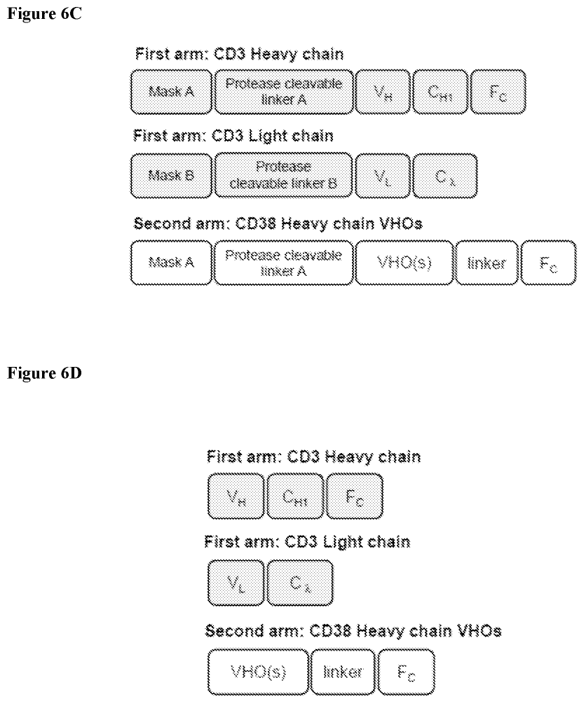

In some embodiments, the bispecific antibody consists of two sets of light chain fusions and two sets of heavy chain fusions. For example, the structures of the light chain fusions and the heavy chain fusions from the respective parental antibodies are shown in FIG. 5. For example, a bispecific antibody may comprise a human IgG1 heavy chain fusion comprising amino acid sequences from N- to the C-terminus: a signal sequence A-shield A-linker A-protease sequence A-linker B-IgG1 heavy chain; and a human IgG1 light chain fusion comprising amino acid sequences from the N- to the C-terminus, a signal sequence B-shield B-linker B-protease sequence B-linker C-IgG1 light chain. Signal sequence A can be the same or different from Signal sequence B. Shield A can be the same or different from shield B. Linker A can be the same or different from linker B. Protease sequence B can be same or different from protease sequence A. In some embodiments, the bispecific antibody consists of two sets of heavy chain fusions and one set of light chain fusion as illustrated in FIGS. 6A and 6B.

The present disclosure provides a bispecific antibody that can be generated using well established point mutations in the CH1, CH2, and CH3 domains via controlled Fab arm exchange or via co-expression. In some embodiment, all constructs are symmetric so that there is no preference for the selection of point mutations of the respective parental antibodies.

FIGS. 5-7 illustrate several formats of a bispecific antibody disclosed herein, with or without masking domains. FIG. 5A shows a bispecific antibody that has two different sets of heavy chain (HC) and light chain (LC) pairing as indicated under the notations of “first arm” (targeting CD3) and “second arm” (targeting CD38). FIG. 5B shows that the CD3 arm (first arm) of the bispecific Ab can comprise a human IgG with an HC and an LC, wherein the light chain fusion comprises from the N-terminus to the C-terminus: Mask domain B, protease cleavable linker B, LC variable region VL, and constant light chain CL (e.g., Cλ), and wherein the heavy chain fusion comprises from the N-terminus to the C-terminus: Mask domain A, protease cleavable linker A, an HC variable region VH, a CH1 domain, and an Fc region. FIG. 5C shows that the CD38 binding arm (second arm) of the bispecific Ab can comprise a human IgG with an HC and an LC, wherein the light chain fusion comprises from the N-terminus to the C-terminus: Mask domain B, protease cleavable linker B, LC variable region VL, and constant light chain CL (e.g., Cκ or C_{λ}), and wherein the heavy chain comprises from the N-terminus to the C-terminus: Mask domain A, protease cleavable linker A, an HC variable region VH or VHO in tandem, a CH1 domain, and an Fc region. The Fc region may further be genetically fused to a homing domain (HD). Different or same linkers can be placed between the Mask A and protease cleavable linker A, protease cleavable linker A and VH, and Fc domain and HD domain. Different or same linkers can be put between the Mask B and protease cleavable linker B, protease cleavable linker B and VH or VHO in tandem, and Fc domain and HD domain. The heavy chain and light chain can have the same or different complementary Mask domains, and protease cleavable linkers.

FIG. 6A shows that a bispecific antibody comprises a single VHO in the “second arm,” an arm targeting CD38, and FIG. 6B shows that the CD38 binding arm (second arm) comprises two VHOs fused together. FIG. 6C shows that the CD3 arm (first arm) of the bispecific Ab illustrated in FIGS. 6A and 6B can comprise a human IgG having an HC and an LC as illustrated in FIG. 5B. FIG. 6C also shows that the CD38 binding arm comprises from the N-terminus to the C-terminus: a Mask domain A, a protease cleavable linker A, an HC VHO variable region, a CH1 domain, and an Fc region. More than one VHO fusion separated by linker domains can be present for higher specificity. The CD38 binding arm can have different linkers and masking domains than the CD3 binding arm.

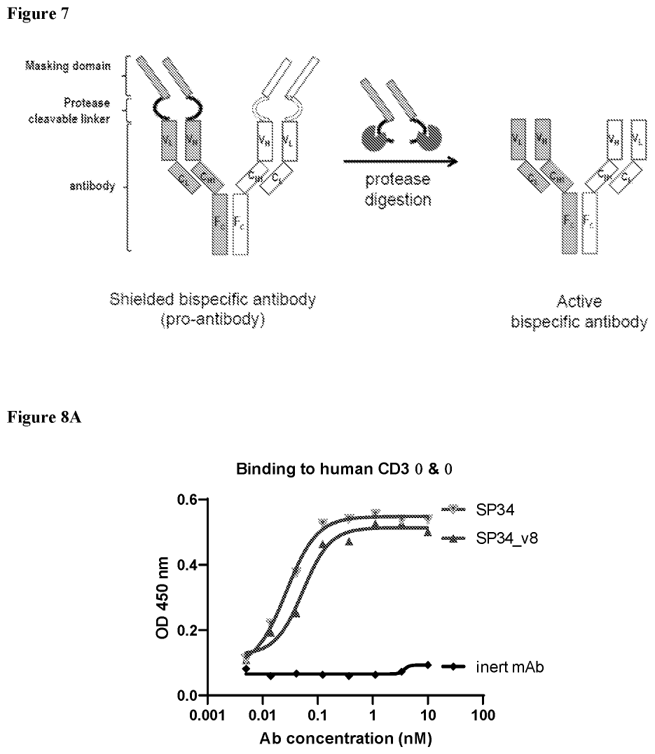

FIG. 7 is a schematic representation showing the removal of the masking domains from a CD38×CD3 bispecific antibody. Proteases that are in higher concentrations in the tumor microenvironment can cut along the protease cleavable linkers to convert a shielded bispecific antibody to an active bispecific antibody.

Genetic fusion of a VHO domain in a bispecific antibody disclosed herein, e.g., comprising a human VHO single domain linked to either a CH1-hinge-CH2-CH3 domain of human IgG1 or a hinge-CH2-CH3 domain of human IgG1, yields a highly soluble chimeric heavy chain antibody at half the size of a conventional antibody (75 vs. 150 kDa) as shown in FIGS. 5 and 6. Such a construct may penetrate tissues better than conventional antibodies. In addition, a bispecific molecule disclosed herein would take advantage of its added tumor cell growth inhibition efficacy in diseased tissues yet mitigating the increased toxicity concern by shielding the binding epitopes of the CD3 and CD38 in normal tissues as shown in FIG. 7. Therefore, a bispecific antibody disclosed herein has effective targeting of CD38 for oncology and other disorders rooted from CD38 over-expression, but with reduced safety concerns, to extend the role of CD38 in the context of therapy of many cancers as well as other non-oncology indications.

In some embodiments of the bispecific antibody disclosed herein, the binding arm that targets CD3 has a binding valency of one. The bispecific antibody is preferably designed to have monovalent CD3-binding (i.e., one Fab arm binding to the epitope of CD3). Bivalent CD3 binding is linked to excess of activation-induced cell death in effector cells that would limit the efficacy of T cell redirection. In addition, a bivalent CD3 redirection molecule can result in tumor antigen-independent immune effector cell activation that can increase systemic toxicity in the patient. The use of a high affinity of anti-CD3 antibody Fab arm can also increase toxicity. High affinity variants of anti-CD3 Fab or scFv are typically poorly tolerated in cynomolgus monkeys because of resulting extensive cytokine release. High affinity for CD3 also shifted bispecific antibody biodistribution from tumors to CD3 rich tissue that led to increased risk of cytokine release syndrome. Thus, it is highly desirable for the binding affinity of the anti-CD3 arm to be lower than that of anti-CD38 arm. Bivalent anti-CD3 agents can increase avidity in binding to CD3 and could lead to increase in cytokine release syndrome from off-tumor CD3 positive cells.

In some embodiments of the bispecific antibody disclosed herein, the second binding arm targeting a CD38 associated pathway, such as CD38, may comprise, in tandem, two to four, such as two or three, IgG variable-heavy-chain-only single domains or antigen-binding portion thereof, wherein the two or three IgG variable heavy chain only single domains or an antigen-binding portion thereof are optionally connected via one or more linkers. The second binding arm targeting a CD38 associated pathway, such as CD38, can be monovalent, bivalent, trivalent, tetravalent, etc. For example, there can be a dual or tri or quad epitope recognition for CD38 in a bispecific antibody to enhance specificity of engagement.

Long term administration of anti-CD3 or anti-CD38 biologics drugs pose a great risk factor for patients. In some embodiments, the present disclosure provides a combination of shields that can form intermolecular interactions to block Fab arm engagement to their respective epitopes. The shields can be fused to the heavy chain and/or the light chain domains. The shields can block the Fab CDR regions from binding to the antigen via steric hindrance. The presence of shields can minimize systemic toxicity of a CD3×CD38 bispecific antibody disclosed herein, and may increase the safety profile and therapeutic window of the respective arms in the bispecific antibody. The shields can be removed by a protease and/or other in situ specific enzymes that are found in the tumor microenvironment. The shielding domains (also referred to as shields, masks, or masking domains) in a bispecific antibody disclosed herein, e.g., shields A, B, C, D, E, and F, can be the same or different. In some embodiments, the shield A, shield B, shield C, shield D, shield E, and shield F are each independently selected from the amino acid sequences set forth in SEQ ID NOs: 42-52.

The protease sequences in a bispecific antibody disclosed herein, e.g., protease sequence A, B, C, D, E, and F, can be the same or different. In some embodiments, the protease sequence A, protease sequence B, protease sequence C, protease sequence D, protease sequence E, and protease sequence F are each independently selected from amino acid sequence set forth in SEQ ID NOs: 53-60.

The peptide linkers in a bispecific antibody disclosed herein, e.g., linker A, B, C, D, E, and F, can be the same or different.

In some embodiments, a bispecific antibody disclosed herein comprises a heavy chain sequence selected from SEQ ID NOs: 3 and 12-16 and a light chain sequence selected from SEQ ID NOs: 2 and 7-11.

In some embodiments, a bispecific antibody disclosed herein may comprise multiple binding arms tarting CD38. For example, a bispecific antibody disclosed herein comprises a first CD38 binding arm comprising light chain comprising SEQ ID NO: 2 and a heavy chain comprising SEQ ID NO: 3; a second CD38 binding arm comprising a light chain sequence selected from SEQ ID NOs: 7-11 and a heavy chain sequence selected from SEQ ID NOs: 12-16.

In some embodiments, a bispecific antibody disclosed herein comprises a binding arm that can target CD38, wherein the binding arm comprises a human IgG1 heavy chain and a light chain sequence selected from SEQ ID NOs: 2-6.

### Leader Sequences

In certain embodiments, a leader peptide is incorporated to drive the secretion of an antibody, e.g., a shielded CD3×CD38 bispecific antibody, described herein into the cell culture supernatant as a secreted respective parental antibody protein. Any leader peptide for any known secreted proteins/peptides can be used.

As used herein, a “leader peptide,” “lead peptide,” or “signal peptide” includes a short peptide, usually 16-30 amino acids in length, that is present at the N-terminus of most of newly synthesized proteins that are destined towards the secretory pathway. Although lead peptides are extremely heterogeneous in sequence, and many prokaryotic and eukaryotic lead peptides are functionally interchangeable even between different species, the efficiency of protein secretion may be strongly determined by the sequence of the lead/signal peptide.

In certain embodiments, the leader peptide is from a protein residing either inside certain organelles (such as the endoplasmic reticulum, Golgi, or endosomes), secreted from the cell, or inserted into most cellular membranes.

In certain embodiments, the leader peptide is from a eukaryotic protein.

In certain embodiments, the leader peptide is from a secreted protein, e.g., a protein secreted outside a cell.

In certain embodiments, the leader peptide is from a transmembrane protein.

In certain embodiments, the leader peptide contains a stretch of amino acids that is recognized and cleaved by a signal peptidase.

In certain embodiments, the leader peptide does not contain a cleavage recognition sequence of a signal peptidase.

In certain embodiments, the leader peptide is a signal peptide for tissue plasminogen activator (tPA), herpes simplex virus glycoprotein D (HSV gD), a growth hormone, a cytokine, a lipoprotein export signal, CD2, CD3δ, CD3ε, CD3γ, CD3ζ, CD4, CD8α, CD19, CD28, 4-1BB or GM-CSFR, or S. cerevisiae mating factor α-1 signal peptide.

In some embodiments, a leader sequence as described herein may be a mammalian CD4 or CD8 leader sequence, including but not limited to, e.g., a human CD4 or CD8 leader sequence, a non-human primate CD4 or CD8 leader sequence, a rodent CD4 or CD8 leader sequence, and the like. In some embodiments, a CD4 or CD8 leader comprises an amino acid sequence having at least 75%, at least 80%, at least 85%, at least 90%, at least 95%, at least 98%, at least 99%, or 100%, amino acid sequence identity with the human CD4 or CD8 leader sequences.

### Anti-CD3 and Anti-CD38 Antibodies and Fragments Applicable for Shielded CD3×CD38 Bispecific Antibody Design

In some embodiments, the present disclosure provides anti-CD3 and anti-CD38 antibodies and antigen-binding fragments thereof for CD3×CD38 bispecific antibody designs. The fabs of the CD3 and CD38 antibodies or antigen-binding fragments thereof can be attached to shields via protease-cleavage linker sequences to make shielded CD3×CD38 bispecific antibodies as disclosed herein.

The anti-CD3 and anti-CD38 antibodies and fragments applicable for a shielded CD3×CD38 bispecific antibody design of the present disclosure encompass full length antibody comprising two heavy chains and two light chains. The antibodies can be human or humanized antibodies.

In some embodiments, the anti-CD38 antibody or anti-CD3 antibody is selected from the group consisting of a whole antibody, an antibody fragment, a human or humanized antibody, a single chain antibody, a conjugate, an antibody mimetic, and a defucosylated antibody. In further examples, the anti-CD38 or anti-CD3 antibody fragment is selected from the group consisting of a UniBody, a variable heavy only single domain antibody, and a Nanobody. For example, the anti-CD38 antibody fragment is a Nanobody as noted in SEQ ID NOs: 17-30. In some examples, the anti-CD38 or anti-CD3 antibody is selected from the group consisting of a single domain VHH, a single domain VHO, an Affibody, a DARPin, an Anticalin, an Avimer, a Versa body, and a Duocalin. As non-limiting examples, the disclosure provides the anti-CD38 heavy and light chain variable region amino acid sequences set forth as SEQ ID NOs: 2-30 in Table 3. As non-limiting examples, the disclosure provides for CD3 construct amino acid sequences set forth as SEQ ID NOs: 31-41 in Table 4. A bispecific antibody disclosed herein can be made comprising any suitable combination of the amino acid sequences set forth in Tables 3 and 4.

TABLE 3

| Description | Amino Acid Sequence | CDRs |
|---|---|---|
| CD38 LC1 | DIVMTQSHLSMSTSLGDPVSITCKASQDVST | CDR1: |
| | VVAWYQQKPGQSPRRLIYSASYRYIGVPDR | KASQDVSTVVA |
| | FTGSGAGTDFTFTISSVQAEDLAVYYCQQH | (SEQ ID NO: 61) |
| | YSPPYTFGGGTKLEIK (SEQ ID NO: 2) | CDR2: |
| | | SASYRYI |
| | | (SEQ ID NO: 62) |
| | | CDR3: |
| | | QQHYSPPYT |
| | | (SEQ ID NO: 63) |
| CD38 HC1 | QVQLVQSGAEVAKPGTSVKLSCKASGYTFT | CDR1: |
| | DYWMQWVKQRPGQGLEWIGTIYPGDGDT | DYWMQ |
| | GYAQKFQGKATLTADKSSKTVYMHLSSLA | (SEQ ID NO: 64) |
| | SEDSAVYYCARGDYYGSNSLDYWGQGTSV | CDR2: |
| | TVSS (SEQ ID NO: 3) | TIYPGDGDTGYA |
| | | QKFQG |
| | | (SEQ ID NO: 65) |
| | | CDR3: |
| | | GDYYGSNSLDY |
| | | (SEQ ID NO: 66) |
| CD38 5FIO | EVQLQESGGGLVQAGHSLRLSCVGSGSRFD | CDR1: |
| VHO1 | NYAMGWFRQAPGKEREFVAAISWSSGTTR | NYAMG |
| | YLDTVKGRFTISRDNAKSTVYLQMNSLKPE | (SEQ ID NO: 67) |
| | DTAVYYCAARYQPRYYDSGDMDGYEYDN | CDR2: |
| | WGQGTQVTVSS (SEQ ID NO: 4) | AISWSSGTTRYLD |
| | | TVKG |
| | | (SEQ ID NO: 68) |
| | | CDR3: |
| | | RYQPRYYDSGDM |
| | | DGYEYDN |
| | | (SEQ ID NO: 69) |
| CD38 5F1K | EVQLQESGGGLVQAGGSLRLSCTGSGRTFR | CDR1: |
| VHO1 | NYPMAWFRQAPGKEREFVAGITWVGASTL | NYPMA |
| | YADFAKGRFTISRDNAKNTVYLQMNSLKPE | (SEQ ID NO: 70) |
| | DTAVYSCAAGRGIVAGRIPAEYADWGQGT | CDR2: |
| | QVTVSS (SEQ ID NO: 5) | GITWVGASTLYA |
| | | DFAKG |
| | | (SEQ ID NO: 71) |
| | | CDR3: |
| | | GRGIVAGRIPAEY |
| | | AD |
| | | (SEQ ID NO: 72) |
| CD38 5F21 | EVQLQESGGGSVQAGGSLTLSCTASGLLFRL | CDR1: |
| VHO1 | ASMGWYRQAPGKERELIATITVGGKTNYK | LASMG |
| | DSVQGRFIITRDNTGDNTKSTVTLQMNRLKP | (SEQ ID NO: 73) |
| | EDTAVYYCNTASPAVGADTWGQGTRVTVS | CDR2: |
| | S (SEQ ID NO: 6) | TITVGGKTNYKD |
| | | SVQG |
| | | (SEQ ID NO: 74) |
| | | CDR3: |
| | | ASPAVGADT |
| | | (SEQ ID NO: 75) |
| CD38 LC2 | DIQMTQSPSSLSASVGDRVTITCKASQDVST | CDR1: |
| | VVAWYQQKPGKAPKRLIYSASYRYIGVPSR | KASQDVSTVVA |
| | FSGSGSGTDFTFTISSLQPEDIATYYCQQHYS | (SEQ ID NO: 61) |
| | PPYTFGQGTKLEIK (SEQ ID NO: 7) | CDR2: |
| | | SASYRYI |
| | | (SEQ ID NO: 62) |
| | | CDR3: |
| | | QQHYSPPYT |
| | | (SEQ ID NO: 63) |
| CD38 LC3 | DVVMTQSPLSLPVTLGQPASISCKASQDVST | CDR1: |
| | VVAWYQQRPGQSPRRLIYSASYRYIGVPDR | KASQDVSTVVA |
| | FSGSGSGTDFTLKISRVEAEDVGVYYCQQH | (SEQ ID NO: 61) |
| | YSPPYTFGQGTKLEIK (SEQ ID NO: 8) | CDR2: |
| | | SASYRYI |
| | | (SEQ ID NO: 62) |
| | | CDR3: |
| | | QQHYSPPYT |
| | | (SEQ ID NO: 63) |
| CD38 LC4 | DIVMTQSHLSMSTSLGDPVSITCKASQDVHT | CDR1: |
| | EVAWYQQKPGQSPRRLIYRASYRYPGVPDR | KASQDVHTEVA |
| | FTGSGAGTDFTFTISSVQAEDLAVYYCQQH | (SEQ ID NO: 76) |
| | YLPPYTFGGGTKLEIK (SEQ ID NO: 9) | CDR2: |
| | | RASYRYP |
| | | (SEQ ID NO: 77) |
| | | CDR3: |
| | | QQHYLPPYT |
| | | (SEQ ID NO: 78) |
| CD38 LC5 | DIVMTQSHLSMSTSLGDPVSITCKASQDVFT | CDR1: |
| | NVAWYQQKPGQSPRRLIYFASYRYFGVPDR | KASQDVFTNVA |
| | FTGSGAGTDFTFTISSVQAEDLAVYYCQQH | (SEQ ID NO: 79) |
| | YEPPYTFGGGTKLEIK (SEQ ID NO: 10) | CDR2: |
| | | FASYRYF |
| | | (SEQ ID NO: 80) |
| | | CDR3: |
| | | QQHYEPPYT |
| | | (SEQ ID NO: 81) |
| CD38 LC6 | DIVMTQSHLSMSTSLGDPVSITCKASQDVYT | CDR1: |
| | VVAWYQQKPGQSPRRLIYSASYRYIGVPDR | KASQDVYTVVA |
| | FTGSGAGTDFTFTISSVQAEDLAVYYCQQH | (SEQ ID NO: 82) |
| | YLPPYTFGGGTKLEIK (SEQ ID NO: 11) | CDR2: |
| | | SASYRYI |
| | | (SEQ ID NO: 62) |
| | | CDR3: |
| | | QQHYLPPYT |
| | | (SEQ ID NO: 78) |
| CD38 HC2 | QVQLVQSGAEVKKPGSSVKVSCKASGYTFT | CDR1: |
| | DYWMQWVRQAPGQGLEWIGTIYPGDGDT | DYWMQ |
| | GYAQKFQGRVTITADESTSTAYMELSSLRS | (SEQ ID NO: 64) |
| | EDTAVYYCARGDYYGSNSLDYWGQGTTVT | CDR2: |
| | VSS (SEQ ID NO: 12) | TIYPGDGDTGYA |
| | | QKFQG |
| | | (SEQ ID NO: 65) |
| | | CDR3: |
| | | GDYYGSNSLDY |
| | | (SEQ ID NO: 66) |
| CD38 HC3 | QVQLVQSGAEVKKPGASVKVSCKASGYTFT | CDR1: |
| | DYWMQWVRQAPGQGLEWIGTIYPGDGDT | DYWMQ |
| | GYAQKFQGRATLTADTSTSTVYMELSSLRS | (SEQ ID NO: 64) |
| | EDTAVYYCARGDYYGSNSLDYWGQGTTVT | CDR2: |
| | VSS (SEQ ID NO: 13) | TIYPGDGDTGYA |
| | | QKFQG |
| | | (SEQ ID NO: 65) |
| | | CDR3: |
| | | GDYYGSNSLDY |
| | | (SEQ ID NO: 66) |
| CD38 HC4 | QVQLVQSGAEVAKPGTSVKLSCKASGYHFR | CDR1: |
| | DYWMQWVKQRPGQGLEWIGTIYPGYGDT | DYWMQ |
| | GYAQKFQGKATLTADKSSKTVYMHLSSLA | (SEQ ID NO: 64) |
| | SEDSAVYYCARGDYYGTNSLDYWGQGTSV | CDR2: |
| | TVSS (SEQ ID NO: 14) | TIYPGYGDTGYA |
| | | QKFQG |
| | | (SEQ ID NO: 83) |
| | | CDR3: |
| | | GDYYGTNSLDY |
| | | (SEQ ID NO: 84) |
| CD38 HC5 | QVQLVQSGAEVAKPGTSVKLSCKASGYRFT | CDR1: |
| | DYWMQWVKQRPGQGLEWIGTIYPGDGDT | DYWMQ |
| | GYAQKFQGKATLTADKSSKTVYMHLSSLA | (SEQ ID NO: 64) |
| | SEDSAVYYCARGDYYGQNSLDYWGQGTSV | CDR2: |
| | TVSS (SEQ ID NO: 15) | TIYPGDGDTGYA |
| | | QKFQG |
| | | (SEQ ID NO: 65) |
| | | CDR3: |
| | | GDYYGQNSLDY |
| | | (SEQ ID NO: 85) |
| CD38 HC6 | QVQLVQSGAEVAKPGTSVKLSCKASGYTFT | CDR1: |
| | DYWMQWVKQRPGQGLEWIGTIYPGDGDT | DYWMQ |
| | GYAQKFQGKATLTADKSSKTVYMHLSSLA | (SEQ ID NO: 64) |
| | SEDSAVYYCARGDYYGENSLDYWGQGTSV | CDR2: |
| | TVSS (SEQ ID NO: 16) | TIYPGDGDTGYA |
| | | QKFQG |
| | | (SEQ ID NO: 65) |
| | | CDR3: |
| | | GDYYGENSLDY |
| | | (SEQ ID NO: 86) |
| CD38 5FIO | QVQLVQSGAEVKKPGASVKVSCKASGSRFD | CDR1: |
| VHO2 | NYAMGWFRQAPGQEREFMGAISWSSGTTR | NYAMG |
| | YAQKVQGRVTMTRDTSTSTVYMELSSLRSE | (SEQ ID NO: 67) |
| | DTAVYYCAARYQPRYYDSGDMDGYEYDN | CDR2: |
| | WGQGTLVTVSS (SEQ ID NO: 17) | AISWSSGTTRYAQ |
| | | KVQ |
| | | (SEQ ID NO: 87) |
| | | CDR3: |
| | | RYQPRYYDSGDM |
| | | DGYEYDN |
| | | (SEQ ID NO: 69) |
| CD38 5FIO | QVTLKESGPVLVKPTETLTLTCTGSGSRFDN | CDR1: |
| VHO3 | YAMGWFRQPPGKEREFLAAISWSSGTTRYS | NYAMG |
| | TSLKSRLTISRDTSKSQVVLTMTNMDPVDT | (SEQ ID NO: 67) |
| | ATYYCAARYQPRYYDSGDMDGYEYDNWG | CDR2: |
| | QGTLVTVSS (SEQ ID NO: 18) | AISWSSGTTRYST |
| | | SLKS |
| | | (SEQ ID NO: 88) |
| | | CDR3: |
| | | RYQPRYYDSGDM |
| | | DGYEYDN |
| | | (SEQ ID NO: 69) |
| CD38 5FIO | EVQLVESGGGLVQPGGSLRLSCAASGSRFD | CDR1: |
| VHO4 | NYAMGWFRQAPGKEREFVAAISWSSGTTR | NYAMG |
| | YVDSVKGRFTISRDNAKNSVYLQMNSLRAE | (SEQ ID NO: 67) |
| | DTAVYYCAARYQPRYYDSGDMDGYEYDN | CDR2: |
| | WGQGTLVTVSS (SEQ ID NO: 19) | AISWSSGTTRYVD |
| | | SVKG |
| | | (SEQ ID NO: 89) |
| | | CDR3: |
| | | RYQPRYYDSGDM |
| | | DGYEYDN |
| | | (SEQ ID NO: 69) |
| CD38 5FIO | DVQLQESGGGLVQAGHSLRLSCVGSGDRFD | CDR1: |
| VHO5 | NYAMGWFRQAPGKEREFVAAISWSSGTTR | NYAMG |
| | YLDTVKGRFTISRDNAKSTVYLQMNSLKPE | (SEQ ID NO: 67) |
| | DTAVYYCAARYQNRFYDSGDMDGYEYDN | CDR2: |
| | WGQGTQVTVSS (SEQ ID NO: 20) | AISWSSGTTRYLD |
| | | TVKG |
| | | (SEQ ID NO: 68) |
| | | CDR3: |
| | | RYQNRFYDSGDM |
| | | DGYEYDN |
| | | (SEQ ID NO: 90) |
| CD38 5FIOK | QMQLVQSGAEVKKTGSSVKVSCKGSGRTFR | CDR1: |
| VHO2 | NYPMAWFRQAPGQEREFMGGITWVGASTL | NYPMA |
| | YAQKAQDRVTITRDRSMSTAYMELSSLRSE | (SEQ ID NO: 70) |
| | DTAMYSCAAGRGIVAGRIPAEYADWGQGT | CDR2: |
| | LVTVSS (SEQ ID NO: 21) | GITWVGASTLYA |
| | | QKAQD |
| | | (SEQ ID NO: 91) |
| | | CDR3: |
| | | GRGIVAGRIPAEY |
| | | AD |
| | | (SEQ ID NO: 72) |
| CD38 5FIOK | QVTLKESGPVLVKPTETLTLTCTGSGRTFRN | CDR1: |
| VHO3 | YPMAWFRQPPGKEREFLAGITWVGASTLY | NYPMA |
| | STSAKSRLTISRDTSKSQVVLTMTNMDPVDT | (SEQ ID NO: 70) |
| | ATYSCAAGRGIVAGRIPAEYADWGQGTLV | CDR2: |
| | TVSS (SEQ ID NO: 22) | GITWVGASTLYST |
| | | SAKS |
| | | (SEQ ID NO: 92) |
| | | CDR3: |
| | | GRGIVAGRIPAEY |
| | | AD |
| | | (SEQ ID NO: 72) |
| CD38 5FIOK | QVQLVESGGGVVQPGRSLRLSCAGSGRTFR | CDR1: |
| VHO4 | NYPMAWFRQAPGKEREFVAGITWVGASTL | NYPMA |
| | YADSAKGRFTISRDNSKNTVYLQMNSLRAE | (SEQ ID NO: 70) |
| | DTAVYSCAAGRGIVAGRIPAEYADWGQGT | CDR2: |
| | LVTVSS (SEQ ID NO: 23) | GITWVGASTLYA |
| | | DSAKG |
| | | (SEQ ID NO: 93) |
| | | CDR3: |
| | | GRGIVAGRIPAEY |
| | | AD |
| | | (SEQ ID NO: 72) |
| CD38 5FIOK | DVQLQESGGGLVQAGGSLRLSCTGSGRTFY | CDR1: |
| VHO5 | LYPMAWFRQAPGKEREFVAGITWVGASTN | LYPMA |
| | YADFAKGRFTISRDNAKNTVYLQMNSLKPE | (SEQ ID NO: 94) |
| | DTAVYSCAAGRGITAGRIPAEYADWGQGT | CDR2: |
| | QVTVSS (SEQ ID NO: 24) | GITWVGASTNYA |
| | | DFAKG |
| | | (SEQ ID NO: 95) |
| | | CDR3: |
| | | GRGITAGRIPAEY |
| | | AD |
| | | (SEQ ID NO: 96) |
| CD38 5FIOK | DVQLQESGGGLVQAGGSLRLSCTGSGRTFR | CDR1: |
| VHO6 | NYPMAWFRQAPGKEREFVAGITWVGASTL | NYPMA |
| | YADFAKGRFTISRDNAKNTVYLQMNSLKPE | (SEQ ID NO: 70) |
| | DTAVYSCAAGRGINAGRIPAEYADWGQGT | CDR2: |
| | QVTVSS (SEQ ID NO: 25) | GITWVGASTLYA |
| | | DFAKG |
| | | (SEQ ID NO: 71) |
| | | CDR3: |
| | | GRGINAGRIPAEY |
| | | AD |
| | | (SEQ ID NO: 97) |
| CD38 5F21 | QMQLVQSGPEVKKPGTSVKVSCKASGLLFR | CDR1: |
| VHO2 | LASMGWYRQARGQERELIGTITVGGKTNY | LASMG |
| | AQKVQERVTITRDMSTSTAYMELSSLRSED | (SEQ ID NO: 73) |
| | TAVYYCNTASPAVGADTWGQGTLVTVSS | CDR2: |
| | (SEQ ID NO: 26) | TITVGGKTNYAQ |
| | | KVQE |
| | | (SEQ ID NO: 98) |
| | | CDR3: |
| | | ASPAVGADT |
| | | (SEQ ID NO: 75) |
| CD38 5F21 | QVTLKESGPVLVKPTETLTLTCTASGLLFRL | CDR1: |
| VHO3 | ASMGWYRQPPGKERELIATITVGGKTNYST | LASMG |
| | SLKSRLTISRDTSKSQVVLTMTNMDPVDTAT | (SEQ ID NO: 73) |
| | YYCNTASPAVGADTWGQGTLVTVSS (SEQ | CDR2: |
| | ID NO: 27) | TITVGGKTNYSTS |
| | | LKS |
| | | (SEQ ID NO: 99) |
| | | CDR3: |
| | | ASPAVGADT |
| | | (SEQ ID NO: 75) |
| CD38 5F21 | EVQLVESGGGLVKPGGSLRLSCAASGLLFRL | CDR1: |
| VHO4 | ASMGWYRQAPGKERELVGTITVGGKTNYA | LASMG |
| | APVKGRFTISRDDSKNTVYLQMNSLKTEDT | (SEQ ID NO: 73) |
| | AVYYCNTASPAVGADTWGQGTLVTVSS | CDR2: |
| | (SEQ ID NO: 28) | TITVGGKTNYAA |
| | | PVKG |
| | | (SEQ ID NO: 100) |
| | | CDR3: |
| | | ASPAVGADT |
| | | (SEQ ID NO: 75) |
| CD38 5F21 | DVQLQESGGGSVQAGGSLTLSCTASGWLFR | CDR1: |
| VHO5 | LASMGWYRQAPGKEREMIAVITTGGRTNY | LASMG |
| | KDSVQGRFIITRDNTKSTVTLQMNRLKPEDT | (SEQ ID NO: 73) |
| | AVYYCNTSMPVVGADEWGQGTRVTVSS | CDR2: |
| | (SEQ ID NO: 29) | VITTGGRTNYKDS |
| | | VQG |
| | | (SEQ ID NO: 101) |
| | | CDR3: |
| | | SMPVVGADE |
| | | (SEQ ID NO: 102) |
| CD38 5F21 | DVQLQESGGGSVQAGGSLTLSCTASGLLFRL | CDR1: |
| VHO6 | ASMGWYRQAPGKERELIAIITKGGKTNYK | LASMG |
| | DSVQGRFIITRDNTKSTVTLQMNRLKPEDTA | (SEQ ID NO: 73) |
| | VYYCNTTSPAVGADWWGQGTRVTVSS | CDR2: |
| | (SEQ ID NO: 30) | IITKGGKTNYKDS |
| | | VQG |
| | | (SEQ ID NO: 103) |
| | | CDR3: |
| | | TSPAVGADW |
| | | (SEQ ID NO: 104) |

TABLE 4

| Description | Amino Acid Sequence | CDRs |
|---|---|---|
| Cris-7-VH0 | QVQLQQSGAELARPGASVKMSCKASGYTFT | CDR1: |
| | RSTMHWVKQRPGQGLEWIGYINPSSAYTN | RSTMH |
| | YNQKFKDKATLTADKSSSTAYMQLSSLTSE | (SEQ ID NO: 105) |
| | DSAVYYCASPQVHYDYNGFPYWGQGTLVT | CDR2: |
| | VSA (SEQ ID NO: 31) | YINPSSAYTNYNQ |
| | | KFKD |
| | | (SEQ ID NO: 106) |
| | | CDR3: |
| | | PQVHYDYNGFPY |
| | | (SEQ ID NO: 107) |
| Cris-7-VH1 | QVQLVQSGAEVKKPGSSVKVSCKASGYTFT | CDR1: |
| | RSTMHWVRQAPGQGLEWIGYINPSSAYTN | RSTMH |
| | YAQKFQGRATLTADESTSTAYMELSSLRSE | (SEQ ID NO: 105) |
| | DTAVYYCASPQVHYDYNGFPYWGQGTLVT | CDR2: |
| | VSS (SEQ ID NO: 32) | YINPSSAYTNYAQ |
| | | KFQG |
| | | (SEQ ID NO: 108) |
| | | CDR3: |
| | | PQVHYDYNGFPY |
| | | (SEQ ID NO: 107) |
| Cris-7-VH2 | EVQLVESGGGLVQPGRSLRLSCTASGYTFTR | CDR1: |
| | STMHWVRQAPGKGLEWVGYINPSSAYTNY | RSTMH |
| | AASFKGRATLSADGSKSIAYLQMNSLKTED | (SEQ ID NO: 105) |
| | TAVYYCASPQVHYDYNGFPYWGQGTLVTV | CDR2: |
| | SS (SEQ ID NO: 33) | YINPSSAYTNYAA |
| | | SFKG |
| | | (SEQ ID NO: 109) |
| | | CDR3: |
| | | PQVHYDYNGFPY |
| | | (SEQ ID NO: 107) |
| Cris-7-VH3 | QVQLVQSGSELKKPGASVKMSCKASGYTFT | CDR1: |
| | RSTMHWVRQAPGQGLEWMGYINPSSAYTN | RSTMH |
| | YAQGFTGRAVLSADTSVSTAYLQLCSLKAE | (SEQ ID NO: 105) |
| | DTAVYYCASPQVHYDYNGFPYWGQGTLVT | CDR2: |
| | VSS (SEQ ID NO: 34) | YINPSSAYTNYAQ |
| | | GFTG |
| | | (SEQ ID NO: 110) |
| | | CDR3: |
| | | PQVHYDYNGFPY |
| | | (SEQ ID NO: 107) |
| cris-7-VL0 | QVVLTQSPAIMSAFPGEKVTMTCSASSSVSY | CDR1: |
| | MNWYQQKSGTSPKRWIYDSSKLASGVPARF | SASSSVSYMN |
| | SGSGSGTSYSLTISSMETEDAATYYCQQWSR | (SEQ ID NO: 111) |
| | NPPTFGGGTKLQIT (SEQ ID NO: 35) | CDR2: |
| | | DSSKLAS |
| | | (SEQ ID NO: 112) |
| | | CDR3: |
| | | QQWSRNPPT |
| | | (SEQ ID NO: 113) |
| cris-7-VL1 | DIQMTQSPSSLSASVGDRVTITCRASSSVSY | CDR1: |
| | MNWYQQKPGKAPKRLIYDSSKLASGVPSRF | RASSSVSYMN |
| | SGSGSGTEFTLTISSLQPEDFATYYCQQWSR | (SEQ ID NO: 114) |
| | NPPTFGQGTKLEIK (SEQ ID NO: 36) | CDR2: |
| | | DSSKLAS |
| | | (SEQ ID NO: 112) |
| | | CDR3: |
| | | QQWSRNPPT |
| | | (SEQ ID NO: 113) |
| cris-7-VL2 | AIRLTQSPFSLSASVGDRVTITCSASSSVSYM | CDR1: |
| | NWYQQKPAKAPKRWIYDSSKLASGVPSRFS | SASSSVSYMN |
| | GSGSGTDYTLTISSLQPEDAATYYCQQWSR | (SEQ ID NO: 111) |
| | NPPTFGQGTKLEIK (SEQ ID NO: 37) | CDR2: |
| | | DSSKLAS |
| | | (SEQ ID NO: 112) |
| | | CDR3: |
| | | QQWSRNPPT |
| | | (SEQ ID NO: 113) |
| SP34 v8 LC | QTVVTQEPSLTVSPGGTVTLTCRSSTGAVTT | CDR1: |
| | SNYANWVQQKPGQAPRGLIGGTNKRAPGT | RSSTGAVTTSNY |
| | PARFSGSLLGGKAALTLSGVQPEDEAEYYCA | AN |
| | LWYSNLWVFGGGTKLTVL | (SEQ ID NO: 115) |
| | (SEQ ID NO: 38) | CDR2: |
| | | GTNKRAP |
| | | (SEQ ID NO: 116) |
| | | CDR3: |
| | | ALWYSNLWV |
| | | (SEQ ID NO: 117) |
| SP34 v8 HC | EVQLVESGGGLVQPGGSLKLSCAASGFTFN | CDR1: |
| | TYAMNWVRQAPGKGLEWVARIRSKYNNY | GFTFNTYAMN |
| | ATYYADSVKDRFTISRDDSKNTAYLQMNNL | (SEQ ID NO: 118) |
| | KTEDTAVYYCVRHGNFGNSYVAWFAYWG | CDR2: |
| | QGTLVTVSS (SEQ ID NO: 39) | RIRSKYNNYATY |
| | | (SEQ ID NO: 119) |
| | | CDR3: |
| | | HGNFGNSYVAWF |
| | | AY |
| | | (SEQ ID NO: 120) |
| 40G5 LC | DIVMTQSPDSLAVSLGERATINCKSSQSLLN | CDR1: |
| | SRTRKNYLAWYQQKPGQPPKLLIYWASTR | KSSQSLLNSRTRK |
| | ESGVPDRFSGSGSGTDFTLTISSLQAEDVAVY | NYLA |
| | YCTQSFILRTFGQGTKVEIK | (SEQ ID NO: 121) |
| | (SEQ ID NO: 40) | CDR2: |
| | | WASTRES |
| | | (SEQ ID NO: 122) |
| | | CDR3: |
| | | TQSFILRT |
| | | (SEQ ID NO: 123) |
| 40G5 HC | EVQLVQSGAEVKKPGASVKVSCKASGYTFT | CDR1: |
| | NYYIHWVRQAPGQGLEWIGWIYPGDGNTK | NYYIH |
| | YNEKFKGRATLTADTSTSTAYLELSSLRSED | (SEQ ID NO: 124) |
| | TAVYYCARDSYSNYYFDYWGQGTLVTVSS | CDR2: |
| | (SEQ ID NO: 41) | WIYPGDGNTKYN |
| | | EKFKG |
| | | (SEQ ID NO: 125) |
| | | CDR3: |
| | | DSYSNYYFDY |
| | | (SEQ ID NO: 126) |

In some embodiment, the present disclosure provides an isolated anti-CD38 monoclonal antibody, or an antigen-binding portion thereof, an antibody fragment, or an antibody mimetic that binds an epitope on human CD38 recognized by an antibody comprising a heavy chain variable region comprising an amino acid sequence selected from SEQ ID NOs: 3 and 12-16 and a light chain variable region comprising an amino acid sequence selected from SEQ ID NOs: 2 and 7-11.

In some embodiments, the present disclosure provides an anti-CD38 antibody selected from the group consisting of a single domain VHH, a single domain VHO (Variable Heavy Only) domain comprising a HC (heavy chain) variable region comprising an amino acid sequence selected from SEQ ID NOs: 4-6 and 17-30, an Affibody, a DARPin, an Anticalin, an Avimer, a Versabody, and a Duocalin.

In some embodiments, the present disclosure provides a composition comprising an isolated anti-CD38 antibody, or antigen-binding portion, an antibody fragment, or an antibody mimetic disclosed herein, and a pharmaceutically acceptable carrier.

In some embodiments, the disclosure provides an isolated nucleic acid molecule encoding the heavy or light chain of an anti-CD38 isolated antibody or antigen-binding portion, or an antibody fragment that binds an epitope on human CD38. In some embodiments, the disclosure provides expression vectors comprising such nucleic acid molecules, and host cells comprising such expression vectors.

In some embodiments, the present disclosure provides a method for preparing an anti-CD38 antibody, said method comprising the steps of obtaining a host cell that contains one or more nucleic acid molecules encoding the anti-CD38 antibody; growing the host cell in a host cell culture; providing host cell culture conditions wherein the one or more nucleic acid molecules are expressed; and recovering the antibody from the host cell or from the host cell culture.

Also described is an isolated anti-CD38 monoclonal antibody or an antigen binding portion thereof, an antibody fragment, or an antibody mimetic that binds an epitope on human CD38 recognized by an antibody comprising a heavy chain variable region and a light chain variable region selected from the group consisting of the heavy chain variable region amino acid sequence set forth in SEQ ID NOs: 3 and 12-16 and the light chain variable region amino acid sequence set forth in SEQ ID NOs: 2 and 7-11; and the heavy chain single domain variable region amino acid sequence set forth in SEQ ID NOs: 4-6 and 17-30.

Also described herein is an isolated monoclonal antibody, or an antigen binding portion thereof, which binds an epitope on a CD38 polypeptide having an amino acid sequence of SEQ ID NO: 1 recognized by an antibody comprising a heavy chain variable region comprising an amino acid sequence selected from SEQ ID NO: 3 and a light chain variable region comprising an amino acid sequence set forth in SEQ ID NO: 2. Also described herein is an isolated monoclonal single domain antibody, or an antigen binding portion thereof, which binds an epitope on a CD38 polypeptide having an amino acid sequence of SEQ ID NO: 1 recognized by an antibody comprising a heavy chain variable region comprising an amino acid sequence selected from SEQ ID NOs: 4-6 and 17-30.

Also described is an isolated monoclonal antibody or an antigen binding portion thereof, an antibody fragment, or an antibody mimetic that binds an epitope on human CD38 recognized by an antibody comprising a heavy chain variable region and a light chain variable region selected from the group consisting of the heavy chain variable region amino acid sequence set forth in SEQ ID NO: 12 and the light chain variable region amino acid sequence set forth in SEQ ID NO: 7; the heavy chain variable region amino acid sequence set forth in SEQ ID NO: 12 and the light chain variable region amino acid sequence set forth in SEQ ID NO: 8; the heavy chain variable region amino acid sequence set forth in SEQ ID NO: 12 and the light chain variable region amino acid sequence set forth in SEQ ID NO: 9; the heavy chain variable region amino acid sequence set forth in SEQ ID NO: 12 and the light chain variable region amino acid sequence set forth in SEQ ID NO:10; the heavy chain variable region amino acid sequence set forth in SEQ ID NO: 12 and the light chain variable region amino acid sequence set forth in SEQ ID NO: 11; the heavy chain variable region amino acid sequence set forth in SEQ ID NO: 13 and the light chain variable region amino acid sequence set forth in SEQ ID NO: 7; the heavy chain variable region amino acid sequence set forth in SEQ ID NO: 13 and the light chain variable region amino acid sequence set forth in SEQ ID NO: 8; the heavy chain variable region amino acid sequence set forth in SEQ ID NO: 13 and the light chain variable region amino acid sequence set forth in SEQ ID NO: 9; the heavy chain variable region amino acid sequence set forth in SEQ ID NO: 13 and the light chain variable region amino acid sequence set forth in SEQ ID NO: 10; the heavy chain variable region amino acid sequence set forth in SEQ ID NO: 13 and the light chain variable region amino acid sequence set forth in SEQ ID NO: 11; the heavy chain variable region amino acid sequence set forth in SEQ ID NO: 14 and the light chain variable region amino acid sequence set forth in SEQ ID NO: 7; the heavy chain variable region amino acid sequence set forth in SEQ ID NO: 14 and the light chain variable region amino acid sequence set forth in SEQ ID NO: 8; the heavy chain variable region amino acid sequence set forth in SEQ ID NO: 14 and the light chain variable region amino acid sequence set forth in SEQ ID NO: 9; and the heavy chain variable region amino acid sequence set forth in SEQ ID NO: 14 and the light chain variable region amino acid sequence set forth in SEQ ID NO: 10; the heavy chain variable region amino acid sequence set forth in SEQ ID NO: 14 and the light chain variable region amino acid sequence set forth in SEQ ID NO: 11; the heavy chain variable region amino acid sequence set forth in SEQ ID NO: 15 and the light chain variable region amino acid sequence set forth in SEQ ID NO: 7; the heavy chain variable region amino acid sequence set forth in SEQ ID NO: 15 and the light chain variable region amino acid sequence set forth in SEQ ID NO: 8; the heavy chain variable region amino acid sequence set forth in SEQ ID NO: 15 and the light chain variable region amino acid sequence set forth in SEQ ID NO: 9; the heavy chain variable region amino acid sequence set forth in SEQ ID NO: 15 and the light chain variable region amino acid sequence set forth in SEQ ID NO: 10; the heavy chain variable region amino acid sequence set forth in SEQ ID NO: 15 and the light chain variable region amino acid sequence set forth in SEQ ID NO: 11; the heavy chain variable region amino acid sequence set forth in SEQ ID NO: 16 and the light chain variable region amino acid sequence set forth in SEQ ID NO: 7; the heavy chain variable region amino acid sequence set forth in SEQ ID NO: 16 and the light chain variable region amino acid sequence set forth in SEQ ID NO: 8; the heavy chain variable region amino acid sequence set forth in SEQ ID NO: 16 and the light chain variable region amino acid sequence set forth in SEQ ID NO: 9; the heavy chain variable region amino acid sequence set forth in SEQ ID NO: 16 and the light chain variable region amino acid sequence set forth in SEQ ID NO: 10; and the heavy chain variable region amino acid sequence set forth in SEQ ID NO: 16 and the light chain variable region amino acid sequence set forth in SEQ ID NO: 11.

Also described is an isolated monoclonal antibody or an antigen binding portion thereof, an antibody fragment, or an antibody mimetic that binds an epitope on human CD3 recognized by an antibody comprising a heavy chain variable region and a light chain variable region selected from the group consisting of the heavy chain and light chain variable region amino acid sequence set forth in of SEQ ID NO: 31 and SEQ ID NO: 35; SEQ ID NO: 31 and SEQ ID NO: 36; SEQ ID NO: 31 and SEQ ID NO: 37; SEQ ID NO: 32 and SEQ ID NO: 35; SEQ ID NO: 32 and SEQ ID NO: 36; SEQ ID NO: 32 and SEQ ID NO: 37; SEQ ID NO: 33 and SEQ ID NO: 35; SEQ ID NO: 33 and SEQ ID NO: 36; SEQ ID NO: 33 and SEQ ID NO: 37; SEQ ID NO: 34 and SEQ ID NO: 35; SEQ ID NO: 34 and SEQ ID NO: 36; SEQ ID NO: 34 and SEQ ID NO: 37; SEQ ID NO: 39 and SEQ ID NO: 38; SEQ ID NO: 41 and SEQ ID NO: 40.

In some embodiments, the present disclosure provides an isolated nucleic acid molecule encoding the heavy or light chain of the isolated anti-CD38 or anti-CD3 antibody or antigen binding portion thereof of the disclosure, and in further aspects may include an expression vector comprising such nucleic acids, and host cells comprising such expression vectors.

Another embodiment of the present disclosure is a hybridoma expressing the anti-CD38 or anti-CD3 antibody or antigen binding portion thereof of the disclosure.

Other aspects of the disclosure are directed to methods of making the anti-CD38 or anti-CD3 antibodies of the disclosure, comprising the steps of immunizing an animal with a CD38 peptide or CD3 peptide; or CD38 protein or CD3 protein; or CD38 domain or CD3 domain, recovering mRNA from the B cells of said animal; and converting said mRNA to cDNA.

In some embodiments, the present disclosure provides a method for preparing an anti-CD38 antibody or an anti-CD3 antibody, said method comprising the steps of obtaining a host cell that contains one or more nucleic acid molecules encoding the anti-CD38 antibody or an anti-CD3 antibody of this disclosure; growing the host cell in a host cell culture; providing host cell culture conditions wherein the one or more nucleic acid molecules are expressed; and recovering the antibody from the host cell or from the host cell culture.

In some embodiments, the present disclosure provides a method of expressing cDNA encoding anti-CD38 or anti-CD3 monoclonal antibodies or an antigen binding portion thereof, an antibody fragment, or an antibody mimetic expressing said cDNA in phages such that the anti-CD38 antibodies or anti-CD3 antibodies encoded by said cDNA are presented on the surface of said phages; selecting phages that present anti-CD38 antibodies or anti-CD3 antibodies; recovering nucleic acid molecules from said selected phages that encode said anti-CD38 immunoglobulins and said anti-CD3 immunoglobulins; expressing said recovered nucleic acid molecules in a host cell; and recovering antibodies from said host cell that bind CD38 and CD3, respectively.

As non-limiting examples, the disclosure provides for the preparation of the CD3 binding constructs wherein a host cell can be co-transfected with nucleic acids encoding the following pairings of SEQ ID NO: 31 and SEQ ID NO: 35; SEQ ID NO: 31 and SEQ ID NO: 36; SEQ ID NO: 31 and SEQ ID NO: 37; SEQ ID NO: 32 and SEQ ID NO: 35; SEQ ID NO: 32 and SEQ ID NO: 36; SEQ ID NO: 32 and SEQ ID NO: 37; SEQ ID NO: 33 and SEQ ID NO: 35; SEQ ID NO: 33 and SEQ ID NO: 36; SEQ ID NO: 33 and SEQ ID NO: 37; SEQ ID NO: 34 and SEQ ID NO: 35; SEQ ID NO: 34 and SEQ ID NO: 36; SEQ ID NO: 34 and SEQ ID NO: 37; SEQ ID NO: 38 and SEQ ID NO: 39; or SEQ ID NO: 40 and SEQ ID NO: 41.

As non-limiting examples, the disclosure provides for the CD38 binding constructs wherein a host cell can be co-transfected with nucleic acids encoding the following: SEQ ID NO: 2 and SEQ ID NO: 3; SEQ ID NO: 4 alone; SEQ ID NO: 5 alone, and SEQ ID NO: 6 alone; SEQ ID NO: 7 and SEQ ID NO: 12; SEQ ID NO: 7 and SEQ ID NO: 13; SEQ ID NO: 7 and SEQ ID NO: 14; SEQ ID NO: 7 and SEQ ID NO: 15; SEQ ID NO: 7 and SEQ ID NO: 16; SEQ ID NO: 31 and SEQ ID NO: 37; SEQ ID NO: 8 and SEQ ID NO: 12; SEQ ID NO: 8 and SEQ ID NO: 13; SEQ ID NO: 8 and SEQ ID NO: 14; SEQ ID NO: 8 and SEQ ID NO: 15; SEQ ID NO: 8 and SEQ ID NO: 16; SEQ ID NO: 9 and SEQ ID NO: 12; SEQ ID NO: 9 and SEQ ID NO: 13; SEQ ID NO: 9 and SEQ ID NO: 14; SEQ ID NO: 9 and SEQ ID NO: 15; SEQ ID NO: 9 and SEQ ID NO: 16; SEQ ID NO: 10 and SEQ ID NO: 12; SEQ ID NO: 10 and SEQ ID NO: 13; SEQ ID NO: 10 and SEQ ID NO: 14; SEQ ID NO: 10 and SEQ ID NO: 15; SEQ ID NO: 10 and SEQ ID NO: 16; SEQ ID NO: 11 and SEQ ID NO: 12; SEQ ID NO: 11 and SEQ ID NO: 13; SEQ ID NO: 11 and SEQ ID NO: 14; SEQ ID NO: 11 and SEQ ID NO: 15; or SEQ ID NO: 11 and SEQ ID NO: 16. The CD38 VHO arm can be expressed alone as any one of SEQ ID NOS: 17-30. The CD38 binding arm can have tandem sequences of SEQ ID NO: 4-linker-SEQ ID NO: 5; SEQ ID NO: 4-linker-SEQ ID NO: 6, SEQ ID NO: 5-linker SEQ ID NO: 4, SEQ ID NO: 5-linker-SEQ ID NO: 6, SEQ ID NO: 6 linker-SEQ ID NO: 4, SEQ ID NO: 6-linker SEQ ID NO: 5, as well as any pair of sequences selected from SEQ ID NO. 17-30.

Some embodiments provide a bispecific antibody comprising antibody sequences targeting CD3 selected from heavy chain variable regions comprising an amino acid sequence set forth in SEQ ID NOs: 31 to 34; and light chain variable regions comprising an amino acid sequence set forth in SEQ ID NOs: 35 to 40; and antibody sequences targeting CD38 selected from amino acid sequences set forth in SEQ ID NOs: 2-30.

### Fc Region of Anti-CD38, Anti-CD3, and CD3×CD38 Bispecific Antibodies

The anti-CD38, anti-CD3, and CD3×CD38 bispecific antibodies disclosed herein may comprise a modified F_{c} region, wherein the modified F_{c} region comprises at least one amino acid modification relative to a native F_{c} region, for example, to extend the half-life of the bispecific antibody, enhance resistance of the bispecific antibody to proteolytic degradation, reduce effector functionality of the bispecific antibody, facilitate generation of the bispecific antibody by F_{c} heterodimerization, facilitate the multimerization of the bispecific antibody, and/or improve manufacturing and drug stability of the bispecific antibody.

In some embodiments, the Fc domain is altered to allow for silencing of the Fc domain to minimize effector function activity which can cause immune cell depletion and cytokine release syndrome.

In some embodiments, anti-CD38, anti-CD3, and CD3×CD38 bispecific antibodies as described herein are provided with a modified F_{c} region wherein a naturally occurring F_{c} region is modified to extend the half-life of the antibody when compared to the parental native antibody in a biological environment, for example, the serum half-life or a half-life measured by an in vitro assay. Exemplary mutations that may be made singularly or in combination are T250Q, M252Y, I253A, S254T, T256E, P2571, T307A, D376V, E380A, M428L, H433K, N434S, N434A, N434H, N434F, H435A, and H435R mutations.

In certain embodiments, the extension of half-life can be realized by engineering the M252Y/S254T/T256E mutations in IgG1 F_{c} residue numbering according to the EU Index (Dall'Acqua, Kiener et al. 2006).

In certain embodiments, the extension of half-life can also be realized by engineering the M428L/N434S mutations in IgG₁ F_{c} (Zalevsky, Chamberlain et al. 2010).

In certain embodiments, the extension of half-life can also be realized by engineering the T250Q/M428L mutations in IgG₁ F_{c} (Hinton, Xiong et al. 2006).

In certain embodiments, the extension of half-life can also be realized by engineering the N434A mutations in IgG₁ F_{c} (Shields, Namenuk et al. 2001).

In certain embodiments, the extension of half-life can also be realized by engineering the T307A/E380A/N434A mutations in IgG₁ F_{c} (Petkova, Akilesh et al. 2006).

The effect F_{c} engineering on the extension of antibody half-life can be evaluated in PK studies in mice relative to antibodies with native IgG F_{c}.

In some embodiments, the anti-CD38, anti-CD3, and CD3×CD38 bispecific antibodies as described herein are provided with a modified F_{c} region wherein a naturally occurring F_{c} region is modified to enhance the antibody resistance to proteolytic degradation by a protease that cleaves the wild-type antibody between or at residues 222-237 (EU numbering).

In certain embodiments, the resistance to proteolytic degradation can be realized by engineering E233P/L234A/L235A mutations in the hinge region with G236 deleted when compared to a parental native antibody, residue numbering according to the EU Index (Kinder, Greenplate et al. 2013).

In instances where effector functionality is not desired, the antibodies of the disclosure may further be engineered to introduce at least one mutation in the antibody F_{c} that reduces binding of the antibody to an activating F_{c}γ receptor (F_{c}γR) and/or reduces F_{c} effector functions such as C1q binding, complement dependent cytotoxicity (CDC), antibody-dependent cell-mediated cytotoxicity (ADCC) or phagocytosis (ADCP).

F_{c} positions that may be mutated to reduce binding of the antibody to the activating F_{c}γR and subsequently to reduce effector functions are those described for example in (Xu, Alegre et al. 2000) (Vafa, Gilliland et al. 2014) (Bolt, Routledge et al. 1993, Shields, Namenuk et al. 2001, Chu, Vostiar et al. 2008). Fc mutations with minimal ADCC, ADCP, CDC, Fc mediated cellular activation have been described also as sigma mutations for IgG1, IgG2 and IgG4 (Tam, McCarthy et al. 2017). Exemplary mutations that may be made singularly or in combination are K214T, E233P, L234V, L234A, deletion of G236, V234A, F234A, L235A, G237A, P238A, P238S, D265A, S267E, H268A, H268Q, Q268A, N297A, A327Q, P329A, D270A, Q295A, V309L, A327S, L328F, A330S and P331S mutations on IgG₁, IgG₂, IgG₃ or IgG₄.

Exemplary combination mutations that may be made to reduce ADCC are L234A/L235A on IgG₁, V234A/G237A/P238S/H268A/V309L/A330S/P331S on IgG₂, F234A/L235A on IgG₄, S228P/F234A/L235A on IgG₄, N297A on IgG₁, IgG₂, IgG₃ or IgG₄, V234A/G237A on IgG₂, K214T/E233P/L234V/L235A/G236 deleted/A327G/P331A/D365E/L358M on IgG₁, H268Q/V309L/A330S/P331S on IgG₂, S267E/L328F on IgG₁, L234F/L235E/D265A on IgG₁, L234A/L235A/G237A/P238S/H268A/A330S/P331S on IgG₁, S228P/F234A/L235A/G237A/P238S on IgG₄, and S228P/F234A/L235A/G236-deleted/G237A/P238S on IgG₄. Hybrid IgG_{2/4} F_{c} domains may also be used, such as F_{c} with residues 117-260 from IgG₂ and residues 261-447 from IgG₄.

In some embodiments, the CD3×CD38 bispecific antibody is provided with a modified F_{c} region wherein a naturally occurring F_{c} region is modified to facilitate the generation of bispecific antibody by Fc heterodimerization.

In certain embodiments, the Fc heterodimerization can be realized by engineering F405L and K409R mutations on two parental antibodies and the generation of bispecific antibody in a process known as Fab arm exchange (Labrijn, Meesters et al. 2014).

In certain embodiments, the Fc heterodimerization can also be realized by F_{c} mutations to facilitate Knob-in-Hole strategy (see, e.g., Intl. Publ. No. WO 2006/028936). An amino acid with a small side chain (hole) is introduced into one Fc domain and an amino acid with a large side chain (knob) is introduced into the other Fc domain. After co-expression of the two heavy chains, a heterodimer is formed as a result of the preferential interaction of the heavy chain with a “hole” with the heavy chain with a “knob” (Ridgway, Presta et al. 1996). Exemplary Fc mutation pairs forming a knob and a hole are: T366Y/F405A, T366W/F405W, F405W/Y407A, T394W/Y407T, T394S/Y407A, T366W/T394S, F405W/T394S and T366W/T366S/L368A/Y407V.

In certain embodiments, the Fc heterodimerization can also be realized by Fc mutations to facilitate the electrostatically-matched interactions strategy (Gunasekaran, Pentony et al. 2010). Mutations can be engineered to generate positively charged residues at one Fc domain and negatively charged residues at the other Fc domain as described in US Patent Publ. No. US2010/0015133; US Patent Publ. No. US2009/0182127; US Patent Publ. No. US2010/028637 or US Patent Publ. No. US2011/0123532. Heavy chain heterodimerization can be formed by electrostatically matched interactions between two mutated Fc.

In some embodiments, the present CD3×CD38 bispecific antibody is provided with a modified F_{c} region wherein a naturally occurring Fc region is modified to facilitate the multimerization of the antibody upon interaction with cell surface receptors, although the bispecific antibody ordinarily exists as monomer in serum and in solution. The F_{c} mutations that facilitate antibody multimerization include, but are not limited to, E345R mutation, E430G mutation, E345R/E430G mutations, E345R/E430G/Y440R mutations as described in (Diebolder, Beurskens et al. 2014). Such mutations may also include, but are not limited to, T437R mutation, T437R/K248E mutations, and T437R/K338A mutations as described in (Zhang, Armstrong et al. 2017).

### Antibody Modifications

Antibodies further comprising conservative modifications are within the scope of the disclosure. “Conservative modifications” refer to amino acid modifications that do not significantly affect or alter the binding characteristics of the antibody containing the amino acid sequences. Conservative modifications include amino acid substitutions, additions, and deletions. Conservative substitutions are those in which the amino acid is replaced with an amino acid residue having a similar side chain. The families of amino acid residues having similar side chains are well defined and include amino acids with acidic side chains (e.g., aspartic acid, glutamic acid), basic side chains (e.g., lysine, arginine, histidine), nonpolar side chains (e.g., alanine, valine, leucine, isoleucine, proline, phenylalanine, methionine), uncharged polar side chains (e.g., glycine, asparagine, glutamine, cysteine, serine, threonine, tyrosine, tryptophan), aromatic side chains (e.g., phenylalanine, tryptophan, histidine, tyrosine), aliphatic side chains (e.g., glycine, alanine, valine, leucine, isoleucine, serine, threonine), amide (e.g., asparagine, glutamine), beta-branched side chains (e.g., threonine, valine, isoleucine) and sulfur-containing side chains (cysteine, methionine). Furthermore, any native residue in the polypeptide may also be substituted with alanine, as has been previously described for alanine scanning mutagenesis. Amino acid substitutions to the antibodies of the disclosure may be made by known methods for example by PCR mutagenesis (U.S. Pat. No. 4,683,195). Alternatively, libraries of variants may be generated for example using random (NNK) or non-random codons, for example DVK codons, which encode 11 amino acids (Ala, Cys, Asp, Glu, Gly, Lys, Asn, Arg, Ser, Tyr, Trp). The resulting antibody variants may be evaluated for their characteristics using assays described herein.

The antibodies of this disclosure may be post-translationally modified by processes such as glycosylation, isomerization, deglycosylation or non-naturally occurring covalent modification such as the addition of polyethylene glycol moieties (pegylation) and lipidation. Such modifications may occur in vivo or in vitro. For example, the antibodies of the disclosure may be conjugated to polyethylene glycol (PEGylated) to improve their pharmacokinetic profiles. Conjugation may be conducted by techniques known to those skilled in the art. Conjugation of therapeutic antibodies with PEG has been shown to enhance pharmacodynamics while not interfering with function.

Antibodies of this disclosure may be modified to improve stability, selectivity, cross-reactivity, affinity, immunogenicity or other desirable biological or biophysical property are within the scope of the disclosure. Stability of an antibody is influenced by a number of factors, including (1) core packing of individual domains that affects their intrinsic stability, (2) protein/protein interface interactions that have impact upon the HC and LC pairing, (3) burial of polar and charged residues, (4) H-bonding network for polar and charged residues; and (5) surface charge and polar residue distribution among other intra- and inter-molecular forces (Worn and Pluckthun 2001). Potential structure destabilizing residues may be identified based upon the crystal structure of the antibody or by molecular modelling in certain cases, and the effect of the residues on antibody stability may be assessed by generating and evaluating variants harboring mutations in the identified residues. One of the ways to increase antibody stability is to raise the thermal transition midpoint (Tₘ) as measured by differential scanning calorimetry (DSC). In general, the protein Tₘ is correlated with its stability and inversely correlated with its susceptibility to unfolding and denaturation in solution and the degradation processes that depend on the tendency of the protein to unfold. A number of studies have found correlation between the ranking of the physical stability of formulations measured as thermal stability by DSC and physical stability measured by other methods. Formulation studies suggest that a Fab Tₘ has implication for long-term physical stability of a corresponding mAb.

Antibodies of this disclosure may have amino acid substitutions in the F_{c} region that improve manufacturing and drug stability. An example for IgG₁ is H224S (or H224Q) in the hinge 221-DKTHTC-226 (EU numbering) which blocks radically induced cleavage; and for IgG₄, the S228P mutation blocks half-antibody exchange.

### Shielding or Masking Domains

As non-limiting examples, the disclosure provides a bispecific antibody comprising a shielding domain (also referred to as masking domain, mask, or cap) selected from the shielding domain amino acid sequences set forth as SEQ ID NO: 42-45 in Table 5 that can shield binding of the CD38 Fabs and the shielding domain amino acid sequences set forth as SEQ ID NO: 46-52 in Table 5 that can shield binding of the CD3 Fabs. Some embodiments provide various shielding or caps that mask CD38 binding as noted in SEQ ID NO: 42-51. Some embodiments provide various shielding or caps that mask CD3 binding as noted in SEQ ID NO: 46-52.

TABLE 5

| SEQ | | |
|---|---|---|
| ID | | |
| NO: | Description | Amino Acid Sequence |
| 42 | K VHO1 | MAWVWTLLFLMAAAQSIQAQVQLQESGGGLVQAGGSLRLSCAVSGNTI |
| | | SRYATGWFRQTPGNEREFVAAIRWINGNTYYADSVEGRFTISRDSGKNT |
| | | VYLQMNNLQPEDTAVYYCASRFLPYASSNAYHEALYNYDYWGQGTQV |
| | | TVSSGGGGSDKTHTCPPCPAPEAAGGPSVFLFPPKPKDTLMISRTPEVTCV |
| | | VVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSREEM |
| | | TKNQVSLTCLVKGFYPSDIAVEWESNGQPENNYKTTPPVLDSDGSFFLYS |
| | | RLTVDKSRWQQGNVFSCSVMHEALHNHYTQKSLSLSPGK |
| 43 | K VHO2 | MAWVWTLLFLMAAAQSIQAQVQLQESGGGLVQAGGSLRLSCAVSGNTI |
| | | SRYATGWFRQTPGNEREFVAAIRWINGNTYYADSVEGRFTISRDSGKNT |
| | | VYLQMNNLQPEDTAVYYCASRFLPYASSNAYHESLYNYDYWGQGTQV |
| | | TVSSGGGGSDKTHTCPPCPAPEAAGGPSVFLFPPKPKDTLMISRTPEVTCV |
| | | VVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSREEM |
| | | TKNQVSLTCLVKGFYPSDIAVEWESNGQPENNYKTTPPVLDSDGSFFLYS |
| | | RLTVDKSRWQQGNVFSCSVMHEALHNHYTQKSLSLSPGK |
| 44 | K VHO3 | MAWVWTLLFLMAAAQSIQAQVQLQESGGGLVQAGGSLRLSCAVSGNTI |
| | | SRYATGWFRQTPGNEREFVAAIRWENGNTYYADSVEGRFTISRDSGKNT |
| | | VYLQMNNLQPEDTAVYYCASRFLPYASSNAYHEALYNYDYWGQGTQV |
| | | TVSSGGGGSDKTHTCPPCPAPEAAGGPSVFLFPPKPKDTLMISRTPEVTCV |
| | | VVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSREEM |
| | | TKNQVSLTCLVKGFYPSDIAVEWESNGQPENNYKTTPPVLDSDGSFFLYS |
| | | RLTVDKSRWQQGNVFSCSVMHEALHNHYTQKSLSLSPGK |
| 45 | K VHO4 | MAWVWTLLFLMAAAQSIQAQVQLQESGGGLVQAGGSLRLSCAVSGNTI |
| | | SRYATGWFRQTPGNEREFVAAIRWENGNTYYADSVEGRFTISRDSGKNT |
| | | VYLQMNNLQPEDTAVYYCASRFLPYASSNAYHESLYNYDYWGQGTQV |
| | | TVSSGGGGSDKTHTCPPCPAPEAAGGPSVFLFPPKPKDTLMISRTPEVTCV |
| | | VVDVSHEDPEVKFNWYVDGVEVHNAKTKPREEQYNSTYRVVSVLTVL |
| | | HQDWLNGKEYKCKVSNKALPAPIEKTISKAKGQPREPQVYTLPPSREEM |
| | | TKNQVSLTCLVKGFYPSDIAVEWESNGQPENNYKTTPPVLDSDGSFFLYS |
| | | RLTVDKSRWQQGNVFSCSVMHEALHNHYTQKSLSLSPGK |
| 46 | CH1 | QVQLQESGGGLVQAGGSLRLSCAVSGNTISRYATGWFRQTPGNEREFVA |
| | VHO1 | AIRWTNGNTYYADSVEGRFTISRDSGKNTVYLQRFLPYASSNAYHEALY |
| | | NYDYWGQGTQVTVSSGGGGS |
| | | DKTHTCPPCPAPELLGGPSVFLFPPKPKDTLMISRTPEVTCVVVDVSHEDP |
| | | EVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCL |
| | | VKGFYPSDIAVEWESNGQPENNYKTTPPVLDSDGSFLLYSKLTVDKSRW |
| | | QQGNVFSCSVMHEALHNHYTQKSLSLSPGK |
| 47 | CH1 | QVQLQESGGGLVQAGGSLRLSCAVSGNTISRYATGWFRQTPGNEREFVA |
| | VHO2 | AIRWENGNTYYADSVEGRFTISRDSGKNTVYLQRFLPYASSNAYHESLY |
| | | NYDYWGQGTQVTVSSGGGGS |
| | | DKTHTCPPCPAPELLGGPSVFLFPPKPKDTLMISRTPEVTCVVVDVSHEDP |
| | | EVKFNWYVDGVEVHNAKTKPREEQYNSTYRVVSVLTVLHQDWLNGKE |
| | | YKCKVSNKALPAPIEKTISKAKGQPREPQVYTLPPSREEMTKNQVSLTCL |
| | | VKGFYPSDIAVEWESNGQPENNYKTTPPVLDSDGSFLLYSKLTVDKSRW |
| | | QQGNVFSCSVMHEALHNHYTQKSLSLSPGK |
| 48 | CH1 | QVQLQESGGGLVQAGGSLRLSCAVSGNTLSRYAMGWFRQAPGNEREFV |
| | VHO3 | AAIRWNNGNTHYADSVKGRFTISRDSAKNTVYLQMNNLQPEDTAVYYC |
| | | ASRFLPYASSNAYHEALYNYDYWGQGTQVTVSS |
| | | GGGGS |
| | | DKTHTCPPCPAPEAAGGPSVFLFPPKPKDTLMISRTPEVTCVVVDVSHED |
| | | PEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSRLTVDKSR |
| | | WQQGNVFSCSVMHEALHNHYTQKSLSLSPGK |
| 49 | CH1 | EVOLVESGGGLVQPGGSLRLSCAASGNTLSRYAMGWFRQAPGKEREFV |
| | VHO4 | AAIRWNNGNTHYVDSVKGRFTISRDNAKNSVYLQMNSLRAEDTAVYYC |
| | | ASRFLPYASSNAYHEALYNYDYWGQGTLVTVSS |
| | | GGGGS |
| | | DKTHTCPPCPAPEAAGGPSVFLFPPKPKDTLMISRTPEVTCVVVDVSHED |
| | | PEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSRLTVDKSR |
| | | WQQGNVFSCSVMHEALHNHYTQKSLSLSPGK |
| 50 | CH1 | QVTLKESGPVLVKPTETLTLTCTVSGNTLSRYAMGWFRQAPGKEREFVA |
| | VHO5 | AIRWNNGNTHYSTSLKSRLTISKDTSKSQVVLTMTNMDPVDTATYYCAS |
| | | RFLPYASSNAYHEALYNYDYWGQGTLVTVSS |
| | | GGGGS |
| | | DKTHTCPPCPAPEAAGGPSVFLFPPKPKDTLMISRTPEVTCVVVDVSHED |
| | | PEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSRLTVDKSR |
| | | WQQGNVFSCSVMHEALHNHYTQKSLSLSPGK |
| 51 | CH1 | QVQLVQSGAEVKKPGASVKVSCKASGNTLSRYAMGWFRQAPGQEREF |
| | VHO6 | VAAIRWNNGNTHYAQKFQGRVTMTRDTSTSTVYMELSSLRSEDTAVYY |
| | | CASRFLPYASSNAYHEALYNYDYWGQGTLVTVSS |
| | | GGGGS |
| | | DKTHTCPPCPAPEAAGGPSVFLFPPKPKDTLMISRTPEVTCVVVDVSHED |
| | | PEVKFNWYVDGVEVHNAKTKPREEQYNSTYRVVSVLTVLHQDWLNGK |
| | | EYKCKVSNKALPAPIEKTISKAKGQPREPQVYTLPPSREEMTKNQVSLTC |
| | | LVKGFYPSDIAVEWESNGQPENNYKTTPPVLDSDGSFFLYSRLTVDKSR |
| | | WQQGNVFSCSVMHEALHNHYTQKSLSLSPGK |
| 52 | Sp34 | QDGNEEMGG |
| | epitope | |

### Protease-Cleavable Linkers

The protease-cleavable linker comprises a peptide substrate cleavable by a protease linking the shielding domain to the antibody heavy or light chains. The protease-cleavable linker comprises one or more protease substrate sequence and optional linker spacer sequences (see FIGS. 5-7). In some embodiments, the shielding sequences exist as pairs of sequences that can be fused to the heavy chain and light chain. For example, for each of the two Fab arm domains of a bispecific antibody, a shielding sequence is fused to the N-terminus of the antibody heavy chain via one protease-cleavable linker and the complement sequence is fused to the N-terminus of the antibody light chain via another protease-cleavable linker.

Many disease tissues, including tumor microenvironment and inflammation site, are abundant with various types of proteases whose overexpression correlate with the disease progression. In disease tissues, the protease-cleavable linker sequences of the shielded antibody are recognized by appropriate type of protease and the shields can be released from the antibody chains. For example, in one binding arm of a shielded bispecific antibody, the protease may cleave both of the two protease-cleavable linkers or one of the two protease-cleavable linker sequences, so the shielding domain is inactive. In either case, the shielding domain would not be able to interfere or block the binding of the Fab arm to its target antigen. As a result, the shielded antibody is converted into an active antibody to bind and exert its functional activity to its target (FIG. 7).

In some embodiments, the protease-cleavable linker sequences linking the two shielding domains to the two Fab domains in a shielded antibody comprise the same sequences to be cleaved by the same type of protease.

In some embodiments, the protease-cleavable linker sequences linking the two masking domains and the two Fab domains in a shielded antibody comprise different sequences with substrate sequences cleaved by different types of proteases.

Among the family of matrix metalloproteinases (MMPs), MMP2 and MMP9 are up regulated in many types of cancers, including breast, colorectal and lung cancers. Besides, the expression and activity of MMP2 and MMP9 also correlates to the progression of many autoimmune disorders and inflammatory diseases, including rheumatoid arthritis, psoriasis, multiple sclerosis, chronic obstructed pulmonary disease, inflammatory bowel disease and osteoporosis (Lin, Lu et al. 2020). The disclosure provides for the protease-cleavable linker sequence comprising substrate peptide sequence cleaved by MMP2 and MMP9. As non-limiting examples, the disclosure provides for the MMP2 and MMP9 cleavable substrate peptide sequences set forth as SEQ ID NOs: 53-57. As non-limiting examples, the disclosure provides for the MMP3 cleavable substrate peptide sequences set forth as SEQ ID NOs: 58.

The urokinase plasminogen activator (uPA) has been reported to be overexpressed in many types of cancer, especially the breast cancer (Banys-Paluchowski, Witzel et al. 2019). uPA is a serine protease that can catalyze the conversion of plasminogen to plasmin which can degrade the basement membrane or extracellular matrix. The matrix degradation can facilitate tumor cells migration and invasion into the surrounding tissue. The disclosure provides for the protease-cleavable linker sequence comprising substrate peptide sequence cleaved by uPA. As non-limiting examples, the disclosure provides for the uPA-cleavable substrate peptide sequence set forth as SEQ ID NOs: 59 and 60 in Table 6.

TABLE 6

| SEQ | | |
|---|---|---|
| ID | | Amino Acid |
| NO: | Description | Sequence |
| 53 | Substrate sequence for MMP2 | GPLGVR |
| | and MMP9 | |
| 54 | Substrate sequence for MMP2 | PLGLAR |
| | and MMP9 | |
| 55 | Substrate sequence for MMP2 | PLGLAG |
| | and MMP9 | |
| 56 | Substrate sequence for MMP2 | IPVSLRSG |
| | and MMP9 | |
| 57 | Substrate sequence for MMP2 | GPLGMLSQ |
| | and MMP9 | |
| 58 | Substrate sequence for MMP3 | RPKPVEVWRK |
| 59 | Substrate sequence for uPA | LSGRSDNH |
| 60 | Substrate sequence for uPA | TGRGPSWV |

The protease-cleavable linker of the present disclosure can include one or more linker peptides interposed between, e.g., shielding sequence and protease substrate peptide sequence, and/or between protease substrate peptide sequence and antibody Fabs.

Suitable linkers (also referred to as “spacers”) can be readily selected and can be of any of a number of suitable lengths, such as from 1 amino acid to 30 amino acids (e.g., any specific integer between 1 and 30, or from 1 amino acid (e.g., Gly) to about 20 amino acids, from 2-15, 3-12, 4-10, 5-9, 6-8, or 7-8 amino acids).

Exemplary linkers include glycine polymers (G)ₙ, glycine-serine polymers (including, for example, (GS)ₙ, (GSGGS)ₙ and (GGGS)ₙ, where n is an integer of at least one, e.g., 1, 2, 3, 4, 5, 6, 7, 8, 9, 10, 11, 12, 13, 14, 15, 16, 17, 18, 19, 20), glycine-alanine polymers, alanine-serine polymers, alanine-proline, immunoglobulin isotype and subtype hinge that can comprise IgG₁, IgG₂, IgG₃, IgG₄, IgA, IgE, IgM, and other flexible linkers known in the art. Both Gly and Ser are unstructured, and therefore can serve as a neutral tether between components.

In certain embodiments, the linker is a Glycine polymer. Glycine accesses significantly more phi-psi space than even alanine and is much less restricted than residues with longer side chains (Scheraga 2008). Exemplary linkers can comprise amino acid sequences including, but not limited to: GGS; GGSG; GGSGG; GGGGS; GSGSG; GSGGG; GGGSG; GSSSG, and the like.

In certain embodiments, the linker is an Alanine-Proline polymer. Exemplary linkers can comprise amino acid sequences including, but not limited to (AP)ₙ, where n is an integer of at least one, e.g., 1, 2, 3, 4, 5, 6, 7, 8, 9, 10, 11, 12, 13, 14, 15, 16, 17, 18, 19, 20).

In certain embodiments, the linker is a rigid linker (Chen, Zaro et al. 2013). Exemplary rigid linkers can comprise amino acid sequences including, but not limited to, proline-rich sequence, (XP)ₙ, with X designating any amino acid, preferably Ala, Lys, or Glu, where n is an integer of at least one, e.g., 1, 2, 3, 4, 5, 6, 7, 8, 9, 10, 11, 12, 13, 14, 15, 16, 17, 18, 19, 20). Exemplary rigid linkers can also comprise amino acid sequences including, but not limited to, alpha helix-forming linkers with the sequence of (EAAAK)ₙ, where n is an integer of at least one, e.g., 1, 2, 3, 4, 5, 6, 7, 8, 9, 10, 11, 12, 13, 14, 15, 16, 17, 18, 19, 20).

### Expression and Purification of Antibodies

The antibodies of the disclosure can be encoded by one or more nucleic acids for protein expression. For example, a shielded CD3×CD38 bispecific antibody of the present disclosure can be encoded by a single nucleic acid (e.g., a single nucleic acid comprising nucleotide sequences that encode the light and heavy chain polypeptides of the shielded antibody), or by two or more separate nucleic acids, each of which encodes a different part of the shielded parental antibody.

The appropriate recombinant DNA is prepared by the DNA recombination techniques and then transfected into mammalian cells, the corresponding anti-CD3 and anti-CD38 antibodies are expressed, purified, identified, and/or screened.

A bispecific antibody can be generated from anti-CD3 and anti-CD38 antibodies using the controlled Fab arm exchange or other bispecific antibody generation process to produce a bispecific antibody which shows biological effects of simultaneous binding to CD38 and CD3. Bispecific antibody affinity and blocking efficiency are identified through in vitro experiments.

The nucleic acids described herein can be inserted into vectors, e.g., nucleic acid expression vectors and/or targeting vectors. Such vectors can be used in various ways, e.g., for the expression of a pro-antibody (shielded antibody) with a masking domain described herein in a cell or transgenic animal. Vectors are typically selected to be functional in the host cell in which the vector will be used. A nucleic acid molecule encoding an antibody, e.g., a pro-antibody with a masking domain described herein, may be amplified/expressed in prokaryotic, yeast, insect (baculovirus systems) and/or eukaryotic host cells. Selection of the host cell will depend in part on whether the antibodies disclosed herein, such as a shielded CD3×CD38 bispecific antibody described herein, is to be post-translationally modified (e.g., glycosylated and/or phosphorylated). If so, yeast, insect, or mammalian host cells are preferable. Expression vectors typically contain one or more of the following components: a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element.

In some embodiments, a leader or signal sequence is engineered at the N-terminus of the antibodies, e.g., a shielded CD3×CD38 bispecific antibody, described herein to guide its secretion. The secretion of the shielded CD3×CD38 bispecific antibody from a host cell will result in the removal of the signal peptide from the antibody. Thus, the mature shielded CD3×CD38 bispecific antibody will lack any leader or signal sequence. In some embodiments, such as where glycosylation is desired in a eukaryotic host cell expression system, one may manipulate the various presequences to improve glycosylation or yield. For example, one may alter the peptidase cleavage site of a signal peptide, or add presequences, which also may affect glycosylation.

The disclosure further provides a cell (e.g., an isolated or purified cell) comprising a nucleic acid or vector of the disclosure. The cell can be any type of cell capable of being transformed with the nucleic acid or vector of the disclosure so as to produce a polypeptide encoded thereby. For example, to express a shielded CD3×CD38 bispecific antibody described herein, DNAs encoding partial or full-length light and heavy chains, obtained as described above, are inserted into expression vectors such that the genes are operatively linked to transcriptional and translational control sequences.

Methods of introducing nucleic acids and vectors into isolated cells and the culture and selection of transformed host cells in vitro are known in the art and include the use of calcium chloride-mediated transformation, transduction, conjugation, triparental mating, DEAE, dextran-mediated transfection, infection, membrane fusion with liposomes, high velocity bombardment with DNA-coated microprojectiles, direct microinjection into single cells, and electroporation.

After introducing the nucleic acid or vector of the disclosure into a host cell, the cell is cultured under conditions suitable for expression of the encoded sequence. The antibody, antigen binding fragment, or portion of the antibody then can be isolated from the cell.

In certain embodiments, two or more vectors that together encode the shielded CD3×CD38 bispecific antibody described herein, can be introduced into a host cell.

Purification of an antibody, e.g., a shielded CD3×CD38 bispecific antibody, described herein, which is secreted into the cell media, can be accomplished using a variety of techniques including affinity, immunoaffinity or ion exchange chromatography, molecular sieve chromatography, preparative gel electrophoresis or isoelectric focusing, chromatofocusing, and high-pressure liquid chromatography. For example, antibodies comprising a F_{c} region may be purified by affinity chromatography with Protein A, which selectively binds the F_{c} region. Modified forms of the antibodies, such as a shielded CD3×CD38 bispecific antibody, may be prepared with affinity tags, such as hexahistidine or other small peptide such as FLAG (Eastman Kodak Co., New Haven, Conn.) or Myc (Invitrogen) at either its carboxyl or amino terminus and purified by a one-step affinity column. For example, Poly histidine binds with great affinity and specificity to nickel, thus an affinity column of nickel (such as the Qiagen® nickel columns) can be used for purification of Poly histidine-tagged selective binding agents. In some instances, more than one purification step may be employed.

### Effects of Shielded CD3×CD38 Bispecific Antibodies on Binding and Functional Activity

The shielded CD3×CD38 bispecific antibody disclosed herein can inhibit or block the capability of the Fab arm to bind to the respective antigens, CD3 and CD38. The masking domains may reduce the maximum binding capacity of the shielded bispecific antibody in binding to the respective antigens. The masking domains may also reduce the binding affinity of the shielded bispecific antibody in binding to the respective antigens.

When the masking domains are cleaved off by protease, the shielded antibody is converted to an active bispecific antibody with the restoration of the capability of the antibody in binding to its antigen. The removal of masking domains from the shielded bispecific antibody can be realized by in vitro protease cutting assay using recombinant or purified protease. The removal of the masking domains from the shielded bispecific antibody can also be realized in vivo by proteases overexpressed in disease site. The removal of the masking domains can be assessed by comparing the molecular weight of heavy chain and light chain of shielded antibodies with the masking domains to the active antibody without the masking domains by SDS-PAGE, IEX, or HIC analyses.

In vitro and cell-based assays are well described in the art for use in determining the pro-antibody (shielded bispecific antibody), active antibody, and converted antibody after protease cleavage in binding to its antigen. For example, the binding of antibody may be determined by ELISA by immobilizing a recombinant or purified antigen, sequestering antibody with the immobilized antigen and determining the amount of bound antibody. This can also be performed using a Biacore® instrument for kinetic analysis of binding interactions. For cell-based binding assay, the binding of an antibody may be determined by flow cytometry by incubating the antibody with cells expressing antigens on cell surface and determining the amount of antibody bound to cell surface antigen.

### Pharmaceutical Composition

The antibodies such as shielded CD3×CD38 bispecific antibodies for use according to the present disclosure can be formulated in compositions, especially pharmaceutical compositions, for use in the methods herein. Such compositions comprise a therapeutically or prophylactically effective amount of an antibody, e.g., a bispecific antibody, described in this disclosure and a suitable carrier, e.g., a pharmaceutically acceptable agent. Typically, the antibody described in this disclosure is sufficiently purified for administration to an animal before formulation in a pharmaceutical composition.

In some embodiment, the present disclosure provides a composition comprising an anti-CD38 antibody or antigen binding portion thereof. In some embodiment, the present disclosure provides a pharmaceutical composition comprising an anti-CD38 antibody or antigen binding portion thereof and a pharmaceutically acceptable carrier.

In some embodiment, the present disclosure provides a composition comprising an anti-CD3 antibody or antigen binding portion thereof. In some embodiment, the present disclosure provides a pharmaceutical composition comprising an anti-CD3 antibody or antigen binding portion thereof and a pharmaceutically acceptable carrier.

In some embodiment, the present disclosure provides a composition comprising a shielded or non-shielded CD3×CD38 bispecific antibody. In some embodiment, the present disclosure provides a pharmaceutical composition comprising a shielded or non-shielded CD3×CD38 bispecific antibody and a pharmaceutically acceptable carrier.

Pharmaceutically acceptable agents include carriers, excipients, diluents, antioxidants, preservatives, coloring, flavoring, and diluting agents, emulsifying agents, suspending agents, solvents, fillers, bulking agents, buffers, delivery vehicles, tonicity agents, cosolvents, wetting agents, complexing agents, buffering agents, antimicrobials, and surfactants.

The composition can be in liquid form or in a lyophilized or freeze-dried form and may include one or more lyoprotectants, excipients, surfactants, high molecular weight structural additives and/or bulking agents.

In some embodiments, the composition comprises a shielded CD3×CD38 bispecific antibody, or an anti-CD38 antibody or an antigen-binding portion thereof, or an anti-CD3 antibody or an antigen binding portion thereof, and at least one buffer, at least one stabilizer, and/or at least one surfactant.

In some embodiments, the composition disclosed herein is liquid. In some embodiments, the composition is formulated for subcutaneous injection. In some embodiments, the composition is sterile. In some embodiments, the composition further comprises histidine HCl, trehalose, methionine and/or polysorbate.

Compositions can be suitable for parenteral administration. Exemplary compositions are suitable for injection or infusion into an animal by any route available to the skilled worker, such as intraarticular, subcutaneous, intravenous, intramuscular, intraperitoneal, intracerebral (intraparenchymal), intracerebroventricular, intramuscular, intraocular, intraarterial, intralesional, intrarectal, transdermal, oral, and inhaled routes.

Pharmaceutical compositions described herein can be formulated for controlled or sustained delivery in a manner that provides local concentration of the product (e.g., bolus, depot effect) sustained release and/or increased stability or half-life in a particular local environment.

In some embodiments, the CD3×CD38 bispecific antibody or an anti-CD38 antibody or an antigen-binding portion thereof, or an anti-CD3 antibody or an antigen binding portion thereof, can be present in a pharmaceutical composition at a concentration of 1 mg/mL to 250 mg/mL, 10 mg/mL to 250 mg/mL, 1 mg/mL to 100 mg/mL, 2 mg/mL to 50 mg/mL, and 2 mg/mL to 40 mg/mL.

### Methods of Treatment and Use

In some embodiments, the present disclosure provides methods for treating or preventing a disease or disorder in a subject in need thereof comprising administering an effective amount of a shielded CD3×CD38 bispecific antibody or an anti-CD38 antibody or an antigen-binding portion thereof disclosed herein to the subject. The diseases or disorder is selected from CD38-mediated diseases or disorders, e.g., human cancers, including gastric and colorectal cancer, pancreatic cancer, prostate cancer, lung cancer, hepatocellular cancer, triple-negative breast cancer, nasopharyngeal cancer-cervical cancer, hematologic malignancies such as MM, lymphoma, acute myeloid leukemia, chronic lymphocytic leukemia, acute B lymphoblastic leukemia, and others, heart diseases, viral infection including HIV infection, asthma and other respiratory inflammatory disorders, allergic airway disease, fetomaternal tolerance, autism spectrum disorders, glomerular sclerosis, inflammatory bowel disease, rheumatoid arthritis, diabetes mellitus, diabetes, chronic autoimmune thyroiditis and Graves' disease, and age-related neurodegenerative or neuro-inflammatory diseases, such as Alzheimer's Disease.

Some embodiments also provide an application of the CD3×CD38 bispecific antibody or an anti-CD38 antibody or an antigen-binding portion thereof in the manufacture of a medicament for the treatment or prevention of cancer, preferably, lung cancer, breast cancer, colorectal cancer, gastric cancer, intestinal cancer, and/or pancreatic cancer.

In some embodiments, the present disclosure provides a shielded CD3×CD38 bispecific antibody or an anti-CD38 antibody disclosed or an antigen-binding portion thereof herein for use in treating or preventing a disease or disorder selected from CD38-mediated diseases or disorders.

In some embodiments, the present disclosure provides the use of a shielded CD3×CD38 bispecific antibody or an anti-CD38 antibody or an antigen-binding portion thereof disclosed herein for the manufacture of a medicament for use in treating or preventing a disease or disorder selected from CD38-mediated diseases or disorders.

In some embodiment, the present disclosure provides a shielded CD3×CD38 bispecific antibody or an anti-CD38 antibody or an antigen-binding portion thereof described herein for use in treating or preventing gastric, lung, pancreatic, colorectal, and other cancers. In contrast to corresponding therapeutic antibodies, the shielded CD3×CD38 bispecific antibody may have comparable efficacy in treating these diseases due to the conversion of the shielded antibody to active antibody specifically in disease sites by the removal of the shielding domains by proteases overexpressed in disease sites. However, the shielded antibody may have reduced systematic toxicity due to the masking of the antibody activity by the shielding domains in normal tissues that lack sufficient amounts of proteases needed to cleave off the masking domains. The shielded bispecific antibody described herein may be efficacious as the corresponding therapeutic antibody in treating diseases but with much improved safety profile. Due to the improved safety profile, increased levels of dosing comprising the shielded bispecific antibodies may be administered to patients with improved treatment efficacy.

In some embodiments, the disclosure also provides for a method of treating cancer in a subject, comprising administering a therapeutically effective amount of a shielded CD3×CD38 bispecific antibody. The disclosure also provides for use of the shielded bispecific provided herein in a method of treating cancer; and for use of the shielded CD3×CD38 bispecific antibodies provided herein in the manufacture of a medicament for use in cancer. Exemplary cancers include, but are not limited to MM, non-small cell lung cancer, acute myeloid leukemia, female breast cancer, pancreatic cancer, colorectal cancer, and peritoneum cancer.

In some embodiments, a bispecific antibody or an anti-CD38 antibody or an antigen-binding portion thereof disclosed herein can effectively inhibit CD38 receptor association with integrins and/or downstream signaling.

In some embodiments, a bispecific antibody or an anti-CD38 antibody or an antigen-binding portion thereof disclosed herein can be used a combination with chemotherapy. For example, a combination regimen for treating cancer can use higher doses of chemotherapy and a CD3×CD38 bispecific antibody to determine the best synergistic partners.

In some embodiments, the present disclosure provides a conjugate of a bispecific antibody or an anti-CD38 antibody or an antigen-binding portion thereof disclosed herein and another therapeutic agent. In some embodiments, the other therapeutic agent is a cytotoxin or a radioactive isotope.

All combinations of the various elements described herein are within the scope of the disclosure unless otherwise indicated herein or otherwise clearly contradicted by context.

This disclosure will be better understood from the following Experimental Details. However, one skilled in the art will readily appreciate that the specific methods and results discussed are merely illustrative of the disclosure as described more fully in the claims that follow thereafter.

### Example 1: Expression and Purification of Anti-CD38 and Anti-CD3 Antibodies

The masked and unmasked anti-CD3 and anti-CD38 antibody genes were put in separate plasmids to evaluate the preparation of monoclonal anti-CD3 and anti-CD38 antibodies and for preparation of anti-CD3 and anti-CD38 bispecific antibodies. In the case of shielded antibodies, heavy chain and light chain constructs expressing anti-CD3 and anti-CD38 shielded mAbs were prepared. Plasmids encoding heavy chains and light chains of these anti-CD3 and anti-CD38 masked antibodies were co-transfected into Expi293F cells following the transfection kit instructions (Thermo Scientific). Cells were spun down five days post transfection, and the supernatant are passed through a 0.2 μm filter. The purifications of expressed masked antibody supernatants were conducted by affinity chromatography over protein A agarose columns (GE Healthcare Life Sciences). The purified masked antibodies were buffer exchanged into DPBS, pH7.2 by dialysis, and protein concentrations were determined by UV absorbance at 280 nm. Unshielded anti-CD3 and anti-CD38 monoclonal antibodies were prepared and purified in a similar way.

The anti-CD3 and anti-CD38 shielded mAbs had masking domain fused to the N-terminus of antibody heavy chains and light chains with the substrate sequences for MMP2/MMP9. The purified masked antibodies were characterized by SDS-PAGE analysis.

### Example 2: Protease Digestion of Antibodies with Masking Domains

In vitro protease cutting assays were set up to evaluate whether the shielding domains were removed from the masked Abs by proteases. For MMP2, recombinant human MMP2 was activated by incubating with p-amino phenylmercuric acetate (APMA) according to manufacturer's instruction (R&D Systems). Ten mg of masked Abs were incubated with 50 ng of activated MMP2 overnight at 37° C. The digestions of the masked mAbs were evaluated by SDS-PAGE under reduced condition. The molecular weight of the heavy chain and light chain from the digested masked Abs were slightly smaller relative to the corresponding undigested pro-antibodies. Upon protease treatment, the molecular weights of the capped mAbs were closer to that of the reference mAb.

### Example 3: Anti-CD3 Antibodies Bind to Recombinant Human and Cynomolgus Monkey CD3 Antigen in ELISA Assays

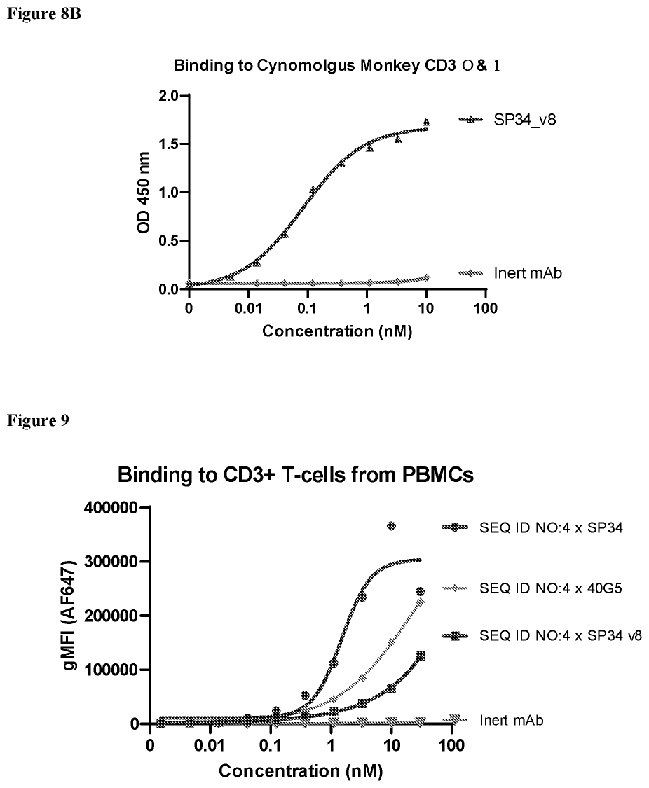

Biotin labeled human recombinant CD3 protein, consisting of CD3 epsilon and CD3 delta chains, was bound to a streptavidin coated ELISA plate to capture the antigen on the solid phase. Varying concentrations of the tested anti-CD3 antibodies were then added to the coated plates and the bound antibodies were detected and quantified using standard ELISA protocols. The results, shown in FIG. 8A, demonstrate the ability of these anti-CD3 antibodies to bind to the intended human CD3 target. The antibodies display a range of affinities for the antigen in this assay format. This range of affinities is valuable since different CD3 affinities may be ideal in disease specific applications or in specific pairings with other antibodies in a bispecific format.

Biotin labeled cynomolgus monkey recombinant CD3 protein, consisting of CD3 epsilon and CD3 delta chains, was bound to a streptavidin coated ELISA plate to capture the antigen on the solid phase. Varying concentrations of the tested anti-CD3 antibody was then added to the coated plates and the bound antibodies were detected and quantified using standard ELISA protocols. The results, shown in FIG. 8B, demonstrated the ability of this anti-CD3 species to bind and cross react with cynomolgus monkey CD3 protein in addition to their demonstrated binding to the human CD3 protein. The ability to also bind the cynomolgus monkey CD3 protein is an important enabling property for drug development since it can facilitate the use of these animals for toxicology and other needed studies for these antibodies and especially for bispecific antibodies that contain them.

Bispecific antibodies consisting of a single anti-CD38 arm paired with a variety of anti-CD3 arms were mixed in a series of increasing bispecific concentrations with human PBMCs containing CD3 positive T cells. The binding of the bispecific antibodies was detected and quantified using standard flow cytometric methods. The results are shown in FIG. 9. These data demonstrate that the tested anti-CD3 antibodies are capable of binding to the human CD3 protein as it is natively presented on the surface of the intended target T cells in addition to their demonstrated ability to bind to the isolated recombinant protein. Hence the CD3 antibodies demonstrated both ELISA and cell binding.

### Example 4: Anti-CD38 Antibodies Bind to Recombinant Human, Recombinant Cynomolgus Monkey, and Recombinant Mouse CD38 Antigen in ELISA Assays

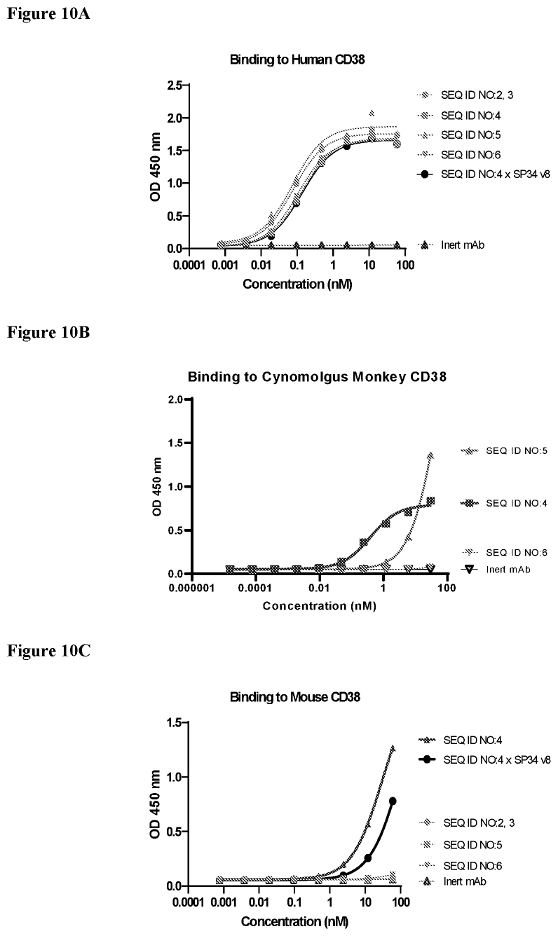

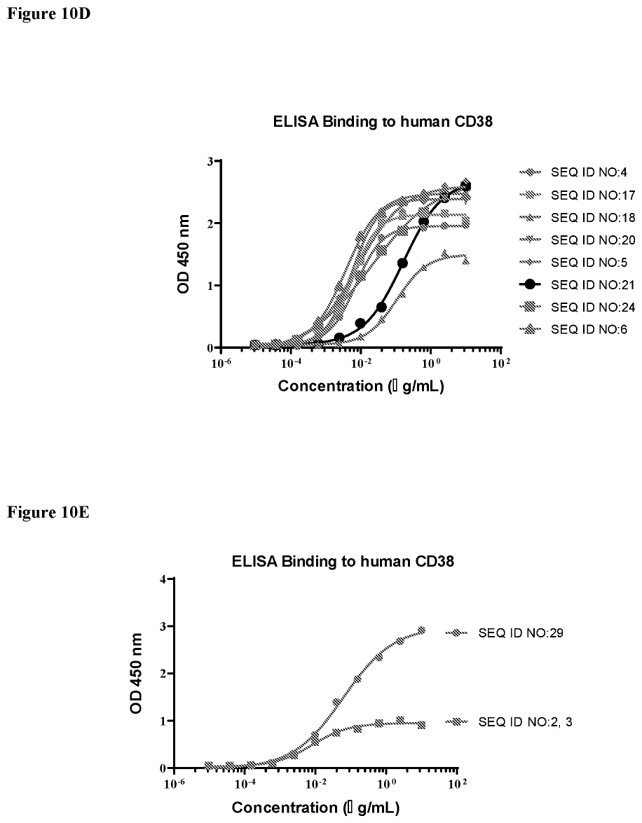

Biotin labeled recombinant CD38 protein was bound to streptavidin coated ELISA plates to capture the antigen on the solid phase. Varying concentrations of the tested anti-CD38 antibodies were then added to the coated plates and the bound antibodies were detected and quantified using standard ELISA protocols. The results, shown in FIG. 10 demonstrated the ability of these anti-CD38 species to bind to the intended human CD38 target and in some cases also to the mouse antigen. Daratumumab is a known and currently marketed CD38 specific antibody with clinical activity (heavy chain variable region and light chain variable regions with SEQ ID NO: 2, 3) and single domain antibody variable heavy chains with SEQ ID NO: 5 and 6 were included as positive controls and for comparative purposes. The results, shown in FIG. 10, demonstrated the ability of these novel anti-CD38 species SEQ ID NO: 4, 17, 18, 20, 5, 21, 24, 6, 29 to bind to the intended human CD38 targets. The antibodies displayed a range of affinities for the antigen in this assay format. The SEQ ID NO: 4 anti-CD38 antibody could bind to cynomolgus monkey CD38 antigen in an ELISA format (FIG. 10B). The SEQ ID NO: 4 anti-CD38 antibody could bind to mouse CD38 antigen in an ELISA format (FIG. 10C). Biotin labeled cynomolgus recombinant CD38 protein was bound to a streptavidin coated ELISA plate to capture the antigen on the solid phase. Varying concentrations of the tested anti-CD38 antibodies were then added to the coated plates and the bound antibodies were detected and quantified using standard ELISA protocols. The results, shown in FIG. 10, demonstrated the ability of these novel anti-CD38 species to bind and cross react with the cynomolgus monkey CD38 target in addition to their demonstrated binding to the human CD38 protein. The ability to also bind the cynomolgus monkey CD38 protein is an important enabling property for drug development since it can facilitate the use of these animals for toxicology and other needed studies for these antibodies and especially for bispecific antibodies that contain them.

ELISA-based binding assay is employed to evaluate the binding to CD3 and CD38 by a CD3×CD38 bispecific antibody. In this assay, human CD38 is coated on the plate and then the CD3×CD38 bispecific antibody or a mixture of CD3 antibody and CD38 antibody, along with recombinant CD3 with a His tag are added. After washing off the non-specific binding, the presence of CD3 is detected by an HRP-conjugated anti-his secondary antibody (BioLegend). The CD3×CD38 bispecific antibody, but not by the mixture of the two parental antibodies, dose-dependently recruits CD3. Hence, the bispecific antibody is capable of binding CD3 and CD38 simultaneously.

### Example 5: Anti-CD38×CD3 Antibodies Bind to and Activate a Human CD3 Positive T Cell Line in the Presence of H929, a CD38 Positive Target Cell Line

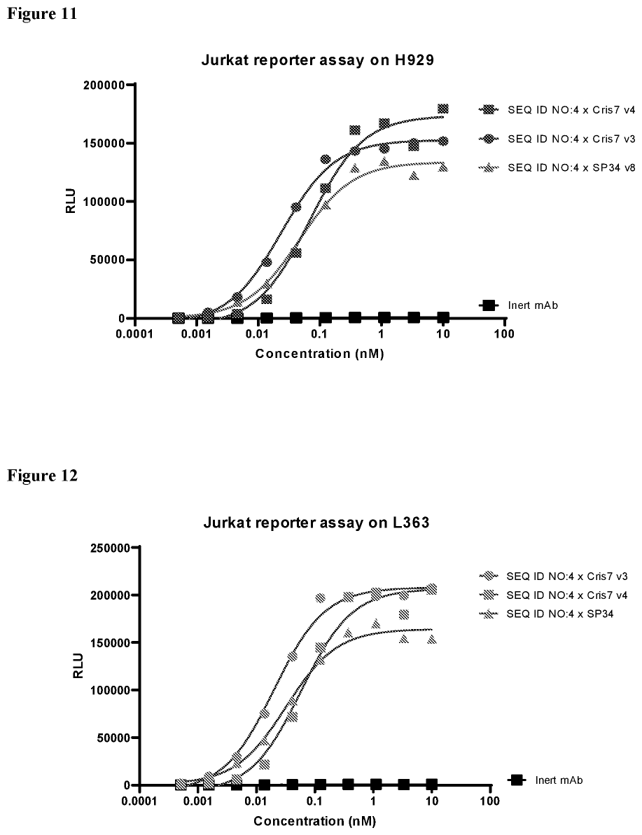

Bispecific antibodies comprising a single anti-CD38 arm paired with a variety of anti-CD3 arms, respectively, were mixed in a series of increasing concentrations of the bispecific antibodies with 2 cell lines: one a reporter T cell line and the other a target cell line H929, a CD38 positive multiple myeloma cell line. The reporter T cell line was the human CD3 positive Jurkat T cell line engineered to produce an easily measured signal when the T cell receptors on their surface were bound and activated for killing. The target cell line was the H929 human CD38 positive multiple myeloma cell line. In cancer therapy, a CD38×CD3 bispecific antibody was expected to bind to the CD3 on an attacking killer T cell and to CD38 on a target cancer cell. The results shown in FIG. 11 demonstrated concentration dependent activation of the T cells as a consequence of the binding of the bispecific antibodies to both CD3 and CD38 proteins. These data demonstrated that the tested anti-CD3 and anti-CD38 arms were capable of binding to both proteins as they were presented on the cell surface of cells. There was minimal activation in the presence of the inert arm in the bispecific antibody. The data further supported the conclusion that the binding also mediated the desired activation of the T cell pathways necessary for target cancer cell killing.

### Example 6: Anti-CD38×CD3 Antibodies Bind to and Activate a Human CD3 Positive T Cell Line in the Presence of L363, a CD38 Positive Target Cell Line

Bispecific antibodies comprising a single anti-CD38 arm paired with a variety of anti-CD3 arms, respectively, were mixed in a series of increasing concentrations of the bispecific antibodies with 2 cell lines: one a reporter T cell line and the other a target cell line. The reporter T cell line was the human CD3 positive Jurkat T cell line engineered to produce an easily measured signal when the T cell receptors on their surface were bound and activated for killing. The target cell line was the L363 human CD38 positive multiple myeloma cell line. In cancer therapy, a CD38×CD3 bispecific antibody was expected to bind to the CD3 on an attacking killer T cell and to CD38 on a target cancer cell. The results shown in FIG. 12 demonstrated concentration dependent activation of the T cells as a consequence of the binding of the bispecific antibodies to both CD3 and CD38 proteins. These data demonstrated that the tested anti-CD3 and anti-CD38 arms were capable of binding to both proteins as they were presented on the cell surface of cells. There was minimal activation in the presence of the inert arm in the bispecific antibody. The data further supported the conclusion that binding also mediated the desired activation of the T cell pathways necessary for target cancer cell killing.

### Example 7: Anti-CD38×CD3 Antibodies Bind to and Activate a Human CD3 Positive T Cell Line in the Presence of RPMI 8226, a CD38 Positive Target Cell Line

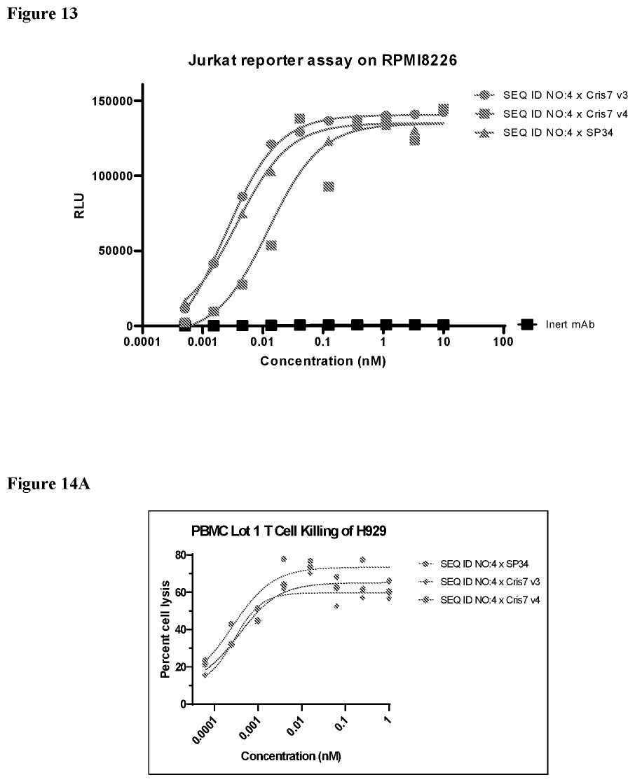

Bispecific antibodies comprising a single anti-CD38 arm paired with a variety of anti-CD3 arms, respectively, were mixed in a series of increasing concentrations of the bispecific antibodies with 2 cell lines: one a reporter T cell line and the other a target cell line. The reporter T cell line was the human CD3 positive Jurkat T cell line engineered to produce an easily measured signal when the T cell receptors on their surface were bound and activated for killing. The target cell line was the RPMI 8226 human CD38 positive multiple myeloma cell line. In cancer therapy, a CD38×CD3 bispecific antibody was expected to bind to the CD3 on an attacking killer T cell and to CD38 on a target cancer cell. The results shown in FIG. 13 demonstrated concentration dependent activation of the T cells as a consequence of the binding of the bispecific antibodies to both CD3 and CD38 proteins. These data demonstrated that the tested anti-CD3 and anti-CD38 arms were capable of binding to both proteins as they were presented on the cell surface of cells. There was minimal activation in the presence of the inert arm in the bispecific antibody. The data further supported the conclusion that binding also mediated the desired activation of the T cell pathways necessary for target cancer cell killing.

### Example 8: Anti-CD38×CD3 Antibodies had Directed PBMC Killing of H929, a CD38 Positive Target Cell Line

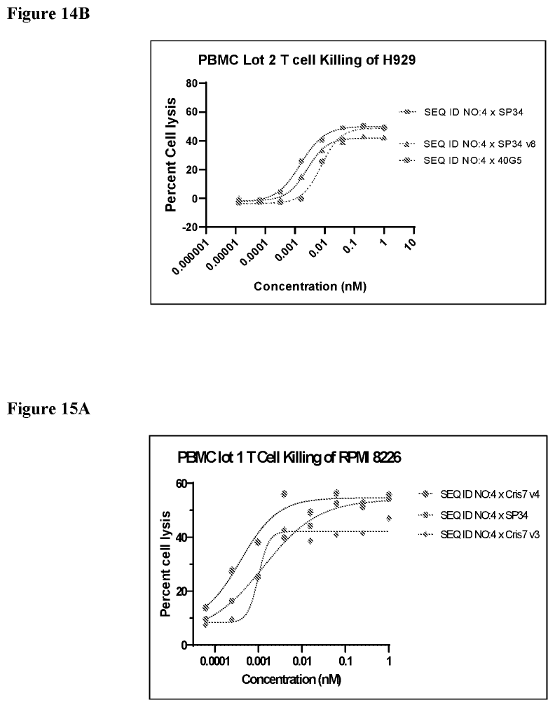

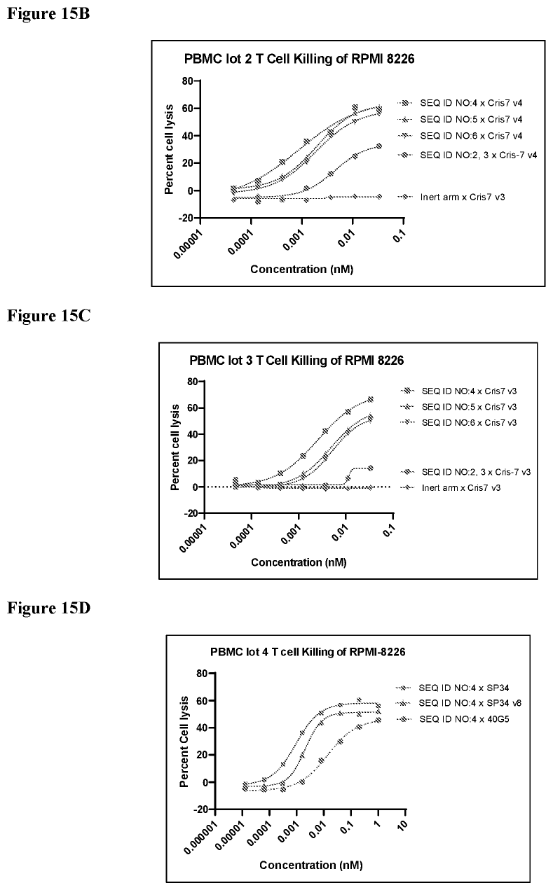

Bispecific antibodies comprising a single anti-CD38 arm paired with a variety of anti-CD3 arms, respectively, were mixed in a series of increasing concentrations of the bispecific antibodies with PBMC lots and a target cell line. The target cell line was the H929 human CD38 positive multiple myeloma cell line. The results shown in FIG. 14 demonstrated concentration dependent T cell killing as a consequence of the binding of the bispecific antibodies to both CD3 and CD38 proteins. These data demonstrated that the tested anti-CD3 and anti-CD38 arms were capable of binding to both proteins as they were presented on the cell surface of cells in addition to their demonstrated ability to bind to the isolated recombinant proteins. The data further supported the conclusion that the binding also mediated the desired T cell directed target cancer cell killing. These experiments with two different PBMC donor T cells, that were used as effector cells, led to potent PBMC donor T cell killing of H929 cells. The data further supported the conclusion that the binding to both CD3 and CD38 proteins also resulted in the bispecific antibody mediated activation of human primary T cells and their killing of the target cancer cells.

### Example 9: Anti-CD38×CD3 Antibodies had Directed PBMC Killing of RPMI 8226, a CD38 Positive Target Cell Line

Bispecific antibodies comprising a single anti-CD38 arm paired with a variety of anti-CD3 arms, respectively, were mixed in a series of increasing concentrations of the bispecific antibodies with PBMC lots and a target cell line. The target cell line was the RPMI 8226 human CD38 positive multiple myeloma cell line. The results shown in FIG. 15 demonstrated concentration dependent T cell killing as a consequence of the binding of the bispecific antibodies to both CD3 and CD38 proteins. These data demonstrated that the tested anti-CD3 and anti-CD38 arms were capable of binding to both proteins as they were presented on the cell surface of cells in addition to their demonstrated ability to bind to the isolated recombinant proteins. The data further supported the conclusion that the binding to both CD3 and CD38 proteins also mediated the desired T cell directed target cancer cell killing. These experiments, with 4 different PBMC donor T cells, that were used as effector cells, led to potent PBMC donor T cell killing of RPMI 8226 cells. The data further supported the conclusion that the binding to both CD3 and CD38 proteins also resulted in the bispecific antibody mediated activation of human primary T cells and their killing of the target cancer cells.

### Example 10: Anti-CD38×CD3 Antibodies had Directed PBMC Killing of L363, a CD38 Positive Target Cell Line

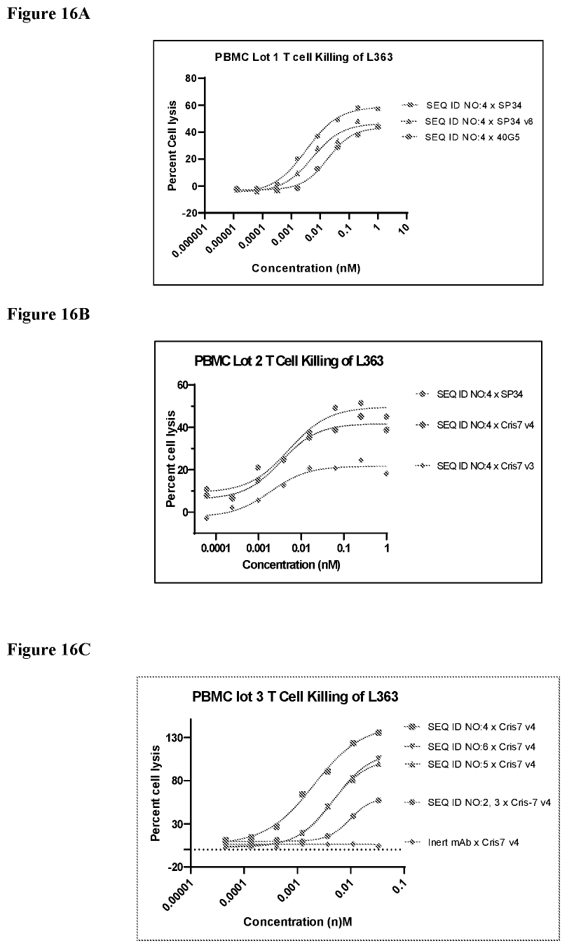

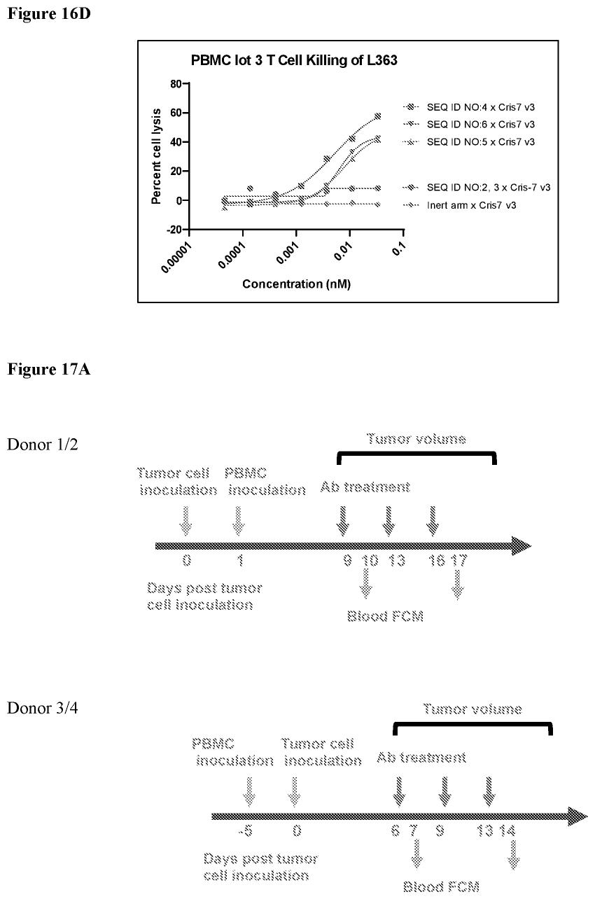

Bispecific antibodies comprising a single anti-CD38 arm paired with a variety of anti-CD3 arms, respectively, were mixed in a series of increasing concentrations of the bispecific antibodies with PBMC lots and a target cell line. The target cell line was the L363 human CD38 positive multiple myeloma cell line. The results shown in FIG. 16 demonstrated concentration dependent T cell killing as a consequence of the binding of the bispecific antibodies to both CD3 and CD38 proteins. These data demonstrated that the tested anti-CD3 and anti-CD38 arms were capable of binding to both proteins as they were presented on the cell surface of cells in addition to their demonstrated ability to bind to the isolated recombinant proteins. The data further supported the conclusion that the binding also mediated the desired T cell directed target cancer cell killing. These experiments, with 3 different PBMC donor T cells, that were used as effector cells, led to potent PBMC donor T cell killing of L363 cells. The data further supported the conclusion that the binding to both CD3 and CD38 proteins also resulted in the bispecific antibody mediated activation of human primary T cells and their killing of the target cancer cells.

### Example 11: In Vivo Anti-Tumor Efficacy of CD3×CD38 Bispecific Antibodies

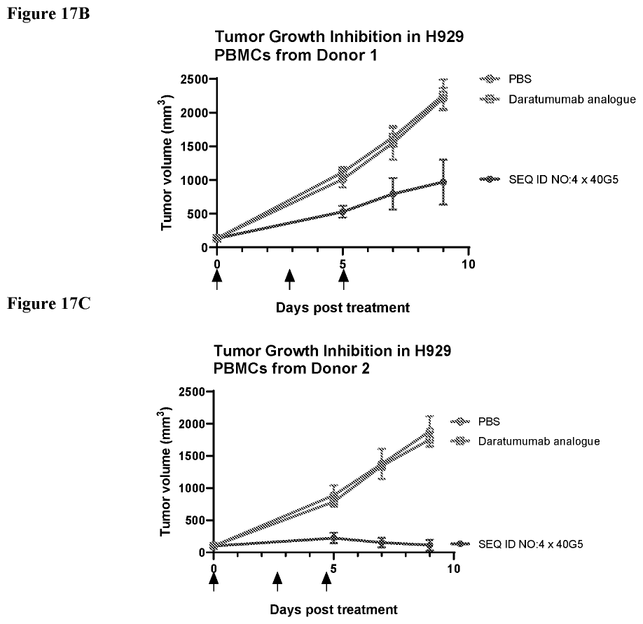

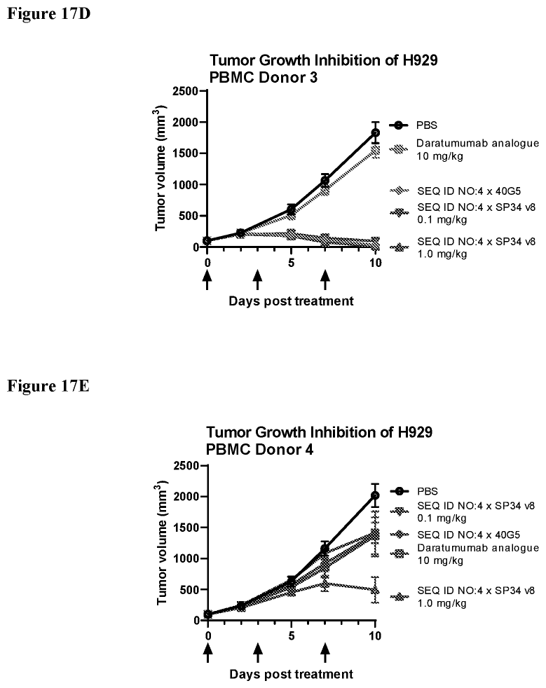

The efficacy of CD3×CD38 bispecific antibodies in tumor cell killing was evaluated in a mouse tumor xenograft model. The novel anti-CD38 antibody as noted in SEQ ID NO: 4 was made as an IgG1 Fc fusion and was paired in a bispecific antibody with a 40G5 CD3 arm known to be capable of directing T cell killing as well as to assess the ability of SEQ ID NO: 4 to direct T cell killing of a CD38 expressing cell. This bispecific antibody was evaluated in an NCI-H929 human multiple myeloma xenograft model in which human PBMCs containing CD3 positive effector T cells were engrafted in immunocompromised mice one day after subcutaneous implant of the cancer cells (FIGS. 17B-17C) or 5 days before subcutaneous implant of the cancer cells (FIGS. 17D-17E). NCI-H929, a CD38-expressing cell line, was inoculated into NCG mice for tumor xenograft establishment. The test molecules was intraperitoneally administered to the mice and the antibody-mediated tumor shrinkage was assessed (FIG. 17A). Because it is understood that the results of these models vary widely depending on the nature of the engrafted T cells from different donors, the experiments were performed using two parallel cohorts of mice, each engrafted with T cells from distinct donors. Two different sets of CD3 arms were used to make the CD38×CD3 bispecific antibody. The control CD3 arm was designated as 40G5 comprising heavy chain variable region SEQ ID NO: 40 and light chain variable region SEQ ID NO:41. The test anti-CD3 arm was designated as SP34 v8 comprising heavy chain variable region SEQ ID NO: 39 and light chain variable region SEQ ID NO:38. The results of the experiment shown in FIG. 17 demonstrated that the CD38×CD3 bispecific antibodies, SEQ ID NO: 4 paired with the 40G5 arm and SEQ ID NO: 4 paired with the SP34 v8 arm, were highly active in inhibiting tumor growth in both cohorts, with partial inhibition of tumor with one T cell donor and complete tumor growth inhibition with the second donor. This inhibition was particularly noteworthy since each cohort was also treated with daratumumab, an anti-CD38 antibody that is approved for clinical use in multiple myeloma and is known to be highly active in multiple myeloma patients. The results showed that daratumumab was inactive in this model, whereas the CD38×CD3 antibody showed clear anti-tumor activity with both donor T cell cohorts. A primary conclusion is that the anti-CD38 arm of the bispecific tested in this experiment is highly efficacious against multiple myeloma in vivo.

### REFERENCES

Akther, S., N. Korshnova, J. Zhong, M. Liang, S. M. Cherepanov, O. Lopatina, Y. K. Komleva, A. B. Salmina, T. Nishimura, A. A. Fakhrul, H. Hirai, I. Kato, Y. Yamamoto, S. Takasawa, H. Okamoto and H. Higashida (2013). “CD38 in the nucleus accumbens and oxytocin are related to paternal behavior in mice.” Mol Brain 6:41.
Allard, B., P. A. Beavis, P. K. Darcy and J. Stagg (2016). “Immunosuppressive activities of adenosine in cancer.” Curr Opin Pharmacol 29:7-16. Amgen. (2021). “Bincyto.”
Antonelli, A., G. Baj, P. Marchetti, P. Fallahi, N. Surico, C. Pupilli, F. Malavasi and E. Ferrannini (2001). “Human anti-CD38 autoantibodies raise intracellular calcium and stimulate insulin release in human pancreatic islets.” Diabetes 50(5): 985-991.
Antonelli, A., P. Fallahi, C. Nesti, C. Pupilli, P. Marchetti, S. Takasawa, H. Okamoto and E. Ferrannini (2001). “Anti-CD38 autoimmunity in patients with chronic autoimmune thyroiditis or Graves' disease.” Clin Exp Immunol 126(3): 426-431.
Antonelli, A. and E. Ferrannini (2004). “CD38 autoimmunity: recent advances and relevance to human diabetes.” J Endocrinol Invest 27(7): 695-707.
Banys-Paluchowski, M., I. Witzel, B. Aktas, P. A. Fasching, A. Hartkopf, W. Janni, S. Kasimir-Bauer, K. Pantel, G. Schon, B. Rack, S. Riethdorf, E. F. Solomayer, T. Fehm and V. Muller (2019). “The prognostic relevance of urokinase-type plasminogen activator (uPA) in the blood of patients with metastatic breast cancer.” Sci Rep 9(1): 2318. Biopharmadive.com. (2021).
Blacher, E., B. Ben Baruch, A. Levy, N. Geva, K. D. Green, S. Garneau-Tsodikova, M. Fridman and R. Stein (2015). “Inhibition of glioma progression by a newly discovered CD38 inhibitor.” Int J Cancer 136(6): 1422-1433.
Blacher, E., T. Dadali, A. Bespalko, V. J. Haupenthal, M. O. Grimm, T. Hartmann, F. E. Lund, R. Stein and A. Levy (2015). “Alzheimer's disease pathology is attenuated in a CD38-deficient mouse model.” Ann Neurol 78(1): 88-103.
Boini, K. M., M. Xia, J. Xiong, C. Li, L. P. Payne and P. L. Li (2012). “Implication of CD38 gene in podocyte epithelial-to-mesenchymal transition and glomerular sclerosis.” J Cell Mol Med 16(8): 1674-1685.
Boison, D. and G. G. Yegutkin (2019). “Adenosine Metabolism: Emerging Concepts for Cancer Therapy.” Cancer Cell 36(6): 582-596.
Bolt, S., E. Routledge, I. Lloyd, L. Chatenoud, H. Pope, S. D. Gorman, M. Clark and H. Waldmann (1993). “The generation of a humanized, non-mitogenic CD3 monoclonal antibody which retains in vitro immunosuppressive properties.” Eur J Immunol 23(2): 403-411.
Boslett, J., C. Hemann, F. L. Christofi and J. L. Zweier (2018). “Characterization of CD38 in the major cell types of the heart: endothelial cells highly express CD38 with activation by hypoxia-reoxygenation triggering NAD(P)H depletion.” Am J Physiol Cell Physiol 314(3): C297-C309.
Brischwein, K., B. Schlereth, B. Guller, C. Steiger, A. Wolf, R. Lutterbuese, S. Offner, M. Locher, T. Urbig, T. Raum, P. Kleindienst, P. Wimberger, R. Kimmig, I. Fichtner, P. Kufer, R. Hofmeister, A. J. da Silva and P. A. Baeuerle (2006). “MT110: a novel bispecific single-chain antibody construct with high efficacy in eradicating established tumors.” Mol Immunol 43(8): 1129-1143.
Bu, X., J. Kato, J. A. Hong, M. J. Merino, D. S. Schrump, F. E. Lund and J. Moss (2018). “CD38 knockout suppresses tumorigenesis in mice and clonogenic growth of human lung cancer cells.” Carcinogenesis 39(2): 242-251.
Burgler, S. (2015). “Role of CD38 Expression in Diagnosis and Pathogenesis of Chronic Lymphocytic Leukemia and Its Potential as Therapeutic Target.” Crit Rev Immunol 35(5): 417-432.
Burgler, S., A. Gimeno, A. Parente-Ribes, D. Wang, A. Os, S. Devereux, P. Jebsen, B. Bogen, G. E. Tjonnfjord and L. A. Munthe (2015). “Chronic lymphocytic leukemia cells express CD38 in response to Th1 cell-derived IFN-gamma by a T-bet-dependent mechanism.” J Immunol 194 (2): 827-835.
Calabretta, E. and C. Carlo-Stella (2020). “The Many Facets of CD38 in Lymphoma: From Tumor-Microenvironment Cell Interactions to Acquired Resistance to Immunotherapy.” Cells 9(4).
Camacho-Pereira, J., M. G. Tarrago, C. C. S. Chini, V. Nin, C. Escande, G. M. Warner, A. S. Puranik, R. A. Schoon, J. M. Reid, A. Galina and E. N. Chini (2016). “CD38 Dictates Age-Related NAD Decline and Mitochondrial Dysfunction through an SIRT3-Dependent Mechanism.” Cell Metab 23(6): 1127-1139.
Cambronne, X. A. and W. L. Kraus (2020). “Location, Location, Location: Compartmentalization of NAD(+) Synthesis and Functions in Mammalian Cells.” Trends Biochem Sci 45(10): 858-873.
Chatenoud, L., C. Ferran, C. Legendre, I. Thouard, S. Merite, A. Reuter, Y. Gevaert, H. Kreis, P. Franchimont and J. F. Bach (1990). “In vivo cell activation following OKT3 administration. Systemic cytokine release and modulation by corticosteroids.” Transplantation 49(4): 697-702. Chen, X., J. L. Zaro and W. C. Shen (2013). “Fusion protein linkers: property, design and functionality.” Adv Drug Deliv Rev 65(10): 1357-1369.
Chini, E. N., C. C. S. Chini, J. M. Espindola Netto, G. C. de Oliveira and W. van Schooten (2018). “The Pharmacology of CD38/NADase: An Emerging Target in Cancer and Diseases of Aging.” Trends Pharmacol Sci 39(4): 424-436.
Chmielewski, J. P., S. C. Bowlby, F. B. Wheeler, L. Shi, G. Sui, A. L. Davis, T. D. Howard, R. B. D'Agostino, Jr., L. D. Miller, S. J. Sirintrapun, S. D. Cramer and S. J. Kridel (2018). “CD38 Inhibits Prostate Cancer Metabolism and Proliferation by Reducing Cellular NAD(+) Pools.” Mol Cancer Res 16(11): 1687-1700.
Chmielewski, M., A. Hombach, C. Heuser, G. P. Adams and H. Abken (2004). “T cell activation by antibody-like immunoreceptors: increase in affinity of the single-chain fragment domain above threshold does not increase T cell activation against antigen-positive target cells but decreases selectivity.” J Immunol 173(12): 7647-7653.
Chothia, C. and A. M. Lesk (1987). “Canonical structures for the hypervariable regions of immunoglobulins.” J Mol Biol 196(4): 901-917.
Chu, S. Y., I. Vostiar, S. Karki, G. L. Moore, G. A. Lazar, E. Pong, P. F. Joyce, D. E. Szymkowski and J. R. Desjarlais (2008). “Inhibition of B cell receptor-mediated activation of primary human B cells by coengagement of CD19 and FcgammaRIIb with Fc-engineered antibodies.” Mol Immunol 45(15): 3926-3933.
Covarrubias, A. J., A. Kale, R. Perrone, J. A. Lopez-Dominguez, A. O. Pisco, H. G. Kasler, M. S. Schmidt, I. Heckenbach, R. Kwok, C. D. Wiley, H. S. Wong, E. Gibbs, S. S. Iyer, N. Basisty, Q. Wu, I. J. Kim, E. Silva, K. Vitangcol, K. O. Shin, Y. M. Lee, R. Riley, I. Ben-Sahra, M. Ott, B. Schilling, M. Scheibye-Knudsen, K. Ishihara, S. R. Quake, J. Newman, C. Brenner, J. Campisi and E. Verdin (2020). “Senescent cells promote tissue NAD(+) decline during ageing via the activation of CD38(+) macrophages.” Nat Metab 2(11): 1265-1283.
Dall'Acqua, W. F., P. A. Kiener and H. Wu (2006). “Properties of human IgG1s engineered for enhanced binding to the neonatal Fc receptor (FcRn).” J Biol Chem 281(33): 23514-23524. Damle, R. N., T. Wasil, F. Fais, F. Ghiotto, A. Valetto, S. L. Allen, A. Buchbinder, D. Budman, K. Dittmar, J. Kolitz, S. M. Lichtman, P. Schulman, V. P. Vinciguerra, K. R. Rai, M. Ferrarini and N. Chiorazzi (1999). “Ig V gene mutation status and CD38 expression as novel prognostic indicators in chronic lymphocytic leukemia.” Blood 94(6): 1840-1847.
Deaglio, S., K. Mehta and F. Malavasi (2001). “Human CD38: a (r)evolutionary story of enzymes and receptors.” Leuk Res 25(1): 1-12.
Deshpande, D. A., A. G. P. Guedes, F. E. Lund, S. Subramanian, T. F. Walseth and M. S. Kannan (2017). “CD38 in the pathogenesis of allergic airway disease: Potential therapeutic targets.” Pharmacol Ther 172:116-126.
Diebolder, C. A., F. J. Beurskens, R. N. de Jong, R. I. Koning, K. Strumane, M. A. Lindorfer, M. Voorhorst, D. Ugurlar, S. Rosati, A. J. Heck, J. G. van de Winkel, I. A. Wilson, A. J. Koster, R. P. Taylor, E. O. Saphire, D. R. Burton, J. Schuurman, P. Gros and P. W. Parren (2014). “Complement is activated by IgG hexamers assembled at the cell surface.” Science 343(6176): 1260-1263.
Dimopoulos, M. A., A. Oriol, H. Nahi, J. San-Miguel, N. J. Bahlis, S. Z. Usmani, N. Rabin, R. Z. Orlowski, M. Komarnicki, K. Suzuki, T. Plesner, S. S. Yoon, D. Ben Yehuda, P. G. Richardson, H. Goldschmidt, D. Reece, S. Lisby, N. Z. Khokhar, L. O'Rourke, C. Chiu, X. Qin, M. Guckert, T. Ahmadi, P. Moreau and P. Investigators (2016). “Daratumumab, Lenalidomide, and Dexamethasone for Multiple Myeloma.” N Engl J Med 375(14): 1319-1331.
Drent, E., R. W. Groen, W. A. Noort, M. Themeli, J. J. Lammerts van Bueren, P. W. Parren, J. Kuball, Z. Sebestyen, H. Yuan, J. de Bruijn, N. W. van de Donk, A. C. Martens, H. M. Lokhorst and T. Mutis (2016). “Pre-clinical evaluation of CD38 chimeric antigen receptor engineered T cells for the treatment of multiple myeloma.” Haematologica 101(5): 616-625.
Drent, E., M. Themeli, R. Poels, R. de Jong-Korlaar, H. Yuan, J. de Bruijn, A. C. M. Martens, S. Zweegman, N. van de Donk, R. W. J. Groen, H. M. Lokhorst and T. Mutis (2017). “A Rational Strategy for Reducing On-Target Off-Tumor Effects of CD38-Chimeric Antigen Receptors by Affinity Optimization.” Mol Ther 25(8): 1946-1958.
Fayon, M., C. Martinez-Cingolani, A. Abecassis, N. Roders, E. Nelson, C. Choisy, A. Talbot, A. Bensussan, J. P. Fermand, B. Arnulf and J. C. Bories (2021). “Bi38-3 is a novel CD38/CD3 bispecific T-cell engager with low toxicity for the treatment of multiple myeloma.” Haematologica 106(4): 1193-1197.
Frankel, S. R. and P. A. Baeuerle (2013). “Targeting T cells to tumor cells using bispecific antibodies.” Curr Opin Chem Biol 17(3): 385-392.
Fumey, W., J. Koenigsdorf, V. Kunick, S. Menzel, K. Schutze, M. Unger, L. Schriewer, F. Haag, G. Adam, A. Oberle, M. Binder, R. Fliegert, A. Guse, Y. J. Zhao, H. Cheung Lee, F. Malavasi, F. Goldbaum, R. van Hegelsom, C. Stortelers, P. Bannas and F. Koch-Nolte (2017).
“Nanobodies effectively modulate the enzymatic activity of CD38 and allow specific imaging of CD38(+) tumors in mouse models in vivo.” Sci Rep 7(1): 14289.
Ge, Y., Y. Long, S. Xiao, L. Liang, Z. He, C. Yue, X. Wei and Y. Zhou (2019). “CD38 affects the biological behavior and energy metabolism of nasopharyngeal carcinoma cells.” Int J Oncol 54(2): 585-599.
Glaria, E. and A. F. Valledor (2020). “Roles of CD38 in the Immune Response to Infection.” Cells 9(1).
Guan, X. H., X. Hong, N. Zhao, X. H. Liu, Y. F. Xiao, T. T. Chen, L. B. Deng, X. L. Wang, J. B. Wang, G. J. Ji, M. Fu, K. Y. Deng and H. B. Xin (2017). “CD38 promotes angiotensin II-induced cardiac hypertrophy.” J Cell Mol Med 21(8): 1492-1502.
Guedes, A. G., M. Dileepan, J. A. Jude, D. A. Deshpande, T. F. Walseth and M. S. Kannan (2020). “Role of CD38/cADPR signaling in obstructive pulmonary diseases.” Curr Opin Pharmacol 51:29-33.
Guerreiro, S., A. L. Privat, L. Bressac and D. Toulorge (2020). “CD38 in Neurodegeneration and Neuroinflammation.” Cells 9(2).
Gunasekaran, K., M. Pentony, M. Shen, L. Garrett, C. Forte, A. Woodward, S. B. Ng, T. Born, M. Retter, K. Manchulenko, H. Sweet, I. N. Foltz, M. Wittekind and W. Yan (2010). “Enhancing antibody F_{c} heterodimer formation through electrostatic steering effects: applications to bispecific molecules and monovalent IgG.” J Biol Chem 285(25): 19637-19646.
Henriques, A., I. Silva, L. Ines, M. M. Souto-Carneiro, M. L. Pais, H. Trindade, J. A. da Silva and A. Paiva (2016). “CD38, CD81 and BAFFR combined expression by transitional B cells distinguishes active from inactive systemic lupus erythematosus.” Clin Exp Med 16(2): 227-232. Higashida, H. and T. Munesue (2013). “[CD38 and autism spectrum disorders].” No To Hattatsu 45(6): 431-435.
Hinton, P. R., J. M. Xiong, M. G. Johlfs, M. T. Tang, S. Keller and N. Tsurushita (2006). “An engineered human IgG1 antibody with longer serum half-life.” J Immunol 176(1): 346-356. Hipp, S., Y. T. Tai, D. Blanset, P. Deegen, J. Wahl, O. Thomas, B. Rattel, P. J. Adam, K. C. Anderson and M. Friedrich (2017). “A novel BCMA/CD3 bispecific T-cell engager for the treatment of multiple myeloma induces selective lysis in vitro and in vivo.” Leukemia 31(8): 1743-1751.
Hogan, K. A., C. C. S. Chini and E. N. Chini (2019). “The Multi-faceted Ecto-enzyme CD38: Roles in Immunomodulation, Cancer, Aging, and Metabolic Diseases.” Front Immunol 10:1187. Jiang, Z., D. Wu, S. Lin and P. Li (2016). “CD34 and CD38 are prognostic biomarkers for acute B lymphoblastic leukemia.” Biomark Res 4:23.
Jiao, Y., M. Yi, L. Xu, Q. Chu, Y. Yan, S. Luo and K. Wu (2020). “CD38: targeted therapy in multiple myeloma and therapeutic potential for solid cancers.” Expert Opin Investig Drugs 29(11): 1295-1308.
Joosse, M. E., C. L. Menckeberg, L. F. de Ruiter, H. R. C. Raatgeep, L. A. van Berkel, Y. Simons-Oosterhuis, I. Tindemans, A. F. M. Muskens, R. W. Hendriks, R. M. Hoogenboezem, T. Cupedo, L. de Ridder, J. C. Escher, S. Veenbergen and J. N. Samsom (2019). “Frequencies of circulating regulatory TIGIT(+)CD38(+) effector T cells correlate with the course of inflammatory bowel disease.” Mucosal Immunol 12(1): 154-163.
Kang, B. E., J. Y. Choi, S. Stein and D. Ryu (2020). “Implications of NAD(+) boosters in translational medicine.” Eur J Clin Invest 50(10): e13334.
Kar, A., S. Mehrotra and S. Chatterjee (2020). “CD38: T Cell Immuno-Metabolic Modulator.” Cells 9(7).
Karimi-Busheri, F., V. Zadorozhny, T. Li, H. Lin, D. L. Shawler and H. Fakhrai (2011). “Pivotal role of CD38 biomarker in combination with CD24, EpCAM, and ALDH for identification of H460 derived lung cancer stem cells.” J Stem Cells 6(1): 9-20.
Kim, B. J., Y. M. Choi, S. Y. Rah, D. R. Park, S. A. Park, Y. J. Chung, S. M. Park, J. K. Park, K. Y. Jang and U. H. Kim (2015). “Seminal CD38 is a pivotal regulator for fetomaternal tolerance.” Proc Natl Acad Sci USA 112(5): 1559-1564.
Kim, B. J., D. R. Park, T. S. Nam, S. H. Lee and U. H. Kim (2015). “Seminal CD38 Enhances Human Sperm Capacitation through Its Interaction with CD31.” PLOS One 10(9): e0139110. Kinder, M., A. R. Greenplate, K. D. Grugan, K. L. Soring, K. A. Heeringa, S. G. McCarthy, G. Bannish, M. Perpetua, F. Lynch, R. E. Jordan, W. R. Strohl and R. J. Brezski (2013). “Engineered protease-resistant antibodies with selectable cell-killing functions.” J Biol Chem 288(43): 30843-30854.
Klein, C., C. Sustmann, M. Thomas, K. Stubenrauch, R. Croasdale, J. Schanzer, U. Brinkmann, H. Kettenberger, J. T. Regula and W. Schaefer (2012). “Progress in overcoming the chain association issue in bispecific heterodimeric IgG antibodies.” MAbs 4(6): 653-663.
Labrijn, A. F., J. I. Meesters, B. E. de Goeij, E. T. van den Bremer, J. Neijssen, M. D. van Kampen, K. Strumane, S. Verploegen, A. Kundu, M. J. Gramer, P. H. van Berkel, J. G. van de Winkel, J. Schuurman and P. W. Parren (2014). “Efficient generation of stable bispecific IgG1 by controlled Fab-arm exchange.” Proc Natl Acad Sci USA 110(13): 5145-5150. Lam, J. H., H. H. M. Ng, C. J. Lim, X. N. Sim, F. Malavasi, H. Li, J. J. H. Loh, K. Sabai, J. K. Kim, C. C. H. Ong, T. Loh, W. Q. Leow, S. P. Choo, H. C. Toh, S. Y. Lee, C. Y. Chan, V. Chew, T. S. Lim, J. Yeong and T. K. H. Lim (2019). “Expression of CD38 on Macrophages Predicts Improved Prognosis in Hepatocellular Carcinoma.” Front Immunol 10:2093.
Lefranc, M. P., C. Pommie, M. Ruiz, V. Giudicelli, E. Foulquier, L. Truong, V. Thouvenin-Contet and G. Lefranc (2003). “IMGT unique numbering for immunoglobulin and T cell receptor variable domains and Ig superfamily V-like domains.” Dev Comp Immunol 27(1): 55-77. Li, J., N. J. Stagg, J. Johnston, M. J. Harris, S. A. Menzies, D. DiCara, V. Clark, M. Hristopoulos, R. Cook, D. Slaga, R. Nakamura, L. McCarty, S. Sukumaran, E. Luis, Z. Ye, T. D. Wu, T. Sumiyoshi, D. Danilenko, G. Y. Lee, K. Totpal, D. Ellerman, I. Hotzel, J. R. James and T. T. Junttila (2017). “Membrane-Proximal Epitope Facilitates Efficient T Cell Synapse Formation by Anti-FcRH5/CD3 and Is a Requirement for Myeloma Cell Killing.” Cancer Cell 31(3): 383-395.
Liao, S., S. Xiao, H. Chen, M. Zhang, Z. Chen, Y. Long, L. Gao, G. Zhu, J. He, S. Peng, W. Xiong, Z. Zeng, Z. Li, M. Zhou, X. Li, J. Ma, M. Wu, J. Xiang, G. Li and Y. Zhou (2017). “CD38 enhances the proliferation and inhibits the apoptosis of cervical cancer cells by affecting the mitochondria functions.” Mol Carcinog 56(10): 2245-2257.
Liao, S., S. Xiao, G. Zhu, D. Zheng, J. He, Z. Pei, G. Li and Y. Zhou (2014). “CD38 is highly expressed and affects the PI3K/Akt signaling pathway in cervical cancer.” Oncol Rep 32(6): 2703-2709.
Lin, W. W., Y. C. Lu, C. H. Chuang and T. L. Cheng (2020). “Ab locks for improving the selectivity and safety of antibody drugs.” J Biomed Sci 27(1): 76.
Lins, L., E. Farias, C. Brites-Alves, A. Torres, E. M. Netto and C. Brites (2017). “Increased expression of CD38 and HLADR in HIV-infected patients with oral lesion.” J Med Virol 89(10): 1782-1787.
Liu, X., T. R. Grogan, H. Hieronymus, T. Hashimoto, J. Mottahedeh, D. Cheng, L. Zhang, K. Huang, T. Stoyanova, J. W. Park, R. O. Shkhyan, B. Nowroozizadeh, M. B. Rettig, C. L. Sawyers, D. Elashoff, S. Horvath, J. Huang, O. N. Witte and A. S. Goldstein (2016). “Low CD38 Identifies Progenitor-like Inflammation-Associated Luminal Cells that Can Initiate Human Prostate Cancer and Predict Poor Outcome.” Cell Rep 17(10): 2596-2606.
Lopatina, O., A. Inzhutova, A. B. Salmina and H. Higashida (2012). “The roles of oxytocin and CD38 in social or parental behaviors.” Front Neurosci 6:182.
Ma, S. R., W. W. Deng, J. F. Liu, L. Mao, G. T. Yu, L. L. Bu, A. B. Kulkarni, W. F. Zhang and Z. J. Sun (2017). “Blockade of adenosine A2A receptor enhances CD8(+) T cells response and decreases regulatory T cells in head and neck squamous cell carcinoma.” Mol Cancer 16(1): 99. Malavasi, F., S. Deaglio, R. Damle, G. Cutrona, M. Ferrarini and N. Chiorazzi (2011). “CD38 and chronic lymphocytic leukemia: a decade later.” Blood 118(13): 3470-3478.
Malavasi, F., S. Deaglio, A. Funaro, E. Ferrero, A. L. Horenstein, E. Ortolan, T. Vaisitti and S. Aydin (2008). “Evolution and function of the ADP ribosyl cyclase/CD38 gene family in physiology and pathology.” Physiol Rev 88(3): 841-886.
Malavasi, F., A. Funaro, S. Roggero, A. Horenstein, L. Calosso and K. Mehta (1994). “Human CD38: a glycoprotein in search of a function.” Immunol Today 15(3): 95-97.
Mallone, R., E. Ortolan, S. Pinach, M. Volante, M. M. Zanone, G. Bruno, G. Baj, T. Lohmann, P. Cavallo-Perin and F. Malavasi (2002). “Anti-CD38 autoantibodies: characterisation in new-onset type I diabetes and latent autoimmune diabetes of the adult (LADA) and comparison with other islet autoantibodies.” Diabetologia 45(12): 1667-1677.
Mallone, R. and P. C. Perin (2006). “Anti-CD38 autoantibodies in type? diabetes.” Diabetes Metab Res Rev 22(4): 284-294.
Marlein, C. R., R. E. Piddock, J. J. Mistry, L. Zaitseva, C. Hellmich, R. H. Horton, Z. Zhou, M. J. Auger, K. M. Bowles and S. A. Rushworth (2019). “CD38-Driven Mitochondrial Trafficking Promotes Bioenergetic Plasticity in Multiple Myeloma.” Cancer Res 79(9): 2285-2297.
May, C., P. Sapra and H. P. Gerber (2012). “Advances in bispecific biotherapeutics for the treatment of cancer.” Biochem Pharmacol 84(9): 1105-1112.
Mehta, K., U. Shahid and F. Malavasi (1996). “Human CD38, a cell-surface protein with multiple functions.” FASEB J 10(12): 1408-1417.
Moore, G. L., C. Bautista, E. Pong, D. H. Nguyen, J. Jacinto, A. Eivazi, U. S. Muchhal, S. Karki, S. Y. Chu and G. A. Lazar (2011). “A novel bispecific antibody format enables simultaneous bivalent and monovalent co-engagement of distinct target antigens.” MAbs 3(6): 546-557.
Morandi, F., I. Airoldi, D. Marimpietri, C. Bracci, A. C. Faini and R. Gramignoli (2019). “CD38, a Receptor with Multifunctional Activities: From Modulatory Functions on Regulatory Cell Subsets and Extracellular Vesicles, to a Target for Therapeutic Strategies.” Cells 8(12).
Munoz, P., M. Mittelbrunn, H. de la Fuente, M. Perez-Martinez, A. Garcia-Perez, A. Ariza-Veguillas, F. Malavasi, M. Zubiaur, F. Sanchez-Madrid and J. Sancho (2008). “Antigen-induced clustering of surface CD38 and recruitment of intracellular CD38 to the immunologic synapse.” Blood 111(7): 3653-3664.
Nooka, A. K., J. L. Kaufman, C. C. Hofmeister, N. S. Joseph, T. L. Heffner, V. A. Gupta, H. C. Sullivan, A. S. Neish, M. V. Dhodapkar and S. Lonial (2019). “Daratumumab in multiple myeloma.” Cancer 125(14): 2364-2382.
Orciani, M., O. Trubiani, S. Guarnieri, E. Ferrero and R. Di Primio (2008). “CD38 is constitutively expressed in the nucleus of human hematopoietic cells.” J Cell Biochem 105(3): 905-912.
Palumbo, A., A. Chanan-Khan, K. Weisel, A. K. Nooka, T. Masszi, M. Beksac, I. Spicka, V. Hungria, M. Munder, M. V. Mateos, T. M. Mark, M. Qi, J. Schecter, H. Amin, X. Qin, W. Deraedt, T. Ahmadi, A. Spencer, P. Sonneveld and C. Investigators (2016). “Daratumumab, Bortezomib, and Dexamethasone for Multiple Myeloma.” N Engl J Med 375(8): 754-766.
Perenkov, A. D., D. V. Novikov, N. A. Sakharnov, A. V. Aliasova, O. V. Utkin, A. Baryshnikov and V. V. Novikov (2012). “[Heterogeneous expression of CD38 gene in tumor tissue in patients with colorectal cancer].” Mol Biol (Mosk) 46(5): 786-791.
Petkova, S. B., S. Akilesh, T. J. Sproule, G. J. Christianson, H. Al Khabbaz, A. C. Brown, L. G. Presta, Y. G. Meng and D. C. Roopenian (2006). “Enhanced half-life of genetically engineered human IgG1 antibodies in a humanized FcRn mouse model: potential application in humorally mediated autoimmune disease.” Int Immunol 18(12): 1759-1769.
Quarona, V., G. Zaccarello, A. Chillemi, E. Brunetti, V. K. Singh, E. Ferrero, A. Funaro, A. L. Horenstein and F. Malavasi (2013). “CD38 and CD157: a long journey from activation markers to multifunctional molecules.” Cytometry B Clin Cytom 84(4): 207-217.
Ridgway, J. B., L. G. Presta and P. Carter (1996). “Knobs-into-holes” engineering of antibody CH3 domains for heavy chain heterodimerization.” Protein Eng 9(7): 617-621. Salmina, A. B., O. Lopatina, N. V. Kuvacheva and H. Higashida (2013). “Integrative neurochemistry and neurobiology of social recognition and behavior analyzed with respect to CD38-dependent brain oxytocin secretion.” Curr Top Med Chem 13(23): 2965-2977.
Saltarella, I., V. Desantis, A. Melaccio, A. G. Solimando, A. Lamanuzzi, R. Ria, C. T. Storlazzi, M. A. Mariggio, A. Vacca and M. A. Frassanito (2020). “Mechanisms of Resistance to Anti-CD38 Daratumumab in Multiple Myeloma.” Cells 9(1).
Sanchez, E., M. Li, A. Kitto, J. Li, C. S. Wang, D. T. Kirk, O. Yellin, C. M. Nichols, M. P. Dreyer, C. P. Ahles, A. Robinson, E. Madden, G. N. Waterman, R. A. Swift, B. Bonavida, R. Boccia, R. A. Vescio, J. Crowley, H. Chen and J. R. Berenson (2012). “Serum B-cell maturation antigen is elevated in multiple myeloma and correlates with disease status and survival.” Br J Haematol 158(6): 727-738.
Scheraga, H. A. (2008). “From helix-coil transitions to protein folding.” Biopolymers 89(5): 479-485.
Schiavoni, I., C. Scagnolari, A. L. Horenstein, P. Leone, A. Pierangeli, F. Malavasi, C. M. Ausiello and G. Fedele (2018). “CD38 modulates respiratory syncytial virus-driven proinflammatory processes in human monocyte-derived dendritic cells.” Immunology 154(1): 122-131.
Schultz, M. B. and D. A. Sinclair (2016). “Why NAD(+) Declines during Aging: It's Destroyed.” Cell Metab 23(6): 965-966.
Schutze, K., K. Petry, J. Hambach, N. Schuster, W. Fumey, L. Schriewer, J. Rockendorf, S. Menzel, B. Albrecht, F. Haag, C. Stortelers, P. Bannas and F. Koch-Nolte (2018). “CD38-Specific Biparatopic Heavy Chain Antibodies Display Potent Complement-Dependent Cytotoxicity Against Multiple Myeloma Cells.” Front Immunol 9:2553.
Seckinger, A., J. A. Delgado, S. Moser, L. Moreno, B. Neuber, A. Grab, S. Lipp, J. Merino, F. Prosper, M. Emde, C. Delon, M. Latzko, R. Gianotti, R. Luoend, R. Murr, R. J. Hosse, L. J. Harnisch, M. Bacac, T. Fauti, C. Klein, A. Zabaleta, J. Hillengass, E. A. Cavalcanti-Adam, A. D. Ho, M. Hundemer, J. F. San Miguel, K. Strein, P. Umana, D. Hose, B. Paiva and M. D. Vu (2017). “Target Expression, Generation, Preclinical Activity, and Pharmacokinetics of the BCMA-T Cell Bispecific Antibody EM801 for Multiple Myeloma Treatment.” Cancer Cell 31(3): 396-410.
Shields, R. L., A. K. Namenuk, K. Hong, Y. G. Meng, J. Rae, J. Briggs, D. Xie, J. Lai, A. Stadlen, B. Li, J. A. Fox and L. G. Presta (2001). “High resolution mapping of the binding site on human IgG1 for Fc gamma RI, Fc gamma RII, Fc gamma RIII, and FcRn and design of IgG1 variants with improved binding to the Fc gamma R.” J Biol Chem 276(9): 6591-6604.
Shields, R. L., A. K. Namenuk, K. Hong, Y. G. Meng, J. Rae, J. Briggs, D. Xie, J. Lai, A. Stadlen, B. Li, J. A. Fox and L. G. Presta (2001). “High resolution mapping of the binding site on human IgG1 for Fc gamma RI, Fc gamma RII, Fc gamma RIII, and FcRn and design of IgG1 variants with improved binding to the Fc gamma R.” J Biol Chem 276(9): 6591-6604.
Stone, L. (2017). “Prostate cancer: On the down-low—low luminal cell CD38 expression is prognostic.” Nat Rev Urol 14(3): 133.
Su, A. I., T. Wiltshire, S. Batalov, H. Lapp, K. A. Ching, D. Block, J. Zhang, R. Soden, M. Hayakawa, G. Kreiman, M. P. Cooke, J. R. Walker and J. B. Hogenesch (2004). “A gene atlas of the mouse and human protein-encoding transcriptomes.” Proc Natl Acad Sci USA 101(16): 6062-6067.
Tam, S. H., S. G. McCarthy, A. A. Armstrong, S. Somani, S. J. Wu, X. Liu, A. Gervais, R. Ernst, D. Saro, R. Decker, J. Luo, G. L. Gilliland, M. L. Chiu and B. J. Scallon (2017). “Functional, Biophysical, and Structural Characterization of Human IgG1 and IgG4 Fc Variants with Ablated Immune Functionality.” Antibodies (Basel) 6(3).
Usmani, S. Z., B. M. Weiss, T. Plesner, N. J. Bahlis, A. Belch, S. Lonial, H. M. Lokhorst, P. M. Voorhees, P. G. Richardson, A. Chari, A. K. Sasser, A. Axel, H. Feng, C. M. Uhlar, J. Wang, I. Khan, T. Ahmadi and H. Nahi (2016). “Clinical efficacy of daratumumab monotherapy in patients with heavily pretreated relapsed or refractory multiple myeloma.” Blood 128(1): 37-44.
Vafa, O., G. L. Gilliland, R. J. Brezski, B. Strake, T. Wilkinson, E. R. Lacy, B. Scallon, A. Teplyakov, T. J. Malia and W. R. Strohl (2014). “An engineered F_{c} variant of an IgG eliminates all immune effector functions via structural perturbations.” Methods 65(1): 114-126. van de Donk, N. and S. Z. Usmani (2018). “CD38 Antibodies in Multiple Myeloma: Mechanisms of Action and Modes of Resistance.” Front Immunol 9:2134.
Wang, H., G. Kaur, A. I. Sankin, F. Chen, F. Guan and X. Zang (2019). “Immune checkpoint blockade and CAR-T cell therapy in hematologic malignancies.” J Hematol Oncol 12(1): 59. Wang, H., S. Li, G. Zhang, H. Wu and X. Chang (2019). “Potential therapeutic effects of cyanidin-3-O-glucoside on rheumatoid arthritis by relieving inhibition of CD38+NK cells on Treg cell differentiation.” Arthritis Res Ther 21(1): 220.
Wang, L., S. S. Hoseini, H. Xu, V. Ponomarev and N. K. Cheung (2019). “Silencing F_{c} Domains in T cell-Engaging Bispecific Antibodies Improves T-cell Trafficking and Antitumor Potency.” Cancer Immunol Res 7(12): 2013-2024. Wikipedia. (2021). “Catumaxomab.”
Worn, A. and A. Pluckthun (2001). “Stability engineering of antibody single-chain Fv fragments.” J Mol Biol 305(5): 989-1010.
Wu, C., X. Jin, G. Tsueng, C. Afrasiabi and A. I. Su (2016). “BioGPS: building your own mash-up of gene annotations and expression profiles.” Nucleic Acids Res 44 (D1): D313-316. Wu, C., L. Zhang, Q. R. Brockman, F. Zhan and L. Chen (2019). “Chimeric antigen receptor T cell therapies for multiple myeloma.” J Hematol Oncol 12(1): 120.
Wu, T. T. and E. A. Kabat (1970). “An analysis of the sequences of the variable regions of Bence Jones proteins and myeloma light chains and their implications for antibody complementarity.” J Exp Med 132(2): 211-250.
Wursch, D., C. E. Ormsby, D. P. Romero-Rodriguez, G. Olvera-Garcia, J. Zuniga, W. Jiang, S. Perez-Patrigeon and E. Espinosa (2016). “CD38 Expression in a Subset of Memory T Cells Is Independent of Cell Cycling as a Correlate of HIV Disease Progression.” Dis Markers 2016:9510756.
Xu, D., M. L. Alegre, S. S. Varga, A. L. Rothermel, A. M. Collins, V. L. Pulito, L. S. Hanna, K. P. Dolan, P. W. Parren, J. A. Bluestone, L. K. Jolliffe and R. A. Zivin (2000). “In vitro characterization of five humanized OKT3 effector function variant antibodies.” Cell Immunol 200(1): 16-26.
Xu, L., D. Chen, C. Lu, X. Liu, G. Wu and Y. Zhang (2015). “Advanced Lung Cancer Is Associated with Decreased Expression of Perforin, CD95, CD38 by Circulating CD3+CD8+ T Lymphocytes.” Ann Clin Lab Sci 45(5): 528-532.
Yarbro, J. R., R. S. Emmons and B. D. Pence (2020). “Macrophage Immunometabolism and Inflammaging: Roles of Mitochondrial Dysfunction, Cellular Senescence, CD38, and NAD.” Immunometabolism 2(3): e200026.
Yeong, J., J. C. T. Lim, B. Lee, H. Li, N. Chia, C. C. H. Ong, W. K. Lye, T. C. Putti, R. Dent, E. Lim, A. A. Thike, P. H. Tan and J. Iqbal (2018). “High Densities of Tumor-Associated Plasma Cells Predict Improved Prognosis in Triple Negative Breast Cancer.” Front Immunol 9:1209. Yu, S., M. Yi, S. Qin and K. Wu (2019). “Next generation chimeric antigen receptor T cells: safety strategies to overcome toxicity.” Mol Cancer 18(1): 125.
Zalevsky, J., A. K. Chamberlain, H. M. Horton, S. Karki, I. W. Leung, T. J. Sproule, G. A. Lazar, D. C. Roopenian and J. R. Desjarlais (2010). “Enhanced antibody half-life improves in vivo activity.” Nat Biotechnol 28(2): 157-159.
Zambello, R., G. Barila, S. Manni, F. Piazza and G. Semenzato (2020). “NK cells and CD38: Implication for (Immuno) Therapy in Plasma Cell Dyscrasias.” Cells 9(3).
Zeijlemaker, W., T. Grob, R. Meijer, D. Hanekamp, A. Kelder, J. C. Carbaat-Ham, Y. J. M. Oussoren-Brockhoff, A. N. Snel, D. Veldhuizen, W. J. Scholten, J. Maertens, D. A. Breems, T. Pabst, M. G. Manz, V. H. J. van der Velden, J. Slomp, F. Preijers, J. Cloos, A. A. van de Loosdrecht, B. Lowenberg, P. J. M. Valk, M. Jongen-Lavrencic, G. J. Ossenkoppele and G. J. Schuurhuis (2019). “CD34(+) CD38(−) leukemic stem cell frequency to predict outcome in acute myeloid leukemia.” Leukemia 33(5): 1102-1112.
Zhang, D., A. A. Armstrong, S. H. Tam, S. G. McCarthy, J. Luo, G. L. Gilliland and M. L. Chiu (2017). “Functional optimization of agonistic antibodies to OX40 receptor with novel Fc mutations to promote antibody multimerization.” MAbs 9:1129-1142.
Zhang, M., J. Yang, J. Zhou, W. Gao, Y. Zhang, Y. Lin, H. Wang, Z. Ruan and B. Ni (2019). “Prognostic Values of CD38(+) CD101(+) PD1(+) CD8(+) T Cells in Pancreatic Cancer.” Immunol Invest 48(5): 466-479.
Zheng, D., S. Liao, G. Zhu, G. Luo, S. Xiao, J. He, Z. Pei, G. Li and Y. Zhou (2016). “CD38 is a putative functional marker for side population cells in human nasopharyngeal carcinoma cell lines.” Mol Carcinog 55(3): 300-311.
Zuch de Zafra, C. L., F. Fajardo, W. Zhong, M. J. Bernett, U. S. Muchhal, G. L. Moore, J. Stevens, R. Case, J. T. Pearson, S. Liu, P. L. McElroy, J. Canon, J. R. Desjarlais, A. Coxon, M. Balazs and O. Nolan-Stevaux (2019). “Targeting Multiple Myeloma with AMG 424, a Novel Anti-CD38/CD3 Bispecific T-cell-recruiting Antibody Optimized for Cytotoxicity and Cytokine Release.” Clin Cancer Res 25(13): 3921-3933.

1. An anti-CD38 antibody or an antigen-binding fragment thereof, comprising:
a heavy chain variable region comprising HCDR1, HCDR2, and HCDR3, wherein the HCDR1, HCDR2, and HCDR3 are selected from: SEQ ID NOs: 64, 65, and 66; SEQ ID NOs: 64, 83, and 84; SEQ ID NOs: 64, 65, and 85; and SEQ ID NOs: 64, 65, and 86; respectively; and
a light chain variable region comprising LCDR1, LCDR2, and LCDR3, wherein the LCDR1, LCDR2, and LCDR3 are selected from: SEQ ID NOs: 61, 62, and 63; SEQ ID NOs: 76, 77, and 78; SEQ ID NOs: 79, 80, and 81; and SEQ ID NOs: 82, 62, and 78; respectively.

2. The anti-CD38 antibody or antigen binding fragment of claim 1, comprising a heavy chain sequence comprising an amino acid sequence having at least 85% identity to any one of SEQ ID NOs: 12-16 or an antigen-binding fragment thereof, and a light chain sequence comprising an amino acid sequence having at least 85% identity to any one of SEQ ID NOs: 7-11 or an antigen-binding fragment thereof.

3. An anti-CD38 antibody or an antigen binding fragment thereof, comprising a light chain sequence and a heavy chain sequence comprising: SEQ ID NOs: 2 and 3; SEQ ID NOs: 7 and 12; SEQ ID NOs: 7 and 13; SEQ ID NOs: 7 and 14; SEQ ID NOs: 7 and 15; SEQ ID NOs: 7 and 16; SEQ ID NOs: 8 and 12; SEQ ID NOs: 8 and 13; SEQ ID NOs: 8 and 14; SEQ ID NOs: 8 and 15; SEQ ID NOs: 8 and 16; SEQ ID NOs: 9 and 12; SEQ ID NOs: 9 and 13; SEQ ID NOs: 9 and 14; SEQ ID NOs: 9 and 15; SEQ ID NOs: 9 and 16; SEQ ID NOs: 10 and 12; SEQ ID NOs: 10 and 13; SEQ ID NOs: 10 and 14; SEQ ID NOs: 10 and 15; SEQ ID NOs: 10 and 16; SEQ ID NOs: 11 and 12; SEQ ID NOs: 11 and 13; SEQ ID NOs: 11 and 14; SEQ ID NOs: 11 and 15; or SEQ ID NOs: 11 and 16; respectively.

4. An anti-CD38 antibody or an antigen-binding fragment thereof, comprising: at least one variable-heavy-chain-only single-domain or an antigen-binding fragment thereof, wherein the at least one variable-heavy-chain-only single-domain comprises HCDR1, HCDR2, and HCDR3 selected from: SEQ ID NOs: 67, 68, and 69; SEQ ID NOs: 67, 87, and 69; SEQ ID NOs: 67, 88, and 69; SEQ ID NOs: 67, 89, and 69; SEQ ID NOs: 67, 68, and 90; SEQ ID NOs: 70, 91, and 72; SEQ ID NOs: 70, 92, and 72; SEQ ID NOs: 70, 93, and 72; SEQ ID NOs: 94, 95, and 96; SEQ ID NOs: 70, 71, and 97; SEQ ID NOs: 73, 98, and 75; SEQ ID NOs: 73, 99, and 75; SEQ ID NOs: 73, 100, and 75; SEQ ID NOs: 73, 101, and 102; and SEQ ID NOs: 73, 103, and 104; respectively.

5. The anti-CD38 antibody or antigen binding fragment of claim 4, comprising at least one variable-heavy-chain-only single-domain or an antigen-binding fragment thereof, wherein the at least one variable-heavy-chain-only single-domain comprises an amino acid sequence having at least 85% identity to any one of SEQ ID NOs: 4 and 17-30 or an antigen-binding fragment thereof.

6. An anti-CD3 antibody or an antigen-binding fragment thereof, comprising: a heavy chain variable region comprising HCDR1, HCDR2, and HCDR3, wherein the HCDR1, HCDR2, and HCDR3 are selected from: SEQ ID NOs: 105, 108, and 107; SEQ ID NOs: 105, 109, and 107; SEQ ID NOs: 105, 110, and 107; and SEQ ID NOs: 118, 119, and 120; respectively; and a light chain variable region comprising LCDR1, LCDR2, and LCDR3, wherein the LCDR1, LCDR2, and LCDR3 are selected from: SEQ ID NOs: 114, 112, and 113; and SEQ ID NOs: 115, 116, and 117; respectively.

7. The anti-CD3 antibody or antigen binding fragment of claim 6, comprising a heavy chain sequence comprising an amino acid sequence having at least 85% identity to any one of SEQ ID NOs: 32-34 and 39 or an antigen-binding fragment thereof, and a light chain sequence comprising an amino acid sequence having at least 85% identity to any one of SEQ ID NOs: 36-38 or an antigen-binding fragment thereof.

8. An anti-CD3 antibody or an antigen binding fragment thereof, comprising a light chain sequence and a heavy chain sequence comprising: SEQ ID NOs: 39 and 38; SEQ ID NOs: 41 and 40; SEQ ID NOs: 31 and 35; SEQ ID NOs: 31 and 36; SEQ ID NOs: 31 and 37; SEQ ID NOs: 32 and 35; SEQ ID NOs: 32 and 36; SEQ ID NOs: 32 and 37; SEQ ID NOs: 33 and 35; SEQ ID NOs: 33 and 36; SEQ ID NOs: 33 and 37; SEQ ID NOs: 34 and 35; SEQ ID NOs: 34 and 36; or SEQ ID NOs: 34 and 37; respectively.

9. An anti-CD3 and anti-CD38 bispecific antibody, comprising:
(1) a first binding arm comprising:
a first heavy chain fusion protein comprising, from the N- to the C-terminus, an optional shield A, an optional protease sequence A, and an IgG heavy chain or an antigen-binding fragment thereof, and
a first light chain fusion protein comprising, from the N- to the C-terminus, an optional shield B, an optional protease sequence B, and an IgG light chain or an antigen-binding fragment thereof,
wherein:
the IgG heavy chain or antigen-binding fragment thereof of the first binding arm comprises HCDR1, HCDR2, and HCDR3 selected from: SEQ ID NOs: 105, 108, and 107; SEQ ID NOs: 105, 109, and 107; SEQ ID NOs: 105, 110, and 107; and SEQ ID NOs: 118, 119, and 120; respectively, and
the IgG light chain or antigen-binding fragment thereof of the first binding arm comprises LCDR1, LCDR2, and LCDR3 selected from: SEQ ID NOs: 114, 112, and 113; and SEQ ID NOs: 115, 116, and 117; respectively; and
(2) a second binding arm comprising:
a second heavy chain fusion protein comprising, from the N- to the C-terminus, an optional shield C, an optional protease sequence C, and an IgG heavy chain or an antigen-binding fragment thereof; and
a second light chain fusion protein comprising, from the N- to the C-terminus, an optional shield D, an optional protease sequence D, and an IgG light chain or an antigen-binding fragment thereof,
wherein:
the IgG heavy chain or antigen-binding fragment thereof of the second binding arm comprises HCDR1, HCDR2, and HCDR3, selected from: SEQ ID NOs: 64, 65, and 66; SEQ ID NOs: 64, 83, and 84; SEQ ID NOs: 64, 65, and 85; and SEQ ID NOs: 64, 65, and 86; respectively; and
the IgG light chain or antigen-binding fragment thereof of the second binding arm comprises LCDR1, LCDR2, and LCDR3, selected from: SEQ ID NOs: 61, 62, and 63; SEQ ID NOs: 76, 77, and 78; SEQ ID NOs: 79, 80, and 81; and SEQ ID NOs: 82, 62, and 78; respectively;
and
wherein the shields A-D are the same or different from one another, and protease sequences A-D are the same or different from one another.

10. The anti-CD3 and anti-CD38 bispecific antibody bispecific antibody of claim 9, wherein:
the IgG heavy chain or antigen-binding fragment thereof of the first binding arm comprises an amino acid sequence having at least 85% identity to any one of SEQ ID NOs: 32-34 and 39 or an antigen-binding fragment thereof,
the IgG light chain or antigen-binding fragment thereof of the first binding arm comprises a light chain sequence comprising an amino acid sequence having at least 85% identity to any one of SEQ ID NOs: 36-38 or an antigen-binding fragment thereof; and
wherein:
the IgG heavy chain or antigen-binding fragment thereof of the second binding arm comprises a heavy chain sequence comprising an amino acid sequence having at least 85% identity to any one of SEQ ID NOs: 12-16 or an antigen-binding fragment thereof, and
the IgG light chain or antigen-binding fragment thereof of the second binding arm comprises a light chain sequence comprising an amino acid sequence having at least 85% identity to any one of SEQ ID NOs: 7-11 or an antigen-binding fragment thereof.

11. A anti-CD3 and anti-CD38 bispecific antibody comprising:
(1) a first binding arm comprising:
a first heavy chain fusion protein comprising, from the N- to the C-terminus, an optional shield A, an optional protease sequence A, and an IgG heavy chain or an antigen-binding fragment thereof, and
a first light chain fusion protein comprising, from the N- to the C-terminus, an optional shield B, an optional protease sequence B, and an IgG light chain or an antigen-binding fragment thereof,
wherein:
the IgG heavy chain or antigen-binding fragment thereof of the first binding arm comprises HCDR1, HCDR2, and HCDR3 selected from: SEQ ID NOs: 105, 108, and 107; SEQ ID NOs: 105, 109, and 107; SEQ ID NOs: 105, 110, and 107; and SEQ ID NOs: 118, 119, and 120; respectively, and
the IgG light chain or antigen-binding fragment thereof of the first binding arm comprises LCDR1, LCDR2, and LCDR3 selected from: SEQ ID NOs: 114, 112, and 113; and SEQ ID NOs: 115, 116, and 117; respectively; and
(2) a second binding arm comprising:
a second heavy chain fusion protein comprising, from the N- to the C-terminus, an optional shield C, an optional protease sequence C, and an IgG heavy chain comprising at least one variable-heavy-chain-only single domain or an antigen-binding fragment thereof,
wherein the at least one variable-heavy-chain-only single domain or antigen-binding fragment thereof comprises HCDR1, HCDR2, and HCDR3 selected from: SEQ ID NOs: 67, 68, and 69; SEQ ID NOs: 67, 87, and 69; SEQ ID NOs: 67, 88, and 69; SEQ ID NOs: 67, 89, and 69; SEQ ID NOs: 67, 68, and 90; SEQ ID NOs: 70, 91, and 72; SEQ ID NOs: 70, 92, and 72; SEQ ID NOs: 70, 93, and 72; SEQ ID NOs: 94, 95, and 96; SEQ ID NOs: 70, 71, and 97; SEQ ID NOs: 73, 98, and 75; SEQ ID NOs: 73, 99, and 75; SEQ ID NOs: 73, 100, and 75; SEQ ID NOs: 73, 101, and 102; and SEQ ID NOs: 73, 103, and 104; respectively, and
wherein the shields A-C are the same or different from one another, and protease sequences A-C are the same or different from one another.

12. The anti-CD3 and anti-CD38 bispecific antibody of claim 11, wherein:
the IgG heavy chain or antigen-binding fragment thereof of the first binding arm comprises an amino acid sequence having at least 85% identity to any one of SEQ ID NOs: 32-34 and 39 or an antigen-binding fragment thereof,
the IgG light chain or antigen-binding fragment thereof of the first binding arm comprises a light chain sequence comprising an amino acid sequence having at least 85% identity to any one of SEQ ID NOs: 36-38 or an antigen-binding fragment thereof; and
wherein:
the at least one variable-heavy-chain-only single domain or an antigen-binding fragment thereof comprises an amino acid sequence with at least 85% identity to any one of SEQ ID NOs:
4 and 17-30 or antigen-binding fragments thereof.

13. The bispecific antibody of claim 11, wherein the first binding arm is monovalent, and the second binding arm is monovalent, bivalent, or multivalent.

14. The bispecific antibody of claim 11, wherein the second binding arm comprises two or three IgG variable-heavy-chain-only single domains in tandem, wherein the two or three IgG variable-heavy-chain-only single domains are optionally connected via one or more linker sequences.

15. The bispecific antibody of claim 9, wherein the shield A, shield B, shield C, and shield D are each independently selected from the amino acid sequences set forth in SEQ ID NOs: 42-52.

16. The bispecific antibody of claim 11, wherein the protease sequence A, protease sequence B, protease sequence C, and protease sequence D are each independently selected from amino acid sequences set forth in SEQ ID NOs: 53-60.

17. A bispecific antibody comprising a CD3-targeting binding arm, wherein the CD3-targeting binding arm comprises a heavy chain sequence and a light chain sequence comprising: SEQ ID NOs: 39 and 38; SEQ ID NOs: 41 and 40; SEQ ID NOs: 31 and 35; SEQ ID NOs: 31 and 36; SEQ ID NOs: 31 and 37; SEQ ID NOs: 32 and 35; SEQ ID NOs: 32 and 36; SEQ ID NOs: 32 and 37; SEQ ID NOs: 33 and 35; SEQ ID NOs: 33 and 36; SEQ ID NOs: 33 and 37; SEQ ID NOs: 34 and 35; SEQ ID NOs: 34 and 36; or SEQ ID NOs: 34 and 37; respectively.

18. The bispecific antibody of claim 17, further comprising a CD38-targeting binding arm, wherein the CD38-targeting binding arm comprises a heavy chain sequence and a light chain sequence comprising: SEQ ID NOs: 2 and 3; SEQ ID NOs: 7 and 12; SEQ ID NOs: 7 and 13; SEQ ID NOs: 7 and 14; SEQ ID NOs: 7 and 15; SEQ ID NOs: 7 and 16; SEQ ID NOs: 8 and 12; SEQ ID NOs: 8 and 13; SEQ ID NOs: 8 and 14; SEQ ID NOs: 8 and 15; SEQ ID NOs: 8 and 16; SEQ ID NOs: 9 and 12; SEQ ID NOs: 9 and 13; SEQ ID NOs: 9 and 14; SEQ ID NOs: 9 and 15; SEQ ID NOs: 9 and 16; SEQ ID NOs: 10 and 12; SEQ ID NOs: 10 and 13; SEQ ID NOs: 10 and 14; SEQ ID NOs: 10 and 15; SEQ ID NOs: 10 and 16; SEQ ID NOs: 11 and 12; SEQ ID NOs: 11 and 13; SEQ ID NOs: 11 and 14; SEQ ID NOs: 11 and 15; or SEQ ID NOs: 11 and 16, respectively.

19. The bispecific antibody of claim 17, further comprising a CD38-targeting binding arm, wherein the CD38-targeting binding arm comprises two variable-heavy-chain-only single domain comprising from the N- to the C-terminus, SEQ ID NO: 4 and SEQ ID NO: 5 optionally connected via a linker, SEQ ID NO: 4 and SEQ ID NO: 6 optionally connected via a linker, SEQ ID NO: 5 and SEQ ID NO: 6 optionally connected via a linker, SEQ ID NO: 5 and SEQ ID NO: 4 optionally connected via a linker, SEQ ID NO: 6 and SEQ ID NO: 5 optionally connected via a linker, or SEQ ID NO: 6 and SEQ ID NO: 4 optionally connected via a linker.

20. A conjugate comprising the anti-CD38 antibody or antigen binding fragment of claim 1 conjugated to a cytotoxic agent.

21. A pharmaceutical composition comprising the anti-CD38 antibody or antigen binding fragment of claim 1, and a pharmaceutically acceptable carrier.

22. A nucleic acid encoding the anti-CD38 antibody or antigen binding fragment of claim 1.

23. A host cell comprising the nucleic acid of claim 22.

24. A method for preparing an anti-CD38 antibody or antigen binding fragment, comprising: culturing a host cell comprising a nucleic acid encoding the anti-CD38 antibody or antigen binding fragment of claim 1, growing the host cell in a host cell culture, providing host cell culture conditions wherein the nucleic acid is expressed, and recovering the antibody or bispecific antibody from the host cell or from the host cell culture.

25. A method for treating or preventing a CD38-mediated disease or disorder in a subject in need thereof, comprising administering to the subject a pharmaceutically effective amount of the anti-CD38 antibody or antigen binding fragment of claim 1.

26. The method for treating or preventing CD38-mediated disease or disorder of claim 25, wherein the CD38-mediated disease or disorder is selected from gastric cancer, and colorectal cancer, pancreatic cancer, prostate cancer, lung cancer, hepatocellular cancer, triple-negative breast cancer, nasopharyngeal cancer, cervical cancer, and hematologic malignancies.


SEQUENCE LISTING (141 sequences)

SEQ ID NO: 1 - protein, 300 aa, Homo sapiens
  source 1..300: note = Human CD38
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 107 aa, synthetic construct [sequence omitted]

SEQ ID NO: 3 - protein, 120 aa, synthetic construct [sequence omitted]

SEQ ID NO: 4 - protein, 128 aa, synthetic construct
  REGION 1..128: note = CD38 5FIO VHO1
EVQLQESGGGLVQAGHSLRLSCVGSGSRFDNYAMGWFRQAPGKEREFVAAISWSSGTTRYLDTVKGRFTISRDNAKSTVYLQMNSLKPEDTAVYYCAARYQPRYYDSGDMDGYEYDNWGQGTQVTVSS

SEQ ID NO: 5 - protein, 124 aa, synthetic construct
  REGION 1..124: note = CD38 5F1K VHO1
EVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGIVAGRIPAEYADWGQGTQVTVSS

SEQ ID NO: 6 - protein, 121 aa, synthetic construct
  REGION 1..121: note = CD38 5F21 VHO1
EVQLQESGGGSVQAGGSLTLSCTASGLLFRLASMGWYRQAPGKERELIATITVGGKTNYKDSVQGRFIITRDNTGDNTKSTVTLQMNRLKPEDTAVYYCNTASPAVGADTWGQGTRVTVSS

SEQ ID NO: 7 - protein, 107 aa, synthetic construct
  REGION 1..107: note = CD38 LC2
DIQMTQSPSSLSASVGDRVTITCKASQDVSTVVAWYQQKPGKAPKRLIYSASYRYIGVPSRFSGSGSGTDFTFTISSLQPEDIATYYCQQHYSPPYTFGQGTKLEIK

SEQ ID NO: 8 - protein, 107 aa, synthetic construct
  REGION 1..107: note = CD38 LC3
DVVMTQSPLSLPVTLGQPASISCKASQDVSTVVAWYQQRPGQSPRRLIYSASYRYIGVPDRFSGSGSGTDFTLKISRVEAEDVGVYYCQQHYSPPYTFGQGTKLEIK

SEQ ID NO: 9 - protein, 107 aa, synthetic construct
  REGION 1..107: note = CD38 LC4
DIVMTQSHLSMSTSLGDPVSITCKASQDVHTEVAWYQQKPGQSPRRLIYRASYRYPGVPDRFTGSGAGTDFTFTISSVQAEDLAVYYCQQHYLPPYTFGGGTKLEIK

SEQ ID NO: 10 - protein, 107 aa, synthetic construct
  REGION 1..107: note = CD38 LC5
DIVMTQSHLSMSTSLGDPVSITCKASQDVFTNVAWYQQKPGQSPRRLIYFASYRYFGVPDRFTGSGAGTDFTFTISSVQAEDLAVYYCQQHYEPPYTFGGGTKLEIK

SEQ ID NO: 11 - protein, 107 aa, synthetic construct
  REGION 1..107: note = CD38 LC6
DIVMTQSHLSMSTSLGDPVSITCKASQDVYTVVAWYQQKPGQSPRRLIYSASYRYIGVPDRFTGSGAGTDFTFTISSVQAEDLAVYYCQQHYLPPYTFGGGTKLEIK

SEQ ID NO: 12 - protein, 120 aa, synthetic construct
  REGION 1..120: note = CD38 HC2
QVQLVQSGAEVKKPGSSVKVSCKASGYTFTDYWMQWVRQAPGQGLEWIGTIYPGDGDTGYAQKFQGRVTITADESTSTAYMELSSLRSEDTAVYYCARGDYYGSNSLDYWGQGTTVTVSS

SEQ ID NO: 13 - protein, 120 aa, synthetic construct
  REGION 1..120: note = CD38 HC3
QVQLVQSGAEVKKPGASVKVSCKASGYTFTDYWMQWVRQAPGQGLEWIGTIYPGDGDTGYAQKFQGRATLTADTSTSTVYMELSSLRSEDTAVYYCARGDYYGSNSLDYWGQGTTVTVSS

SEQ ID NO: 14 - protein, 120 aa, synthetic construct
  REGION 1..120: note = CD38 HC4
QVQLVQSGAEVAKPGTSVKLSCKASGYHFRDYWMQWVKQRPGQGLEWIGTIYPGYGDTGYAQKFQGKATLTADKSSKTVYMHLSSLASEDSAVYYCARGDYYGTNSLDYWGQGTSVTVSS

SEQ ID NO: 15 - protein, 120 aa, synthetic construct
  REGION 1..120: note = CD38 HC5
QVQLVQSGAEVAKPGTSVKLSCKASGYRFTDYWMQWVKQRPGQGLEWIGTIYPGDGDTGYAQKFQGKATLTADKSSKTVYMHLSSLASEDSAVYYCARGDYYGQNSLDYWGQGTSVTVSS

SEQ ID NO: 16 - protein, 120 aa, synthetic construct
  REGION 1..120: note = CD38 HC6
QVQLVQSGAEVAKPGTSVKLSCKASGYTFTDYWMQWVKQRPGQGLEWIGTIYPGDGDTGYAQKFQGKATLTADKSSKTVYMHLSSLASEDSAVYYCARGDYYGENSLDYWGQGTSVTVSS

SEQ ID NO: 17 - protein, 128 aa, synthetic construct
  REGION 1..128: note = CD38 5FIO VHO2
QVQLVQSGAEVKKPGASVKVSCKASGSRFDNYAMGWFRQAPGQEREFMGAISWSSGTTRYAQKVQGRVTMTRDTSTSTVYMELSSLRSEDTAVYYCAARYQPRYYDSGDMDGYEYDNWGQGTLVTVSS

SEQ ID NO: 18 - protein, 128 aa, synthetic construct
  REGION 1..128: note = CD38 5FIO VHO3
QVTLKESGPVLVKPTETLTLTCTGSGSRFDNYAMGWFRQPPGKEREFLAAISWSSGTTRYSTSLKSRLTISRDTSKSQVVLTMTNMDPVDTATYYCAARYQPRYYDSGDMDGYEYDNWGQGTLVTVSS

SEQ ID NO: 19 - protein, 128 aa, synthetic construct
  REGION 1..128: note = CD38 5FIO VHO4
EVQLVESGGGLVQPGGSLRLSCAASGSRFDNYAMGWFRQAPGKEREFVAAISWSSGTTRYVDSVKGRFTISRDNAKNSVYLQMNSLRAEDTAVYYCAARYQPRYYDSGDMDGYEYDNWGQGTLVTVSS

SEQ ID NO: 20 - protein, 128 aa, synthetic construct
  REGION 1..128: note = CD38 5FIO VHO5
DVQLQESGGGLVQAGHSLRLSCVGSGDRFDNYAMGWFRQAPGKEREFVAAISWSSGTTRYLDTVKGRFTISRDNAKSTVYLQMNSLKPEDTAVYYCAARYQNRFYDSGDMDGYEYDNWGQGTQVTVSS

SEQ ID NO: 21 - protein, 124 aa, synthetic construct
  REGION 1..124: note = CD38 5FIOK VHO2
QMQLVQSGAEVKKTGSSVKVSCKGSGRTFRNYPMAWFRQAPGQEREFMGGITWVGASTLYAQKAQDRVTITRDRSMSTAYMELSSLRSEDTAMYSCAAGRGIVAGRIPAEYADWGQGTLVTVSS

SEQ ID NO: 22 - protein, 124 aa, synthetic construct
  REGION 1..124: note = CD38 5FIOK VHO3
QVTLKESGPVLVKPTETLTLTCTGSGRTFRNYPMAWFRQPPGKEREFLAGITWVGASTLYSTSAKSRLTISRDTSKSQVVLTMTNMDPVDTATYSCAAGRGIVAGRIPAEYADWGQGTLVTVSS

SEQ ID NO: 23 - protein, 124 aa, synthetic construct
  REGION 1..124: note = CD38 5FIOK VHO4
QVQLVESGGGVVQPGRSLRLSCAGSGRTFRNYPMAWFRQAPGKEREFVAGITWVGASTLYADSAKGRFTISRDNSKNTVYLQMNSLRAEDTAVYSCAAGRGIVAGRIPAEYADWGQGTLVTVSS

SEQ ID NO: 24 - protein, 124 aa, synthetic construct
  REGION 1..124: note = CD38 5FIOK VHO5
DVQLQESGGGLVQAGGSLRLSCTGSGRTFYLYPMAWFRQAPGKEREFVAGITWVGASTNYADFAKGRFTISRDNAKNTVYLQMNSLKPEDTAVYSCAAGRGITAGRIPAEYADWGQGTQVTVSS

SEQ ID NO: 25 - protein, 124 aa, synthetic construct
  REGION 1..124: note = CD38 5FIOK VHO6
DVQLQESGGGLVQAGGSLRLSCTGSGRTFRNYPMAWFRQAPGKEREFVAGITWVGASTLYADFAKGRFTISRDNAKNTVYLQMNSLKPEDTAVYSCAAGRGINAGRIPAEYADWGQGTQVTVSS

SEQ ID NO: 26 - protein, 117 aa, synthetic construct
  REGION 1..117: note = CD38 5F21 VHO2
QMQLVQSGPEVKKPGTSVKVSCKASGLLFRLASMGWYRQARGQERELIGTITVGGKTNYAQKVQERVTITRDMSTSTAYMELSSLRSEDTAVYYCNTASPAVGADTWGQGTLVTVSS

SEQ ID NO: 27 - protein, 117 aa, synthetic construct
  REGION 1..117: note = CD38 5F21 VHO3
QVTLKESGPVLVKPTETLTLTCTASGLLFRLASMGWYRQPPGKERELIATITVGGKTNYSTSLKSRLTISRDTSKSQVVLTMTNMDPVDTATYYCNTASPAVGADTWGQGTLVTVSS

SEQ ID NO: 28 - protein, 117 aa, synthetic construct
  REGION 1..117: note = CD38 5F21 VHO4
EVQLVESGGGLVKPGGSLRLSCAASGLLFRLASMGWYRQAPGKERELVGTITVGGKTNYAAPVKGRFTISRDDSKNTVYLQMNSLKTEDTAVYYCNTASPAVGADTWGQGTLVTVSS

SEQ ID NO: 29 - protein, 117 aa, synthetic construct
  REGION 1..117: note = CD38 5F21 VHO5
DVQLQESGGGSVQAGGSLTLSCTASGWLFRLASMGWYRQAPGKEREMIAVITTGGRTNYKDSVQGRFIITRDNTKSTVTLQMNRLKPEDTAVYYCNTSMPVVGADEWGQGTRVTVSS

SEQ ID NO: 30 - protein, 117 aa, synthetic construct
  REGION 1..117: note = CD38 5F21 VHO6
DVQLQESGGGSVQAGGSLTLSCTASGLLFRLASMGWYRQAPGKERELIAIITKGGKTNYKDSVQGRFIITRDNTKSTVTLQMNRLKPEDTAVYYCNTTSPAVGADWWGQGTRVTVSS

SEQ ID NO: 31 - protein, 121 aa, synthetic construct
  REGION 1..121: note = Cris-7 - VH0
QVQLQQSGAELARPGASVKMSCKASGYTFTRSTMHWVKQRPGQGLEWIGYINPSSAYTNYNQKFKDKATLTADKSSSTAYMQLSSLTSEDSAVYYCASPQVHYDYNGFPYWGQGTLVTVSA

SEQ ID NO: 32 - protein, 121 aa, synthetic construct
  REGION 1..121: note = Cris-7 - VH1
QVQLVQSGAEVKKPGSSVKVSCKASGYTFTRSTMHWVRQAPGQGLEWIGYINPSSAYTNYAQKFQGRATLTADESTSTAYMELSSLRSEDTAVYYCASPQVHYDYNGFPYWGQGTLVTVSS

SEQ ID NO: 33 - protein, 121 aa, synthetic construct
  REGION 1..121: note = Cris-7 - VH2
EVQLVESGGGLVQPGRSLRLSCTASGYTFTRSTMHWVRQAPGKGLEWVGYINPSSAYTNYAASFKGRATLSADGSKSIAYLQMNSLKTEDTAVYYCASPQVHYDYNGFPYWGQGTLVTVSS

SEQ ID NO: 34 - protein, 121 aa, synthetic construct
  REGION 1..121: note = Cris-7 - VH3
QVQLVQSGSELKKPGASVKMSCKASGYTFTRSTMHWVRQAPGQGLEWMGYINPSSAYTNYAQGFTGRAVLSADTSVSTAYLQLCSLKAEDTAVYYCASPQVHYDYNGFPYWGQGTLVTVSS

SEQ ID NO: 35 - protein, 106 aa, synthetic construct
  REGION 1..106: note = cris-7 - VL0
QVVLTQSPAIMSAFPGEKVTMTCSASSSVSYMNWYQQKSGTSPKRWIYDSSKLASGVPARFSGSGSGTSYSLTISSMETEDAATYYCQQWSRNPPTFGGGTKLQIT

SEQ ID NO: 36 - protein, 106 aa, synthetic construct
  REGION 1..106: note = cris-7 - VL1
DIQMTQSPSSLSASVGDRVTITCRASSSVSYMNWYQQKPGKAPKRLIYDSSKLASGVPSRFSGSGSGTEFTLTISSLQPEDFATYYCQQWSRNPPTFGQGTKLEIK

SEQ ID NO: 37 - protein, 106 aa, synthetic construct
  REGION 1..106: note = cris-7 - VL2
AIRLTQSPFSLSASVGDRVTITCSASSSVSYMNWYQQKPAKAPKRWIYDSSKLASGVPSRFSGSGSGTDYTLTISSLQPEDAATYYCQQWSRNPPTFGQGTKLEIK

SEQ ID NO: 38 - protein, 109 aa, synthetic construct
  REGION 1..109: note = SP34 v8 LC
QTVVTQEPSLTVSPGGTVTLTCRSSTGAVTTSNYANWVQQKPGQAPRGLIGGTNKRAPGTPARFSGSLLGGKAALTLSGVQPEDEAEYYCALWYSNLWVFGGGTKLTVL

SEQ ID NO: 39 - protein, 125 aa, synthetic construct
  REGION 1..125: note = SP34 v8 HC
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVAWFAYWGQGTLVTVSS

SEQ ID NO: 40 - protein, 112 aa, synthetic construct
  REGION 1..112: note = 40G5 LC
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 41 - protein, 119 aa, synthetic construct
  REGION 1..119: note = 40G5 HC
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 42 - protein, 380 aa, synthetic construct
  REGION 1..380: note = K VHO1
MAWVWTLLFLMAAAQSIQAQVQLQESGGGLVQAGGSLRLSCAVSGNTISRYATGWFRQTPGNEREFVAAIRWTNGNTYYADSVEGRFTISRDSGKNTVYLQMNNLQPEDTAVYYCASRFLPYASSNAYHEALYNYDYWGQGTQVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 43 - protein, 380 aa, synthetic construct
  REGION 1..380: note = K VHO2
MAWVWTLLFLMAAAQSIQAQVQLQESGGGLVQAGGSLRLSCAVSGNTISRYATGWFRQTPGNEREFVAAIRWTNGNTYYADSVEGRFTISRDSGKNTVYLQMNNLQPEDTAVYYCASRFLPYASSNAYHESLYNYDYWGQGTQVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 44 - protein, 380 aa, synthetic construct
  REGION 1..380: note = K VHO3
MAWVWTLLFLMAAAQSIQAQVQLQESGGGLVQAGGSLRLSCAVSGNTISRYATGWFRQTPGNEREFVAAIRWENGNTYYADSVEGRFTISRDSGKNTVYLQMNNLQPEDTAVYYCASRFLPYASSNAYHEALYNYDYWGQGTQVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 45 - protein, 380 aa, synthetic construct
  REGION 1..380: note = K VHO4
MAWVWTLLFLMAAAQSIQAQVQLQESGGGLVQAGGSLRLSCAVSGNTISRYATGWFRQTPGNEREFVAAIRWENGNTYYADSVEGRFTISRDSGKNTVYLQMNNLQPEDTAVYYCASRFLPYASSNAYHESLYNYDYWGQGTQVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 46 - protein, 345 aa, synthetic construct
  REGION 1..345: note = CH1 VHO1
QVQLQESGGGLVQAGGSLRLSCAVSGNTISRYATGWFRQTPGNEREFVAAIRWTNGNTYYADSVEGRFTISRDSGKNTVYLQRFLPYASSNAYHEALYNYDYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 47 - protein, 345 aa, synthetic construct
  REGION 1..345: note = CH1 VHO2
QVQLQESGGGLVQAGGSLRLSCAVSGNTISRYATGWFRQTPGNEREFVAAIRWENGNTYYADSVEGRFTISRDSGKNTVYLQRFLPYASSNAYHESLYNYDYWGQGTQVTVSSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFLLYSKLTVDKSRWQQGNVFSCSVMHEALHNHYTQKSLSLSPGK

SEQ ID NO: 48 - protein, 361 aa, synthetic construct
  REGION 1..361: note = CH1 VHO3
QVQLQESGGGLVQAGGSLRLSCAVSGNTLSRYAMGWFRQAPGNEREFVAAIRWNNGNTHYADSVKGRFTISRDSAKNTVYLQMNNLQPEDTAVYYCASRFLPYASSNAYHEALYNYDYWGQGTQVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 49 - protein, 361 aa, synthetic construct
  REGION 1..361: note = CH1 VHO4
EVQLVESGGGLVQPGGSLRLSCAASGNTLSRYAMGWFRQAPGKEREFVAAIRWNNGNTHYVDSVKGRFTISRDNAKNSVYLQMNSLRAEDTAVYYCASRFLPYASSNAYHEALYNYDYWGQGTLVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 50 - protein, 361 aa, synthetic construct
  REGION 1..361: note = CH1 VHO5
QVTLKESGPVLVKPTETLTLTCTVSGNTLSRYAMGWFRQAPGKEREFVAAIRWNNGNTHYSTSLKSRLTISKDTSKSQVVLTMTNMDPVDTATYYCASRFLPYASSNAYHEALYNYDYWGQGTLVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 51 - protein, 361 aa, synthetic construct
  REGION 1..361: note = CH1 VHO6
QVQLVQSGAEVKKPGASVKVSCKASGNTLSRYAMGWFRQAPGQEREFVAAIRWNNGNTHYAQKFQGRVTMTRDTSTSTVYMELSSLRSEDTAVYYCASRFLPYASSNAYHEALYNYDYWGQGTLVTVSSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSRLTVDKSRWQQGNVFSCSVMHEALHNHYTQKSLSLSPGK

SEQ ID NO: 52 - protein, 9 aa, synthetic construct
  REGION 1..9: note = Sp34 epitope
QDGNEEMGG

SEQ ID NO: 53 - protein, 6 aa, synthetic construct
  REGION 1..6: note = Substrate sequence for MMP2 and MMP9
GPLGVR

SEQ ID NO: 54 - protein, 6 aa, synthetic construct
  REGION 1..6: note = Substrate sequence for MMP2 and MMP9
PLGLAR

SEQ ID NO: 55 - protein, 6 aa, synthetic construct
  REGION 1..6: note = Substrate sequence for MMP2 and MMP9
PLGLAG

SEQ ID NO: 56 - protein, 8 aa, synthetic construct
  REGION 1..8: note = Substrate sequence for MMP2 and MMP9
IPVSLRSG

SEQ ID NO: 57 - protein, 8 aa, synthetic construct
  REGION 1..8: note = Substrate sequence for MMP2 and MMP9
GPLGMLSQ

SEQ ID NO: 58 - protein, 10 aa, synthetic construct
  REGION 1..10: note = Substrate sequence for MMP3
RPKPVEVWRK

SEQ ID NO: 59 - protein, 8 aa, synthetic construct
  REGION 1..8: note = Substrate sequence for uPA
LSGRSDNH

SEQ ID NO: 60 - protein, 8 aa, synthetic construct
  REGION 1..8: note = Substrate sequence for uPA
TGRGPSWV

SEQ ID NO: 61 - protein, 11 aa, synthetic construct
  REGION 1..11: note = CD38 LC1 CDR1
KASQDVSTVVA

SEQ ID NO: 62 - protein, 7 aa, synthetic construct
  REGION 1..7: note = CD38 LC1 CDR2
SASYRYI

SEQ ID NO: 63 - protein, 9 aa, synthetic construct
  REGION 1..9: note = CD38 LC1 CDR3
QQHYSPPYT

SEQ ID NO: 64 - protein, 5 aa, synthetic construct
  REGION 1..5: note = CD38 HC1 CDR1
DYWMQ

SEQ ID NO: 65 - protein, 17 aa, synthetic construct
  REGION 1..17: note = CD38 HC1 CDR2
TIYPGDGDTGYAQKFQG

SEQ ID NO: 66 - protein, 11 aa, synthetic construct
  REGION 1..11: note = CD38 HC1 CDR3
GDYYGSNSLDY

SEQ ID NO: 67 - protein, 5 aa, synthetic construct
  REGION 1..5: note = CD38 5FIO VHO1 CDR1
NYAMG

SEQ ID NO: 68 - protein, 17 aa, synthetic construct
  REGION 1..17: note = CD38 5FIO VHO1 CDR2
AISWSSGTTRYLDTVKG

SEQ ID NO: 69 - protein, 19 aa, synthetic construct
  REGION 1..19: note = CD38 5FIO VHO1 CDR3
RYQPRYYDSGDMDGYEYDN

SEQ ID NO: 70 - protein, 5 aa, synthetic construct
  REGION 1..5: note = CD38 5F1K VHO1 CDR1
NYPMA

SEQ ID NO: 71 - protein, 17 aa, synthetic construct
  REGION 1..17: note = CD38 5F1K VHO1 CDR2
GITWVGASTLYADFAKG

SEQ ID NO: 72 - protein, 15 aa, synthetic construct
  REGION 1..15: note = CD38 5F1K VHO1 CDR3
GRGIVAGRIPAEYAD

SEQ ID NO: 73 - protein, 5 aa, synthetic construct
  REGION 1..5: note = CD38 5F21 VHO1 CDR1
LASMG

SEQ ID NO: 74 - protein, 16 aa, synthetic construct
  REGION 1..16: note = CD38 5F21 VHO1 CDR2
TITVGGKTNYKDSVQG

SEQ ID NO: 75 - protein, 9 aa, synthetic construct
  REGION 1..9: note = CD38 5F21 VHO1 CDR3
ASPAVGADT

SEQ ID NO: 76 - protein, 11 aa, synthetic construct
  REGION 1..11: note = CD38 LC4 CDR1
KASQDVHTEVA

SEQ ID NO: 77 - protein, 7 aa, synthetic construct
  REGION 1..7: note = CD38 LC4 CDR2
RASYRYP

SEQ ID NO: 78 - protein, 9 aa, synthetic construct
  REGION 1..9: note = CD38 LC4 CDR3
QQHYLPPYT

SEQ ID NO: 79 - protein, 11 aa, synthetic construct
  REGION 1..11: note = CD38 LC5 CDR1
KASQDVFTNVA

SEQ ID NO: 80 - protein, 7 aa, synthetic construct
  REGION 1..7: note = CD38 LC5 CDR2
FASYRYF

SEQ ID NO: 81 - protein, 9 aa, synthetic construct
  REGION 1..9: note = CD38 LC5 CDR3
QQHYEPPYT

SEQ ID NO: 82 - protein, 11 aa, synthetic construct
  REGION 1..11: note = CD38 LC6 CDR1
KASQDVYTVVA

SEQ ID NO: 83 - protein, 17 aa, synthetic construct
  REGION 1..17: note = CD38 HC4 CDR2
TIYPGYGDTGYAQKFQG

SEQ ID NO: 84 - protein, 11 aa, synthetic construct
  REGION 1..11: note = CD38 HC4 CDR3
GDYYGTNSLDY

SEQ ID NO: 85 - protein, 11 aa, synthetic construct
  REGION 1..11: note = CD38 HC5 CDR3
GDYYGQNSLDY

SEQ ID NO: 86 - protein, 11 aa, synthetic construct
  REGION 1..11: note = CD38 HC6 CDR3
GDYYGENSLDY

SEQ ID NO: 87 - protein, 16 aa, synthetic construct
  REGION 1..16: note = CD38 5FIO VHO2 CDR2
AISWSSGTTRYAQKVQ

SEQ ID NO: 88 - protein, 17 aa, synthetic construct
  REGION 1..17: note = CD38 5FIO VHO3 CDR2
AISWSSGTTRYSTSLKS

SEQ ID NO: 89 - protein, 17 aa, synthetic construct
  REGION 1..17: note = CD38 5FIO VHO3 CDR2
AISWSSGTTRYVDSVKG

SEQ ID NO: 90 - protein, 19 aa, synthetic construct
  REGION 1..19: note = CD38 5FIO VHO4 CDR3
RYQNRFYDSGDMDGYEYDN

SEQ ID NO: 91 - protein, 17 aa, synthetic construct
  REGION 1..17: note = CD38 5FIOK VHO2 CDR2
GITWVGASTLYAQKAQD

SEQ ID NO: 92 - protein, 17 aa, synthetic construct
  REGION 1..17: note = CD38 5FIOK VHO3 CDR2
GITWVGASTLYSTSAKS

SEQ ID NO: 93 - protein, 17 aa, synthetic construct
  REGION 1..17: note = CD38 5FIOK VHO4 CDR2
GITWVGASTLYADSAKG

SEQ ID NO: 94 - protein, 5 aa, synthetic construct
  REGION 1..5: note = CD38 5FIOK VHO5 CDR1
LYPMA

SEQ ID NO: 95 - protein, 17 aa, synthetic construct
  REGION 1..17: note = CD38 5FIOK VHO5 CDR2
GITWVGASTNYADFAKG

SEQ ID NO: 96 - protein, 15 aa, synthetic construct
  REGION 1..15: note = CD38 5FIOK VHO5 CDR3
GRGITAGRIPAEYAD

SEQ ID NO: 97 - protein, 15 aa, synthetic construct
  REGION 1..15: note = CD38 5FIOK VHO6 CDR3
GRGINAGRIPAEYAD

SEQ ID NO: 98 - protein, 16 aa, synthetic construct
  REGION 1..16: note = CD38 5F21 VHO2 CDR2
TITVGGKTNYAQKVQE

SEQ ID NO: 99 - protein, 16 aa, synthetic construct
  REGION 1..16: note = CD38 5F21 VHO3 CDR2
TITVGGKTNYSTSLKS

SEQ ID NO: 100 - protein, 16 aa, synthetic construct
  REGION 1..16: note = CD38 5F21 VHO4 CDR2
TITVGGKTNYAAPVKG

SEQ ID NO: 101 - protein, 16 aa, synthetic construct
  REGION 1..16: note = CD38 5F21 VHO5 CDR2
VITTGGRTNYKDSVQG

SEQ ID NO: 102 - protein, 9 aa, synthetic construct
  REGION 1..9: note = CD38 5F21 VHO5 CDR3
SMPVVGADE

SEQ ID NO: 103 - protein, 16 aa, synthetic construct
  REGION 1..16: note = CD38 5F21 VHO6 CDR2
IITKGGKTNYKDSVQG

SEQ ID NO: 104 - protein, 9 aa, synthetic construct
  REGION 1..9: note = CD38 5F21 VHO6 CDR3
TSPAVGADW

SEQ ID NO: 105 - protein, 5 aa, synthetic construct
  REGION 1..5: note = Cris-7 - VH0 CDR1
RSTMH

SEQ ID NO: 106 - protein, 17 aa, synthetic construct
  REGION 1..17: note = Cris-7 - VH0 CDR2
YINPSSAYTNYNQKFKD

SEQ ID NO: 107 - protein, 12 aa, synthetic construct
  REGION 1..12: note = Cris-7 - VH0 CDR3
PQVHYDYNGFPY

SEQ ID NO: 108 - protein, 17 aa, synthetic construct
  REGION 1..17: note = Cris-7 - VH1 CDR2
YINPSSAYTNYAQKFQG

SEQ ID NO: 109 - protein, 17 aa, synthetic construct
  REGION 1..17: note = Cris-7 - VH2 CDR2
YINPSSAYTNYAASFKG

SEQ ID NO: 110 - protein, 17 aa, synthetic construct
  REGION 1..17: note = Cris-7 - VH3 CDR2
YINPSSAYTNYAQGFTG

SEQ ID NO: 111 - protein, 10 aa, synthetic construct
  REGION 1..10: note = cris-7 - VL0 CDR1
SASSSVSYMN

SEQ ID NO: 112 - protein, 7 aa, synthetic construct
  REGION 1..7: note = cris-7 - VL0 CDR2
DSSKLAS

SEQ ID NO: 113 - protein, 9 aa, synthetic construct
  REGION 1..9: note = cris-7 - VL0 CDR3
QQWSRNPPT

SEQ ID NO: 114 - protein, 10 aa, synthetic construct
  REGION 1..10: note = cris-7 - VL1 CDR1
RASSSVSYMN

SEQ ID NO: 115 - protein, 14 aa, synthetic construct
  REGION 1..14: note = SP34 v8 LC CDR1
RSSTGAVTTSNYAN

SEQ ID NO: 116 - protein, 7 aa, synthetic construct
  REGION 1..7: note = SP34 v8 LC CDR2
GTNKRAP

SEQ ID NO: 117 - protein, 9 aa, synthetic construct
  REGION 1..9: note = SP34 v8 LC CDR3
ALWYSNLWV

SEQ ID NO: 118 - protein, 10 aa, synthetic construct
  REGION 1..10: note = SP34 v8 HC CDR1
GFTFNTYAMN

SEQ ID NO: 119 - protein, 12 aa, synthetic construct
  REGION 1..12: note = SP34 v8 HC CDR2
RIRSKYNNYATY

SEQ ID NO: 120 - protein, 14 aa, synthetic construct
  REGION 1..14: note = SP34 v8 HC CDR3
HGNFGNSYVAWFAY

SEQ ID NO: 121 - protein, 17 aa, synthetic construct
  REGION 1..17: note = 40G5 LC CDR1
KSSQSLLNSRTRKNYLA

SEQ ID NO: 122 - protein, 7 aa, synthetic construct
  REGION 1..7: note = 40G5 LC CDR2
WASTRES

SEQ ID NO: 123 - protein, 8 aa, synthetic construct
  REGION 1..8: note = 40G5 LC CDR3
TQSFILRT

SEQ ID NO: 124 - protein, 5 aa, synthetic construct
  REGION 1..5: note = 40G5 HC CDR1
NYYIH

SEQ ID NO: 125 - protein, 17 aa, synthetic construct
  REGION 1..17: note = 40G5 HC CDR2
WIYPGDGNTKYNEKFKG

SEQ ID NO: 126 - protein, 10 aa, synthetic construct
  REGION 1..10: note = 40G5 HC CDR3
DSYSNYYFDY

SEQ ID NO: 127 - protein, 125 aa, synthetic construct
  REGION 1..125: note = SP34 heavy chain variable domain sequence
EVQLVESGGGLVQPGGSLKLSCAASGFTFNTYAMNWVRQAPGKGLEWVARIRSKYNNYATYYADSVKDRFTISRDDSKNTAYLQMNNLKTEDTAVYYCVRHGNFGNSYVSWFAYWGQGTLVTVSS

SEQ ID NO: 128 - protein, 4 aa, synthetic construct
GGSG

SEQ ID NO: 129 - protein, 5 aa, synthetic construct
GGSGG

SEQ ID NO: 130 - protein, 5 aa, synthetic construct
GGGGS

SEQ ID NO: 131 - protein, 5 aa, synthetic construct
GSGSG

SEQ ID NO: 132 - protein, 5 aa, synthetic construct
GSGGG

SEQ ID NO: 133 - protein, 5 aa, synthetic construct
GGGSG

SEQ ID NO: 134 - protein, 5 aa, synthetic construct
GSSSG

SEQ ID NO: 135 - protein, 5 aa, synthetic construct
  VARIANT 1..5: note = The entire sequence of amino acids 1-5 can be repeated one or more times
GSGGS

SEQ ID NO: 136 - protein, 4 aa, synthetic construct
  VARIANT 1..4: note = The entire sequence of amino acids 1-4 can be repeated one or more times.
GGGS

SEQ ID NO: 137 - protein, 4 aa, synthetic construct
  VARIANT 1..4: note = The entire sequence of amino acids 1-4 can be repeated one or more times.
GGGG

SEQ ID NO: 138 - protein, 4 aa, synthetic construct
  VARIANT 1..4: note = The entire sequence of amino acids 1-4 can be repeated one or more times.
GSGS

SEQ ID NO: 139 - protein, 5 aa, synthetic construct
  VARIANT 1..5: note = The entire sequence of amino acids 1-5 can be repeated one or more times.
EAAAK

SEQ ID NO: 140 - protein, 8 aa, synthetic construct
  VARIANT 1..8: note = The entire sequence of amino acids 1-8 can be repeated one or more times.
  VARIANT 1: note = X can be Ala, Lys, or Glu
  VARIANT 3: note = X can be Ala, Lys, or Glu
  VARIANT 5: note = X can be Ala, Lys, or Glu
  VARIANT 7: note = X can be Ala, Lys, or Glu
XPXPXPXP

SEQ ID NO: 141 - protein, 4 aa, synthetic construct
  VARIANT 1..4: note = The entire sequence of amino acids 1-4 can be repeated one or more times.
APAP